(19) United States
(12) Patent Application Publication
(10) Pub. No.: US 2016/0235781 A1
(43) Pub. Date: Aug. 18, 2016

(54) DIURETIC INDUCED ALTERATIONS OF PLASMA VOLUME

(71) Applicant: MAST THERAPEUTICS, INC., San Diego, CA (US)

(72) Inventor: R. Martin EMANUELE, San Diego, CA (US)

(73) Assignee: MAST THERAPEUTICS, INC., San Diego, CA (US)

(21) Appl. No.: 15/029,614

(22) PCT Filed: Oct. 16, 2014
(86) PCT No.: PCT/US2014/060982

§ 371 Date: Apr. 14, 2016

Related U.S. Application Data
(60) Provisional application No. 61/891,856, filed on Oct. 16, 2013.

(51) Int. Cl. A61K 31/77; A61K 9/00

(52) U.S. Cl. CPC A61K 31/77; A61K 9/0019

(57) ABSTRACT

Provided are uses of a poloxamer and methods of administering a poloxamer for treating hemo-concentration, such as hemo-concentration resulting from dehydration and/or diuresis in a subject. Administration of a poloxamer prevents, treats or otherwise reduces adverse effects of hemo-concentration, dehydration and/or diuresis.

### RELATED APPLICATIONS

Benefit of priority is claimed to U.S. Provisional Application Ser. No. 61/891,856, filed Oct. 16, 2013, entitled “TREATMENT OF DIURETIC INDUCED ALTERATIONS OF PLASMA VOLUME,” to Marty Emanuele (R. Martin Emanuele).

This application is related to U.S. provisional application Ser. No. 62/021,697 to R. Martin Emanuele and Mannarsamy Balasubramanian, filed Jul. 7, 2014, entitled “A POLOXAMER COMPOSITION FREE OF LONG CIRCULATING MATERIAL, METHODS FOR PRODUCTION THEREOF AND USES THEREOF.”

Where permitted, the subject matter of each of the above-referenced applications is incorporated by reference in its entirety.

### FIELD OF THE INVENTION

Provided are methods and uses of poloxamers for treating certain side effects and complications resulting from hemo-concentration, such as from diuresis and/or dehydration in human and animal subjects. In particular, the uses and treatments include those in which diuretic therapy results in hemo-concentration and microvascular hemodynamic alterations

### BACKGROUND

Diuretics are clinically important therapeutics for managing extracellular fluid volume overload. Their use has been linked to an increase in mortality, worsening kidney function, and progression of organ failure in certain clinical disorders. Studies indicate an association between high dose diuretics and mortality.

In patients with compromised kidney function and other disorders, water can accumulate in the body. Over time, this results in an expansion of the volume of fluid in circulation. The larger volume of circulating fluid effectively dilutes proteins and red blood cells (RBCs) in the blood resulting in dilutional anemia. The body often compensates for this and attempts to correct the dilutional amenia by increasing the levels of RBCs and plasma proteins. The increase in fluid volume can continue leading to development of congestion. Diuretics are administered to reduce the fluid volume, and thereby relieve the congestion. The relatively rapid loss of water from the circulation resulting from the diuretic therapy results in a relative increase in the concentration of blood cells, especially RBCs, and plasma proteins in the blood, a consequent impairment of the circulation, especially the microcirculation, and the potential for dehydration.

These processes and treatments, as well as others, lead to hemo-concentration of cells and proteins in the blood. There is a need for a treatments of this hemo-concentration and microvascular hemodynamic dysfunction due to diuresis and/or dehydration and other causes.

### SUMMARY

Provided herein are methods of treatments and compositions and uses thereof for treatment of hemo-concentration and microvascular hemodynamic dysfunction resulting from dehydration and diuresis and disease or conditions that result in hemo-concentration.

Provided are methods for treating or preventing a complication of hemo-concentration in a subject in need thereof. The hemo-concentration is treated by administering a poloxamer. The methods can be used to treat hemo-concentration or reduce the risk therefor in subjects at risk of developing hemo-concentration. Hemo-concentration can occur from variety of conditions and/or treatments, including diuresis and dehydration.

In particular, the methods include identifying a subject who exhibits hemo-concentration, a complication of hemo-concentration, or who is at risk of hemo-concentration, and administering a polyoxyethylene/polyoxypropylene copolymer (poloxamer) to achieve a circulating concentration sufficient to treat or prevent the complication.

The copolymers include those having the following formula:

HO(C₂H₄O)_{a′}—(C₃H₆O)_{b}—(C₂H₄O)ₐH,

where a′ and a are the same or different and each is an integer such that the hydrophile portion represented by (C₂H₄O) constitutes at or about 60% to 90% by weight of the compound, and b is an integer such that the hydrophobe represented by (C₃H₆O) has a molecular weight of approximately or is 1300 to 2300 Daltons (Da), such as 1750 Da, and the total molecular weight of the copolymer is approximately or is 8400 to 8800 Da. In certain embodiments, the polyoxypropylene hydrophobe has a molecular weight of at or about 1800 Da and the hydrophilic polyoxyethylene content is about 80% of the total molecular weight. In some embodiments, a′ and a are the same or different, where each is an integer from 5 to 150, inclusive, and b is an integer from 15 to 75, inclusive, such as where a′ and a are from 70 to 105, inclusive, and b is from 15 to 75, inclusive. In some embodiments, the copolymer has reduced impurities so that the polydispersity value is less than or equal to 1.07.

Provided are the methods where the polyoxyethylene/polyoxypropylene copolymer is one where a′ and a are the same or about the same and are about or are 78, 79 or 80, and b is about or is 27, 28, 29 or 30, such as where a′ and a are 80, and b is 27. In some embodiments, the P188 copolymer is a poloxamer with a hydrophobe portion having a molecular weight of about or is 1400 to 2000 Da, such as a molecular weight of 1500 to 2100 Da or 1700 to 1900 Da, such as where the molecular weight of the hydrophobe (C₃H₆O) is about or is 1750 Da. In some embodiments, the hydrophile portion constitutes approximately 70% to 90% or 70% to 90% by weight of the copolymer.

In some embodiments, the copolymer is purified to reduce low molecular weight substances. In others, the copolymer is poloxamer 188.

Included are methods where the poloxamer has the following chemical formula:

HO(C₂H₄O)_{a′}—(C₃H₆O)_{b}—(C₂H₄O)ₐH, where

the hydrophobe portion represented by (C₃H₆O) has a molecular weight of approximately or at 1700 to 1800 Da, and a total molecular weight between 8400 and 8800 Da. In others, b is 27, the hydrophile portion represented by (C₂H₄O) constitutes 80% to 81% of the total molecular weight of the poloxamer, and the total molecular weight is 1750 Da. In yet others, a′ and a, which can be the same or different, are integers from 5 to 150, b is an integer from 15 to 75 or 15 to 72, the hydrophile portion represented by (C₂H₄O) constitutes 80% to 81% of the total molecular weight of the poloxamer, and the total molecular weight is 1800 Da.

In some embodiments, the hydrophobe portion represented by (C₃H₆O) has a molecular weight of approximately or at 1700 to 1800 Da, and the copolymer has a total molecular weight between 8400 to 8800 Da. In other embodiments, b is 27, the hydrophile constitutes 80% to 81% of the total molecular weight of the poloxamer, and the molecular weight is 1750 Da.

In other embodiments s, a′ and a, which can be the same or different, are integers between 5 and 150; b is an integer between 15 and 75 or 15 and 72, the hydrophile constitutes 80% to 81% of the total molecular weight of the poloxamer, and the molecular weight is 1800 Da. In some embodiments, the copolymer is a long-circulating material-free (LCMF) poloxamer.

In some embodiments, the copolymer is a long-circulating material-free (LCMF) poloxamer, such as a LCMF 188 that, when administered to a subject, does not contain a component that is or gives rise to in the plasma of the subject a material or component that has a circulating half-life (t_{1/2}) that is more than about 1.5-fold or 1.5-fold greater than the half-life of the main peak in the distribution of the copolymer preparation, or such that all components have a circulating half-life that is within 5-fold of the half-life of the main peak. In some embodiments, the LCMF poloxamer is a polyoxyethylene/polyoxypropylene copolymer that has the following formula:

HO(C₂H₄O)_{a′}—(C₃H₆O)_{b}—(C₂H₄O)ₐH,

where a′ and a are each integers, the same or different, such that the molecular weight of the hydrophobe portion (C₃H₆O) is between approximately 1300 and 2300 Da; b is an integer such that the percentage of the hydrophile portion (C₂H₄O) is between approximately 60% and 90% by weight of the total molecular weight of the copolymer; no more than 1.5% of the total components in the distribution of the copolymer are low molecular weight components having an average molecular weight of less than 4500 Da; no more than 1.5% of the total components in the distribution of the copolymer are high molecular weight components having an average molecular weight of greater than 13,000 Da; the polydispersity value of the copolymer is less than or approximately less than 1.07; and the half-life of any component of the distribution, when the copolymer is administered to a subject, is no more than 5.0-fold longer than the half-life of the main peak in the distribution of the copolymer.

Provided are methods including a polyoxyethylene/polyoxypropylene copolymer that is a poloxamer with a hydrophobe portion (C₃H₆O) having a molecular weight of about 1400 to 2000 Da or 1400 to 2000 Da, such as where the molecular weight of the hydrophobe is about or is 1750 Da, and a hydrophile portion (C₂H₄O) constituting approximately 70% to 90% or 70% to 90% by weight of the copolymer. The average molecular weight of the polyoxyethylene/polyoxypropylene copolymer is 8400 to 8800 Da. In some embodiments, the percentage of high molecular weight components in the preparation greater than 13,000 Da constitutes less than 1% of the total distribution of components of the poloxamer preparation, such as where the percentage of high molecular weight components in the preparation greater than 13,000 daltons constitutes less than 0.9%, 0.8%, 0.7%, 0.6%, 0.5% or less of the total distribution of components of the poloxamer preparation, and, when administered, does not result in a component that exhibits a circulating half-life that is greater than the circulating half-life of the main peak. In some embodiments, all components have a circulating t_{1/2} that is within 2, 3 or 4-fold that of the main peak.

In some embodiments, the poloxamer, such as the P188, that is administered is produced or further purified so that, not only are the low molecular weight components (LMW) removed, but also high molecular weight components. Such preparations are referred to a longer circulation material free (LCMF) poloxamer. The LCMF poloxamer, particularly an LCMF 188 poloxamer, is described in U.S. provisional application Ser. No. 62/021,697 (see, also International PCT application No. PCT/US14/45627) and herein.

In such preparation, all components in the distribution of the copolymer, when administered to a subject, exhibit a half-life in the plasma of the subject that is no more than 4.0-fold, 3.0-fold, 2.0 fold or 1.5-fold longer than the half-life of the main peak in the distribution of the copolymer, such as a half-life in the plasma of the subject that is no more than 1.5-fold longer than the half-life of the main peak in the distribution of the copolymer. In others embodiments, all of the components of the polymeric distribution clear from the circulation at approximately the same rate. When administered to a subject, any component(s) in the distribution of the copolymer exhibits a half-life in the plasma of the subject that is no more than the half-life of the main peak in the distribution of the copolymer. In some embodiments, all of the components in the distribution of the copolymer, when administered to a human subject, exhibit a half-life in the plasma of the subject that is no more than 30 hours, 25 hours, 20 hours, 15 hours, 10 hours, 9 hours, 8 hours or 7 hours, such as a half-life in the plasma of the subject that is no more than 10 hours.

In some embodiments, polyoxyethylene/polyoxypropylene copolymer administered is an LCMF poloxamer that has the following formula:

HO(C₂H₄O)_{a′}—(C₃H₆O)_{b}—(C₂H₄O)ₐH, where.

the LCMF poloxamer is a poloxamer 188 in which the percentage of high molecular weight components in the preparation with a molecular weight greater than 13,000 Da constitutes less than 1% of the total distribution of components of the poloxamer preparation, and, when administered, does not result in a component with a circulating half-life that is greater than the circulating half-life of the main peak. The total molecular weight of the polyoxyethylene/polyoxypropylene copolymer is approximately 8400 to 8800 Da.

Provided are methods where the polyoxyethylene/polyoxypropylene copolymer that is administered has reduced impurities so that the polydispersity value is less than or equal to 1.07. In some embodiments, the polydispersity value is less than 1.06, 1.05, 1.04, 1.03, or less.

The subject to be treated can be any animal, including humans and non-human animals, particularly pets and domestic animals. The subject who is treated can be one who exhibits hemo-concentration, a complication of hemo-concentration, or is at risk of hemo-concentration. In some embodiments, the hemo-concentration is the result of diuresis and/or dehydration. In some embodiments, the copolymer is administered to prevent or reduce the complications of, or the risk of developing, diuresis or dehydration. The subject can be one who has an underlying condition that has lead to the risk of developing or developing hemo-concentration.

In some embodiments, the polyoxyethylene/polyoxypropylene copolymer is administered to a subject prior to, concomitant with, or after the administration of another agent, such as an agent for treating an underlying condition. In some embodiments, the other agent is a diuretic.

The poloxamer can be administered with an agent for treatment of the underlying condition or intermittently the agent, or before or after administration of the agent. Exemplary of such agents are diuretics. The copolymer can be administered before, after, or with the diuretic. The diuretic can be a thiazide diuretic, loop diuretic, potassium-sparing diuretic, carbonic anhydrase inhibitor, osmotic diuretic, and combinations thereof.

In some embodiments, the method is for treating a complication of or side effect of diuresis or dehydration, including where the subject has been treated with a diuretic, such as where the subject is experiencing diuresis associated with diuretic treatment. In some embodiments, diuretic treatment is administered to ameliorate a condition such as a kidney related condition, high blood pressure, a liver condition, a heart-related condition and glaucoma. In some embodiments, diuresis results in a side-effect such as electrolyte imbalance, excessive diuresis, dehydration, arrhythmia, an alteration of plasma volume, increased hemo-concentration of at least one plasma protein, hemo-concentration of red blood cells, and combinations thereof. In some embodiments, the plasma protein is an acute phase reactant protein, such as fibrinogen.

In some embodiments, the copolymer is administered to a subject that has an underlying disease or condition, such as, but are not limited to, atherosclerosis, diabetes, heart failure, vasculitis, Raynaud's disease, sickle cell disease and polycythemia.

In some embodiments, the subject is a post-surgical patient. In others, the subject has acute heart failure. In others, the subject has dehydration, such as dehydration that results from strenuous exercise.

Provided are methods and uses where treatment with the polyoxyethylene/polyoxy-propylene copolymer results in a concentration of the polyoxyethylene/polyoxypropylene copolymer in the circulation of the subject of from 0.05 mg/mL to 10 mg/mL, such as from 0.2 mg/mL to 4.0 mg/mL, or about 0.5 mg/mL to 1.5 mg/mL or 0.5 mg/mL to 1.5 mg/mL. In some embodiments, the concentration of the polyoxyethylene/polyoxypropylene copolymer in the circulation is the peak concentration. In other embodiments, the concentration in circulation is the concentration in circulation at steady state. In some embodiments, the concentration of the polyoxyethylene/polyoxypropylene copolymer in the circulation is targeted for up to 72 hours following administration.

The poloxamer can be administered by any suitable route of administration. Generally, the copolymer is administered intravenously, such as by intravenous infusion. In others, the copolymer is administered by bolus injection. Treatment and dosage is selected to achieve a sufficient circulating concentration of poloxamer that effects treatment of a complication of the hemo-concentration. Generally the poloxamer is administered a plurality of times to achieve and maintain the circulating concentration. For example, the poloxamer can be administered a plurality of times for at least 12 hours up to 4 days, or at least 12 hours up to 3 days, or at least 1 day, days or 3 days. In some embodiments, when the copolymer, is administered a second time, he second treatment is sufficient to result in a concentration of the polyoxyethylene/polyoxypropylene copolymer in the circulation of the subject of from 0.05 mg/mL to 4.0 mg/mL, such as from about 0.2 mg/mL to about 2 mg/mL. The copolymer is administered as a single continuous IV infusion, a plurality of continuous IV infusions, a single IV bolus administration, or a plurality of IV bolus administrations, or a combination thereof. The subject is a human or veterinary (animal) subject. In some embodiments, the subject is a non-human mammal.

Also provided are compositions for use in treating or preventing complications of hemo-concentration of blood and compositions for use for formulating a medicament for treating or preventing complications of hemo-concentration of blood. The compositions for are those described above for the methods, are for use for treatment or prevention of conditions involving hemo-concentration, including diuresis and dehydration. The compositions for use contain a therapeutically effective amount of a polyoxyethylene/polyoxypropylene copolymer. As above for the methods, in some embodiments, the copolymer has the following formula:

HO(C₂H₄O)_{a′}—(C₃H₆O)_{b}—(C₂H₄O)ₐH,

where a′ and a are the same or different and each is an integer such that the hydrophile portion represented by (C₂H₄O) constitutes at or about 60% to 90% by weight of the compound, and b is an integer such that the hydrophobe represented by (C₃H₆O) has a molecular weight of approximately or 1300 to 2300 Da, such as approximately or 1750 Da, where the total molecular weight of the copolymer is approximately or is 8400 to 8800 Da, or the hydrophobe has a molecular weight of at or about 1800 Da and the hydrophilic polyoxyethylene content is about 80% of the total molecular weight. In some embodiments, a′ and a can be the same or different and each is an integer from 5 to 150, inclusive, and b is an integer from 15 to 75, inclusive. In others, a′ and a are each an integer from 70 to 105, inclusive, and b is an integer from 15 to 75, inclusive. In some embodiments, the polyoxyethylene/polyoxypropylene copolymer has reduced impurities, such as where the polydispersity value is less than or equal to 1.07.

In some embodiments of the uses, the polyoxyethylene/polyoxypropylene copolymer is poloxamer 188 (P188) which has the following formula:

HO(C₂H₄O)_{a′}—(C₃H₆O)_{b}—(C₂H₄O)ₐH,

where a′ and a are the same and are about 78, 79 or 80, and b is about 27, 28, 29 or 30, such as where a and a′ are 80 and b is 27. In some embodiments, the copolymer is purified to reduce low molecular weight substances.

In some embodiments of the uses, the polyoxyethylene/polyoxypropylene copolymer is a poloxamer with a hydrophobe represented by (C₃H₆O) having a molecular weight of about 1400 to 2000 Da or 1400 to 2000 Da, or 1500 to 2100 Da, or 1700 to 1900 Da, or is 1800 Da, and a hydrophile portion constituting approximately 70% to 90% or 70% to 90% by weight of the copolymer. In some embodiments, the copolymer is poloxamer 188.

In some embodiments of the uses, the composition or use contains a copolymer that is a longer circulating material-free (LCMF) poloxamer, such as a LCMF 188 as described above for the methods. As noted above, the LCMF poloxamer is manufactured or purified such that, when administered to a subject, it does not contain a component that is or gives rise to in the plasma of the subject, a material or component that has a circulating half-life (t_{1/2}) that is more than about 1.5-fold or 1.5-fold greater than the half-life of the main peak in the distribution of the copolymer preparation, or such that all components have a circulating half-life that is within 5-fold of the half-life of the main peak. In some embodiments, the LCMF poloxamer is a polyoxyethylene/polyoxypropylene copolymer that has the following formula:

HO(C₂H₄O)_{a′}—(C₃H₆O)_{b}—(C₂H₄O)ₐH,

where a′ or a are each integers such that the molecular weight of the hydrophobe portion (C₃H₆O) is between approximately 1300 and 2300 Da, and a′ and a are the same or different; b is an integer such that the percentage of the hydrophile portion (C₂H₄O) is between approximately 60% and 90% by weight of the total molecular weight of the copolymer; no more than 1.5% of the total components in the distribution of the copolymer are low molecular weight components having an average molecular weight of less than 4500 Da; no more than 1.5% of the total components in the distribution of the copolymer are high molecular weight components having an average molecular weight of greater than 13,000 Da; the polydispersity value of the copolymer is less than or approximately less than 1.07; and the half-life of any component the distribution, when the copolymer is administered to a subject, is no more than 5.0-fold longer than the half-life of the main peak in the distribution of the copolymer.

In some embodiments, all components in the distribution of the copolymer, when administered to a subject, exhibit a half-life in the plasma of the subject that is no more than 4.0-fold, 3.0-fold, 2.0 fold or 1.5-fold longer than the half-life of the main peak in the distribution of the copolymer, such as a half-life in the plasma of the subject that is no more than 1.5-fold longer than the half-life of the main peak in the distribution of the copolymer. In others embodiments, all of the components of the polymeric distribution clear from the circulation at approximately the same rate. When administered to a subject, any component(s) in the distribution of the copolymer exhibits a half-life in the plasma of the subject that is no more than the half-life of the main peak in the distribution of the copolymer. In some embodiments, all of the components in the distribution of the copolymer, when administered to a human subject, exhibit a half-life in the plasma of the subject that is no more than 30 hours, 25 hours, 20 hours, 15 hours, 10 hours, 9 hours, 8 hours or 7 hours, such as a half-life in the plasma of the subject that is no more than 10 hours.

Provided are compositions and uses in which the poloxamer is a polyoxyethylene/polyoxypropylene copolymer that is a poloxamer with a hydrophobe portion (C₃H₆O) having a molecular weight of about 1400 to 2000 Da or 1400 to 2000 Da, such as where the molecular weight of the hydrophobe is about or is 1750 Da, and a hydrophile portion (CH₂CH₂O) constituting approximately 70% to 90% or 70% to 90% by weight of the copolymer. In some embodiments, the average molecular weight of the polyoxyethylene/polyoxypropylene copolymer is 8400 to 8800 Da. In some embodiments, the percentage of high molecular weight components in the preparation greater than 13,000 Da constitutes less than 1% of the total distribution of components of the poloxamer preparation, such as where the percentage of high molecular weight components in the preparation greater than 13,000 daltons constitutes less than 0.9%, 0.8%, 0.7%, 0.6%, 0.5% or less of the total distribution of components of the poloxamer preparation, and, when administered, does not result in a component that exhibits a circulating half-life that is greater than the circulating half-life of the main peak. In some embodiments, all components have a circulating t_{1/2} that is within 2, 3 or 4-fold that of the main peak.

In some embodiments, where the LCMF poloxamer is a polyoxyethylene/polyoxypropylene copolymer with the following formula:

HO(C₂H₄O)_{a′}—(C₃H₆O)_{b}—(C₂H₄O)ₐH,

the LCMF poloxamer is a poloxamer 188 in which the percentage of high molecular weight components in the preparation with a molecular weight greater than 13,000 Da constitutes less than 1% of the total distribution of components of the poloxamer preparation, and, when administered, does not result in a component with a circulating half-life that is greater than the circulating half-life of the main peak. The total molecular weight of the polyoxyethylene/polyoxypropylene copolymer is approximately 8400 to 8800 Da.

Provided are compositions for use and uses where the polyoxyethylene/polyoxypropylene copolymer has reduced impurities, so that the polydispersity value is less than or equal to 1.07. In some embodiments, the polydispersity value is less than 1.06, 1.05, 1.04, 1.03 or less.

The conditions for which the compositions are used for treatment or formulated for treatment are any involving hemo-concentration or where there is a risk of complications from hemo-concentration. In some embodiments, the treatment is for hemo-concentration, a complication of hemo-concentration, or is at risk therefor, such as hemo-concentration resulting from diuresis and/or dehydration. In some embodiments, the hemo-concentration is the result of diuresis. In some embodiments, the copolymer used for treatment as prophylactic to prevent, treat, or reduce the complications of, or the risk of developing diuresis or dehydration. The subject, in some embodiments, is treated with a diuretic. The uses of the copolymer can include regimens in which the copolymer is administered a plurality of times as described above for the methods and/or is administered with, before or after, another agent for treating an underlying condition. For example, the regimen can be one in which the copolymer is administered before, after, or with the diuretic. The diuretic can be a thiazide diuretic, loop diuretic, potassium-sparing diuretic, carbonic anhydrase inhibitor, osmotic diuretic, and combinations thereof.

In some embodiments, the composition or use is for treating a complication of diuresis or dehydration, including where the subject has been treated with a diuretic, such as where the subject is experiencing diuresis associated with diuretic treatment. In some embodiments, diuretic treatment is administered to ameliorate a condition such as a kidney related condition, high blood pressure, a liver condition, a heart-related condition and glaucoma. In others, diuresis results in a side-effect such as electrolyte imbalance, excessive diuresis, dehydration, arrhythmia, an alteration of plasma volume, increased hemo-concentration of at least one plasma protein, hemo-concentration of red blood cells, and combinations thereof. In some embodiments, the plasma protein is an acute phase reactant protein, such as fibrinogen.

In some embodiments, the copolymer is for treatment of hemo-concentration in a subject who has an underlying disease or condition, such as, but are not limited to, atherosclerosis, diabetes, heart failure, vasculitis, Raynaud's disease, sickle cell disease and polycythemia. In some embodiments, the subject is a post-surgical patient, such as a subject with post-surgical hemo-concentration. In others, the subject has dehydration, such as dehydration that results from strenuous exercise.

The compositions for use and for formulation of a medicament are prepared for administered a an amount of copolymer that is sufficient to produce a circulating amount of the polyoxyethylene/polyoxypropylene copolymer of from 0.05 mg/mL to 10 mg/mL, such as from 0.2 mg/mL to 4.0 mg/mL, or about 0.5 mg/mL to 1.5 mg/mL, and particularly 0.5 mg/mL to 1.5 mg/mL. The poloxamer can be formulated at a concentration ranging from about 10.0 mg/mL to about 300.0 mg/mL or 10.0 to 200.0 mg/mL, such as at or at least 10.0, 15.0, 20.0, 25.0, 30.0, 35.0, 40.0, 45.0, 50.0, 55.0, 60.0, 65.0, 70.0, 75.0, 80.0, 85.0, 90.0, 95.0, 100.0, 105.0, 110.0, 115.0, 120.0, 125.0, 130.0, 135.0, 140.0, 145.0, 150.0, 155.0, 160.0, 165.0, 170.0, 175.0, 180.0, 185.0, 190.0, 195.0 or 200.0 mg/mL, for direct administration. Typically, the concentration is not more than 22.5%, i.e., 225 mg/mL.

The composition is formulated at suitable concentration so that it can be administered by IV, such as continuous infusion or bolus injection, to produce the target circulating concentration. Exemplary concentrations of the compositions for use contain a concentration of polyoxyethylene/polyoxypropylene copolymer can range from 10.0 mg/mL to 200.0 mg/mL. Any suitable concentration can be employed. In particular examples, the poloxamer is formulated for administration at a dosage of about or at 25-450 mg/kg, 25-50 mg/kg, 200-450 mg/kg, such as 400 mg/kg subject body weight. Dosage will depend upon the route of administration, and the goal is to achieve the target concentration of at least 0.05 mg/ml, particularly, 0.5 mg/ml to 1.5 mg/ml, for at least several hours, generally at least 12 hours, and up to 72 hours, including 1 day, 2 days, 3 days or 4 days to effect treatment. Typically, the volume to be administered is not greater than 3.0 mL/kg of a subject; the concentration of the composition for use can be readily calculated. The particular volume chosen is one that results in a desired target concentration of poloxamer in the circulation of the subject after administration. Again, the particular volume and dosage is a function of the target circulating concentration, which for treating complications of hemo-concentration is described herein.

In some embodiments of the compositions and uses, the copolymer is formulated for administration by intravenous infusion. In others, the copolymer is formulated for administration by bolus injection. Provided are compositions and uses where treatment is effected in a regimen in which treatment is repeated a plurality of times for at least 12 hours up to 4 days, or at least 12 hours up to 3 days, or at least 1 day to 3 days, such as a regimen of a plurality of treatments with the copolymer. In some embodiments, the copolymer is formulated for administration as a single continuous IV infusion, a plurality of continuous IV infusions, a single IV bolus administration, or a plurality of IV bolus administrations, or a combination thereof. In some embodiments, the composition is for use in treating a non-human subject, such as a non-human mammal.

### BRIEF DESCRIPTION OF THE DRAWINGS

The drawings described herein are for illustrative purposes of selected embodiments and not all possible implementations, and are not intended to limit the scope of the present disclosure.

FIG. 1 is a general process 100 for supercritical fluid extraction (SFE) of a poloxamer.

FIG. 2 is a specific exemplary process 100′ for preparing a poloxamer, such as poloxamer 188, using the methods described herein.

FIG. 3 is a specific exemplary process 100″ for preparing a poloxamer, such as poloxamer 188, using methods described herein.

FIG. 4 shows an extraction apparatus useful in the methods provided herein.

FIG. 5 shows one embodiment of the cross section of stainless spheres of different sizes in a solvent distribution bed.

FIGS. 6A-6B shows a GPC comparison of low molecular weight substance content in a commercially available poloxamer 188 (FIG. 6A) versus a material purified according to an embodiment provided herein (FIG. 6B).

FIG. 7 shows a GPC of long circulating material free (LCMF) poloxamer 188 purified according to an embodiment of the methods provided herein.

FIGS. 8A-8B shows enlarged HPLC-GPC chromatograms depicting the molecular weight distribution of components in plasma over time.

FIGS. 9A-9B shows individual plasma concentrations of poloxamer 188 (FIG. 9A) and high molecular weight component (FIG. 9B) in healthy humans during and following a 48 hour continuous IV infusion of purified poloxamer 188 as described in Grindel et al. (Biopharmaceutics & Drug Disposition (2002) 23:87-103).

### DETAILED DESCRIPTION

Outline

A. Definitions

B. Side effects and complications of dehydration and diuresis
1. Hemo-concentration
2. Diuretic therapy
3. Diuresis and side effects/complications
C. Treatment of the side effects of diuresis, dehydration and/or hemo-concentration by administration of a polyoxyethylene/polyoxypropylene copolymer
   1. Poloxamers for preventing and treating complications from hemo-concentration
   2. Poloxamer 188
      3. Longwe circulating material free (LCMF) poloxamer
      4. Supercritical fluid extraction methods to purify poloxamers
         a. Process for extraction
         b. Extraction vessel and system
         c. Extraction and removal of extractants
         d. Exemplary methods
i. Removal of low molecular weight (LMW) components
ii. Preparation of longer circulating material free (LCMF) poloxamer
   D. Pharmaceutical compositions and formulations
      1. Formulations
      2. Dosage
      3. Administration
   E. Methods of assessing the side effects of diuresis
      1. Assays of hemo-concentration
      2. Assays of dehydration
   F. Methods of treating the complications of hemo-concentration
      1. Exemplary side effects
         a. Hemo-concentration
         b. Dehydration
      2. Identification of subjects for treatment
         a. Identifying subjects with hemo-concentration
         b. Identifying subjects with dehydration
      3. Monitoring subjects for treatment
   G. Combination treatments
   H. Examples

### A. DEFINITIONS

Unless defined otherwise, all technical and scientific terms used herein have the same meaning as is commonly understood by one of skill in the art to which the invention(s) belong. All patents, patent applications, published applications and publications, Genbank sequences, databases, websites and other published materials referred to throughout the entire disclosure herein, unless noted otherwise, are incorporated by reference in their entirety. In the event that there are a plurality of definitions for terms herein, those in this section prevail. Where reference is made to a URL or other such identifier or address, it understood that such identifiers can change and particular information on the internet can come and go, but equivalent information can be found by searching the internet. Reference thereto evidences the availability and public dissemination of such information.

As used herein, “diuresis” refers to a process resulting in increased production or discharge of urine.

As used herein, “dehydration” refers to a condition or state characterized by excessive loss of water or other fluid, whether from a cell, tissue, organ or body. Loss of water or fluid can be from sweating, urine discharge, fever, vomiting, diarrhea and can result from diuresis. Typically, dehydration occurs when more water and fluids exit the body than enter the body.

As used herein, hemo-concentration refers to increased concentrations of cellular and non-cellular components of blood. It can results from either (a) an increase in the amount of cellular and non-cellular components of the blood in a constant volume of blood; or (b) a constant amount of cellular and non-cellular components of the blood in a decreasing volume of blood. Hemo-concentration is an increase in the concentration of red blood cells and plasma proteins.

As used herein, microvascular hemodynamic dysfunction refers to impaired or reduced blood flow in the microvasculature, which includes the arterioles, capillaries and venules.

As used herein, a “polyoxyethylene/polyoxypropylene copolymer,” “PPC” or “poloxamer” refers to a block copolymer containing a central block of polyoxypropylene (POP) flanked on both sides by blocks of polyoxyethylene (POE) having the following molecular formula:

HO(C₂H₄O)_{a′}—[C₃H₆O]_{b}—(C₂H₄O)ₐH

Poloxamers are polyoxyethylene/polyoxypropylene copolymers defined by this POE-POP-POE structural motif. Specific poloxamer further are defined by the number of repeating POE and POP units, which provide specific poloxamers with different chemical and physical characteristics, as well as pharmacodynamic properties. In general for purposes herein, a′ and a can be the same or different and each is an integer, and b is an integer. Exemplary poloxamers having the general formula described above include poloxamers where a or a′ is an integer 5-150, and b is an integer 15-75, such as poloxamers where a is an integer 70-105, and b is an integer 15-75. The nomenclature of the polyoxyethylene/polyoxypropylene copolymer relates to its monomeric composition. The first two digits of a poloxamer number, multiplied by 100, gives the approximate molecular weight of the hydrophobic polyoxypropylene block. The last digit, multiplied by 10, gives the approximate weight percent of the hydrophilic polyoxyethylene content. For example, poloxamer 188 describes a polymer containing a polyoxypropylene hydrophobe of about 1,800 Da with the hydrophilic polyoxyethylene content being about 80% of the total molecular weight. Poloxamers often are synthesized in two steps, first by building the polyoxypropylene core, and then by addition of polyoxyethylene to the terminal ends of the polyoxypropylene core. Because of variation in the polymerization during both steps, a poloxamer typically contains heterogenous polymer species that vary primarily in molecular weight. Various truncated polymer chains and unreacted monomers also can be present. The distribution of polymer species can be characterized using standard techniques known to a skilled artisan, including, but not limited to, gel permeation chromatography (GPC), colligative property measurements, light scattering techniques and viscometry.

As used herein, “polydispersity” or D refers to the heterogeneity of the size distribution of material in a particular sample of a polymer composition. A monodisperse sample is one in which all material is of identical size. In such a case, the polydispersity value is 1. A typical polymer has a range of 2 to 5. Some polymers have a polydispersity in excess of 20. Hence, a large polydispersity value indicates wide variation in the size of material in a particular sample, while a lower polydispersity value indicates less variation. Methods for assessing polydispersity are known in the art, and include methods as described in U.S. Pat. No. 5,696,298. For example, polydispersity can be determined from GPC chromatograms. It is understood that polydispersity values can vary depending on the particular chromatogram conditions, the molecular weight standards and the size exclusion characteristics and number of GPC columns and analytical software employed. It is within the level of a skilled artisan to convert any polydispersity value that is obtained using different conditions, standards, columns and software to the values described herein by running a single sample on both systems and then comparing the polydispersity values from each chromatogram.

As used herein, “poloxamer 188” refers to a polyoxyethylene/polyoxypropylene copolymer or poloxamer that has the following molecular formula:

HO(C₂H₄O)_{a′}—[C₃H₆O]_{b}—(C₂H₄O)ₐH, where:

each of a and a′ is an integer such that the hydrophile portion represented by (C₂H₄O) (i.e., the polyoxyethylene portion of the copolymer) constitutes approximately 60% to 90%, such as approximately 70%-90%, or 80% or 81% of the total molecular weight; and b is an integer such that the hydrophobe, represented by (C₃H₆O), has a molecular weight of approximately 1,300 to 2,300 Da, such as 1,400 to 2,000 for example approximately 1,750 Da. For example, a is about 79 or 80, and b is approximately or is 27 or 28. The average total molecular weight of the compound depends upon the particular sample, but is in the range 7500-9500 or 7680 to 9510 Da, and can be 8,400-8,800 Da, for example about or at 8,400 Da. Poloxamer 188 is commercially available, such as a poloxamer sold under trademarks that include Pluronic®, Kolliphor®, Lutrol®. The compositions of these poloxamers, and any such preparation, can vary. Side reactions that occur during the synthesis of poloxamer 188 generate other material, which present in any particular sample of a poloxamer 188 preparation. This other material includes, among other things, polymer species that differ in size, composition and poly(oxyethylene) to poly(oxypropylene) ratio (e.g., different numbers of repeating units of oxypropylene and oxyethylene), such as diblock polymers, unsaturated polymers, oligomeric glycols, including oligo(ethylene glycol) and oligo (propylene glycol); and poloxamer degradation products, including alcohols, aldehydes, ketones, and hydroperoxides. Accordingly, any particular sample of poloxamer 188 contains a heterogenous distribution of poloxamer-type polymer species and other material, which can be observed through analytical methods, such as GPC. In such preparation, there is a main peak and a “shoulder” peak on each side of the main peak, representing low molecular weight and high molecular weight material, respectively. In some preparations the low molecular weight shoulder material has been removed.

As used herein, “main peak” refers largest peak in a preparation. Its exact molecular weight range in a P188 sample depends upon the particular sample and preparation methods. The skilled artisan will recognize such peak. Typically the molecular weight range is 7680 to 9510 or 7,750 to 9,250 Da, for example about or at 8,400-8,800, such as 8,400 or 8,500 Da. For example for the P188 preparation described in U.S. Pat. No. 5,696,298, the main peak species include those that elute by gel permeation chromatography (GPC) between 14 and 15 minutes.

As used herein, “low molecular weight” or “LMW” refers to material in a particular sample of a poloxamer 188 that has a molecular weight generally less than 7,000 Da, less than 6,000 Da, less than 5,500 Da, less than 4500 Da or less. The molecular weight range is lower than the main peak. For example, LMW materials have a molecular weight between 2,300 daltons to 5,000 daltons. For example, LMW species in the preparation described in U.S. Pat. No. 5,696,298 are those that elute by gel permeation after 15 minutes.

As used herein, “high molecular weight” or “HMW” refers to material in a particular sample of poloxamer 188 that has a molecular weight generally greater than 13,000 Da, such as greater than 14,000 Da, greater than 15,000 Da, greater than 16,000 Da or greater. The molecular weight range is higher than the main peak. For example, in the preparation described in U.S. Pat. No. 5,696,298, HMW species include those that elute by gel permeation chromatography (GPC) at between 13 and 14 minutes.

As used herein, “purified poloxamer 188” refers to a poloxamer 188 that has polydispersity value of less than or about 1.07, such as less than or about 1.05 or less than or about 1.03. For example, the poloxamer 188 is purified to remove or reduce low molecular weight components. An exemplary purified poloxamer 188 is described in U.S. Pat. No. 5,696,298.

As used herein, reference to a poloxamer 188 in which “low molecular weight material has been removed” or “low molecular weight material has been reduced,” or similar variations thereof, refers to a sample of poloxamer 188 in which the LMW material is no more than or less than 3.0%, and generally no more than or less than 2.0% or no more than or less than 1.5% of the total material in the sample. For example, it is a sample of poloxamer where the material that is less than 4,500 Da is no more than 1.5% of the total material in the sample. Typically, such a poloxamer 188 exhibits reduced toxicity compared to forms of poloxamer 188 that contain a higher or greater percentage of low molecular weight material.

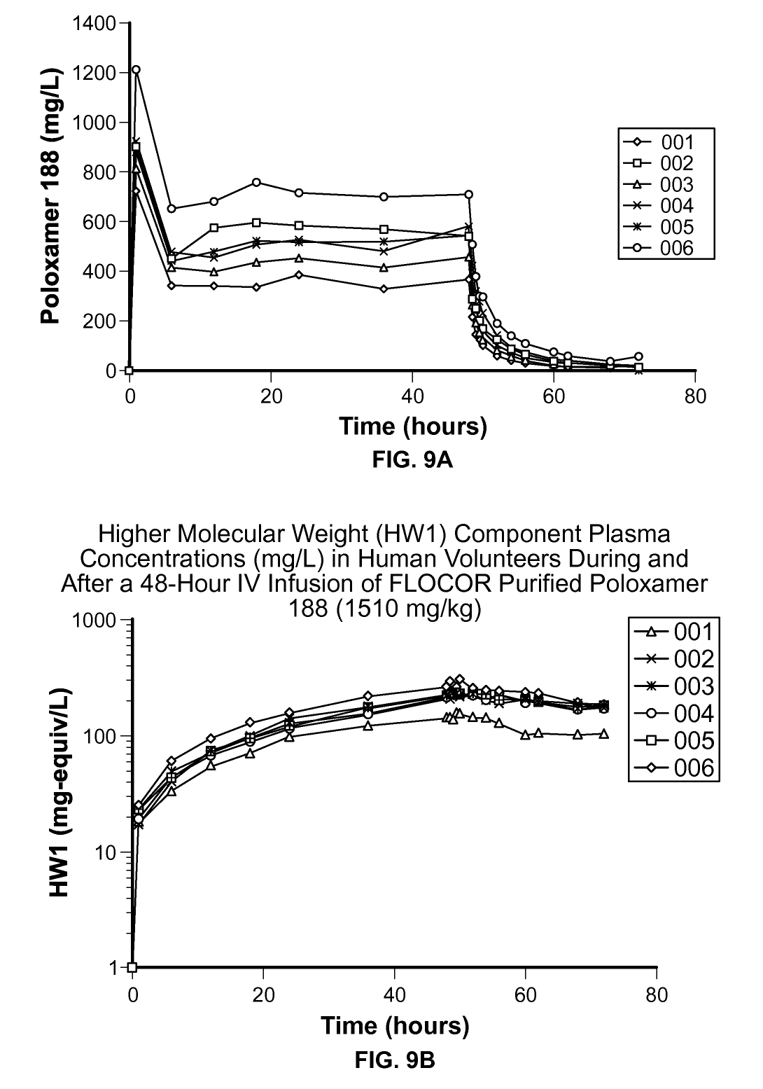

As used herein, “longer circulating material free poloxamer” or “LCMF poloxamer” refers to a purified poloxamer 188 preparation that, additionally, does not contain any material which, when administered to a subject, is or gives rise to a component that has a substantially or considerably longer residence time in the circulation (i.e., a longer half-life) than the main peak. Briefly, purified poloxamer 188 known in the art (see Grindel et al. (2002) Journal of Pharmaceutical Sciences, 90:1936-1947 or Grindel et al. (2002) Biopharmaceutics & Drug Disposition, 23:87-103) contains materials that, when administered to a subject, shows two peaks upon GPC analysis of the subject's plasma samples, with each peak having a different pharmacokinetic profile exemplified by markedly different half-lives (i.e. rates of clearance from the circulation). The main peak exhibits a half-life of about 7 hours, while the second peak (with a higher average molecular weight) exhibits a half-life of approximately 70 hours or more, an approximately 10-fold or more increase in half-life (compared to the main peak) and, thus, a substantially longer residence time in the circulation (see, e.g., FIG. 9A and FIG. 9B). Thus, an LCMF poloxamer is a purified poloxamer 188 that does not contain any material that, when administered to a subject, is or gives rise to a material with a half-life that is more than 5.0-fold greater than the half-life of the main peak, and generally no more than 4.0, 3.0, 2.0 or 1.5 fold greater than the half-life of the main peak. Typically, an LCMF poloxamer is a purified poloxamer in which the components of the polymeric distribution clear from the circulation at approximately the same rate. In particular examples, an LCMF poloxamer is a purified poloxamer 188 in which the material that is greater than 13,000 daltons is no more than or is less than 1%, such as less than 0.9%, less than 0.8%, less than 0.7%, less than 0.6%, less than 0.5% or less of the total material in the sample, that a living body requires to eliminate one half of the quantity of an administered substance through its normal channels of elimination. The normal channels of elimination generally include the kidneys and liver in addition to other excretion pathways (e.g. respiration). A half-life can be described as the time it takes the concentration of a substance to halve its concentration from steady state or from a certain point on the elimination curve. A half-life typically is measured in the plasma and can be determined by giving a single dose of drug, and then measuring the concentration of the drug in the plasma at various times to determine the relationship between time and decline in concentration as the substance is eliminated. For example, the concentration of a poloxamer (or its metabolites or components), and so their respective half-lives, can be determined as described herein by quantifying the plasma level of the various material in a subject using HPLC-GPC methods as described herein. Briefly, the height of the eluting HPLC-GPC peak is compared to a reference standard of known concentration to quantify the material in the subject's plasma Studies to determine the half-life of a substance are readily carried out by those skilled in the art.

As used herein Cmax refers to the peak plasma concentration of a drug after administration.

As used herein, “impurities” refer to unwanted material in a poloxamer preparation. When analyzed by GPC, impurities typically include material that is not part of the main peak, or is part of the main peak but where the size, composition and poly(oxyethylene) to poly(oxypropylene) ratio of the material is not desired, and, with respect to poloxamer 188 and purified poloxamer 188, can include material with a molecular weight less than 4,500 daltons and/or a molecular weight greater than 13,000 daltons.

As used herein, “remove” or “reduce” with reference to material in a poloxamer preparation refers to decreasing the weight percentage of the material relative to the initial weight percentage of the material. Generally, a reduction involves a decrease by at least 1%, and typically at least 2%, 3%, 4%, 5%, or more. For example, most commercial preparations of poloxamer 188 contain LMW material (less than 4,500 daltons) that is about 4% (by weight) of all material in the preparation. The LMW material is considered reduced in a purified product if there is 3% or less (by weight) of the LMW material following purification, such as 3%, 2% or less or 1% or less.

As used herein, “solvent” refers to any liquid in which a solute is dissolved to form a solution.

As used herein, a “polar solvent” refers to a solvent in whose molecules there is either a permanent separation of positive and negative charges, or the centers of positive and negative charges do not coincide. These solvents have high dielectric constants, are chemically active, and form coordinate covalent bonds, Examples are alcohols and ketones.

As used herein, “feed” refers to a solute dissolved in a solvent.

As used herein, an “extraction solvent” refers to any liquid or supercritical fluid that can be used to solubilize undesirable materials that are contained in a poloxamer preparation. It is a solvent that can effect solvent extraction to separate a substance from one or more others based on variations in the solubilities. Generally an extraction solvent is immiscible or partially miscible with the solvent in which the substance of interest is dissolved. For example, an extraction solvent is one that does not mix or only partially mixes with a first solvent in which the substance of interest is dissolved, so that, when undisturbed, two separate layers forms. Exemplary extraction solvents are supercritical liquids or high pressure liquids.

As used herein, the terms “supercritical liquid” and “supercritical fluid” include any compound, such as a gas, in a state above its critical temperature (T_{c}; i.e. the temperature, characteristic of the compound, above which it is not possible to liquefy the compound) and critical pressure (p_{c}; i.e., the minimum pressure which would suffice to liquefy the compound at its critical temperature). In this state, distinct liquid and gas phases typically do not exist. A supercritical liquid typically exhibits changes in solvent density with small changes in pressure, temperature, or the presence of a co-modifier solvent.

As used herein, “supercritical carbon dioxide” refers to a fluid state of carbon dioxide where it is held at or is above its critical temperature (about 31° C.) and critical pressure (about 74 bars). Below its critical temperature and critical pressure, carbon dioxide usually behaves as a gas in air or as a solid, dry ice, when frozen. At a temperature that is above 31° C. and a pressure above 74 bars, carbon dioxide adopts properties midway between a gas and a liquid, so that it expands to fill its container like a gas but with a density like that of a liquid.

As used herein, “critical temperature” or “critical point” refers to the temperature that denotes the vapor-liquid critical point, above which distinct liquid and gas phases do not exist. Thus, it is the temperature at and above which vapor of the substance cannot be liquified no matter how much pressure is applied. For example, the critical temperature of carbon dioxide is about 31° C.

As used herein, “critical pressure” refers to the pressure required to liquefy a gas at its critical temperature. For example, the critical pressure of carbon dioxide is about 74 bars.

As used herein, the term “high pressure liquid” includes a liquid formed by pressurizing a compressible gas into the liquid at room temperature or a higher temperature.

As used herein, a “co-modifier solvent” refers to a polar organic solvent that increases the solvent strength of an extraction solvent (e.g. supercritical fluid carbon dioxide). It can interact strongly with the solute and thereby substantially increase the solubility of the solute in the extraction solvent. Examples of a co-modifier solvent include alkanols. Typically between 5% and 15% by weight of co-modified solvent can be used.

As used herein, the term “alkanol” includes simple aliphatic organic alcohols. In general, the alcohols intended for use in the methods provided herein include six or fewer carbon atoms (i.e., C₁-C₆ alkanols). The alkane portion of alkanol can be branched or unbranched. Examples of alkanols include, but are not limited to, methanol, ethanol, isopropyl alcohol (2-propanol), and tert-butyl alcohol.

As used herein, “subcritical extraction” refers to processes using a fluid substance that would usually be gaseous at normal temperatures and pressures that is converted to liquids at higher pressures and lower temperatures. The pressures or temperatures are then normalized and the extracting material is vaporized leaving the extract. Extractant can be recycled.

As used herein, “extraction vessel” or “extractor” refers to a high-pressure vessel that is capable of withstanding pressures of up to 10,000 psig and temperatures of up to 200° C. The volume of the vessels can range from 2 mL to 5,000 L or larger, and generally 1 L to 1,000 L, such as 5 L to 500 L, and can be 1 L to 200 L, such as 5 L to 150 L. Extraction vessels generally are made out of stainless steel. Such devices are well known to a skilled artisan and available commercially.

As used herein, isocratic refers to a system in which an extraction solvent is used at a constant or near constant concentration.

As used herein, “gradient” or “gradient steps” refers to a system in which two or more extraction solvents are used that differ in its composition of components, typically by changes in concentration of one or more components. For example, the concentration of the alkanol solvent (e.g. methanol) is successively increased during the course of the extraction. Thus, the extraction solvent does not remain constant.

As used herein, “plurality” refers to a number of iterations of a process or step. A plurality is 2 or more. The number of repeats can be 2, 3, 4, 5, 6 or more.

As used herein, “extracted material” refers to the product containing the removed materials.

As used herein, “raffinate” refers to a product which has had a component or components reduced or removed. The product containing the removed material is the extract.

As used herein, “batch method” or “batch extraction” refers to a process of extracting the solute from one immiscible layer by shaking the two layers until equilibrium is attained, after which the layers are allowed to settle before sampling. For example, a batch extraction can be performed by mixing the solute with a batch of extracting solvent. The solute distributes between the two phases. Once equilibrium is attained, the mixing is stopped and the extract and raffinate phases are allowed to separate. In this method, the spent solvent can be stripped and recycled by distillation or fresh solvent can be added continuously from a reservoir.

As used herein, a “continuous method” or “continuous extraction” refers to a process in which there is a continuous flow of immiscible solvent through the solution or a continuous countercurrent flow of both phases. For example, a continuous extracting solvent is mixed with the solute. The emulsion produced in the mixer is fed into a settler unit where phase separation takes place and continuous raffinate and extract streams are obtained.

As used herein, a single infusion refers to an infusion that provides an effective dosage amount of a compound or pharmaceutical composition in only one infusion or administration.

As used herein, a pharmaceutical composition that contains a poloxamer refers to a product containing a polyoxyethylene/polyoxypropylene copolymer or poloxamer, as described herein, such as an LCMF poloxamer, formulated with one or more pharmaceutically acceptable excipients. In certain instances, the pharmaceutical composition contains an aqueous injectable solution of the poloxamer buffered at a desired pH, such s 6-7 or 6 or about 6, with a buffering agent. Exemplary of such buffering agents are any known to those of skill in the art to be biocompatible, such as citrate, including for example sodium citrate and/or citric acid. Suitable concentrations can be empirically determined, but typically range from 0.005 to 0.05 M, particularly about 0.01 M in an isotonic solution such as saline. In certain instances, pharmaceutical compositions useful in the methods herein are known to those of skill in the art for formulating poloxamer (see, e.g., International PCT application publication no. WO 94/08596 and other such references and publications herein).

As used herein, “treating” or “treatment” of a subject having a disease, disorder, condition or dysfunction refers to providing the subject an effective amount of a compound or pharmaceutical composition. Hence treatment encompasses prophylaxis, therapy and/or cure. Treatment also encompasses any pharmaceutical use of the compounds and pharmaceutical compositions herein. Treating results in ameliorating or reducing symptoms associated with a disease or condition. Treatment means any manner in which the symptoms of a condition, disorder or disease are ameliorated or otherwise beneficially altered.

As used herein, amelioration of the symptoms of a particular disease or disorder by a treatment, such as by administration of a pharmaceutical composition or other therapeutic, refers to any lessening, whether permanent or temporary, lasting or transient, of the symptoms that can be attributed to or associated with administration of the composition or therapeutic.

As used herein, prevention or prophylaxis refers to methods in which the risk of developing disease or condition is reduced. Prophylaxis includes reduction in the risk of developing a disease or condition and/or a prevention of worsening of symptoms or progression of a disease or reduction in the risk of worsening of symptoms or progression of a disease.

As used herein an “effective amount” of a compound or pharmaceutical composition is an amount that is (a) sufficient to improve in some manner how the subject feels, functions or survives (e.g. to reduce symptoms); (b) sufficient to achieve a desired physiological effect; and/or (c) sufficient to provide some other benefit: in each case, whether the improvement, effect, or benefit is permanent, lasting, temporary, periodic, transitory or otherwise. Such amount can be administered as a single dose or can be administered according to a dose schedule or regimen (e.g. repeat doses, continuous dosing), whereby it improves how the subject feels, functions or survives, and/or achieves a desired physiologic and/or provides other benefit. For example, the effective amount of a poloxamer or pharmaceutical composition described herein is an amount that, when administered to a human or non-human subject treats the diuresis. Such amounts are described herein below, and are less in volume than the volume of fluid loss, since the poloxamer is not administered as blood substitute, but for its ability to ameliorate the adverse effects of dehydration and diuresis.

As used herein, “subject” refers to any animal, regardless of class, order, family, genus or species (or any subcategory), such as, but not limited to: hominidae (such as humans); non-human primates (such as chimpanzees, gorillas and monkeys); rodentia (such as mice, rats, hamsters and gerbils); ruminants (such as goats, cows, deer, sheep); suidae (such as pigs); bovidae (such as bison); equus (such as horses); canidae (such as dogs); felidae (such as cats); in all cases, whether or not domesticated. Thus, a “subject” to be treated includes humans or non-human animals.

As used herein, a combination refers to any association between two or among more items. The association can be spatial, such as in a kit, or refer to the use of the two or more items for a common purpose.

As used herein, a composition refers to any mixture of two or more products or compounds. It can be a solution, a suspension, liquid, powder, a paste, aqueous or non-aqueous formulations or any combination thereof.

As used herein, an “article of manufacture” is a product that is made and sold. As used throughout this application, the term is intended to encompass modified protease polypeptides and nucleic acids contained in articles of packaging.

As used herein, fluid refers to any composition that can flow. Fluids thus encompass compositions that are in the form of semi-solids, pastes, solutions, aqueous mixtures, gels, lotions, creams and other such compositions.

As used herein, a “kit” refers to a packaged combination, optionally including reagents and other products and/or components for practicing methods using the elements of the combination. Kits optionally include instructions for use.

As used herein, “erythrocyte sedimentation rate” is a measurement of the sedimentation rate of erythrocytes (e.g., red blood cells) in a period of one hour. Typically, anticoagulated blood is placed in a Westergren tube and the rate at which the erythrocytes fall in an hour is measured as millimeters per hour (mm/h). The rate can be used as an indirect measurement of the presence of inflammation or inflammatory disorders/diseases. It also can be used as an indirect measurement of the formation of aggregates of red blood cells and/or “sludged” blood as a result of hemo-concentration (e.g., increased levels of pro-aggregation factors, such as fibrinogen).

As used herein, “diuretic” refers to any compound or substance that assists in diuresis. Diuretics can promote the production or discharge of urine. Examples of a diuretic include, but are not limited to, a loop diuretic such as furosemide (sold under the trademark Lasix™, Frumex), ethyacrynic acid (sold under the trademark Edecrin®), bumetanide and torasemide (sold under the trademark Demadex®), a thiazide such as chlorothiazide, bendroflumethiazide, hydrochlorothiazide (sold under the trademark Microzide®), metolazone (sold under the trademark Zaroxolyn®), and indapamide, and a potassium-sparing diuretic such as spironolactone (sold under the trademark Aldactone®), eplerenone (sold under the trademark Inspra®), amiloride, and triamterene (sold under the trademark Dyrenium®), or an osmotically active agent, such as mannitol.

As used herein, “approximately” and “about” modify a numerical value, indicating a close range around that explicit value. If “X” were the value, “about X” would indicate a value from 0.9X to 1.1X, and a value from 0.95X to 1.05X. Any reference to “about X” specifically indicates at least the values X, 0.95X, 0.96X, 0.97X, 0.98X, 0.99X, 1.01X, 1.02X, 1.03X, 1.04X, and 1.05X. Thus, “about X” is intended to include the value “0.98X.”

As used herein, “bolus” refers to a drug administration where a certain dose is administered over a relatively short period of time. Generally, a bolus is administered over a period of time less than 60 minutes.

As used herein, “continuous infusion” refers to a drug administration where a certain dose is administered over a relatively longer period of time. Generally, a continuous infusion is administered for a period of time greater than one hour such as 12 hours or 24 hours.

As used herein, “acute phase reactant” refers to a group of molecules that are physiologically active and, typically, rapidly increase in concentration in the circulation as part of an inflammatory response. Prominent acute phase reactants include, but are not limited to, fibrinogen, serum amyloid A, c-reactive protein, complement factor, prothrombin, plasminogen and Von Willebrand factor.

As used herein, the singular forms “a,” “an” and “the” include plural referents unless the context clearly dictates otherwise. Thus, for example, reference to compound, comprising “an extracellular domain” includes compounds with one or a plurality of extracellular domains.

As used herein, ranges and amounts can be expressed as “about” or “approximately” a particular value or range. About also includes the exact amount. Hence “about 0.05 mg/mL” means “about 0.05 mg/mL” and also “0.05 mg/mL.”

As used herein, “optional” or “optionally” means that the subsequently described element, event or circumstance does or does not occur, and that the description includes instances where said event or circumstance occurs and instances where it does not. For example, an optionally substituted group means that the group is unsubstituted or is substituted.

As used herein, the abbreviations for any protective groups, amino acids and other compounds, are, unless indicated otherwise, in accord with their common usage, recognized abbreviations, or the IUPAC-IUB Commission on Biochemical Nomenclature (see, (1972) Biochem. 11:1726).

### B. SIDE EFFECTS AND COMPLICATIONS OF HEMO-CONCENTRATION FROM DEHYDRATION AND DIURESIS OR OTHER CAUSES

Provided are methods of treating or ameliorating or preventing the side effects and complications that result from hemo-concentration of blood. Hemo-concentration can result from dehydration and/or diuresis, including diuretic-induced diuresis. Treatment is effected by administering to a subject exhibiting symptoms of or having dehydration and/or diuresis, a polyoxyethylene/polyoxypropylene copolymer (poloxamer), as described herein. Administration of the polyoxyethylene/polyoxypropylene copolymer can treat the complications and also can prevent (reduce the risk) of the complications or the severity of the complications, including the side effects of diuretics.

1. Hemo-Concentration

Hemo-concentration results from an increase in concentrations of blood components, including cells and proteins. Concentrations can increase from fluid loss and/or increased numbers of cells and/or proteins. This concentration of blood components can have complications. Exemplary of hemo-concentration is the hemoconcentration that results from diuresis as a consequence of treatment with diuretics or as consequence or symptom of particular diseases. Hemo-concentration can occur from dehydration, including from diuresis and from loss of fluids, such as from strenuous exercise.

In subjects experiencing dehydration, whether from diuresis or other cause, there is a hemo-concentration. As a result the concentration of blood components increases. This includes an increase in the concentration of fibrinogen and (other hydrophobic proteins) and red blood cells (RBC) and other cells. The hemo-concentration results in the formation of RBC aggregates, by interactions including a “bridging” interaction between fibrinogen and RBC. When these aggregates form in the blood flow becomes “sludged” especially in the microcirculation.

In the methods and uses herein, administration of a poloxamer, a polyoxyethylene/polyoxypropylene copolymer, such as a P188, as described herein and known to those of skill in the art, is administered. The poloxamer, among other effects can reduce the complications. The poloxamer, for example can antagonize the bridging interaction between the fibrinogen and RBC. The poloxamer is not administered to simply dilute the blood, but rather has an effect on the blood to reduce the complications/side effects of hemo-concentration.

As described in the sections that follow, an effective amount of a poloxamer composition is administered. The suitable dosage achieves a blood concentration that ameliorates the symptoms. There are many possible dosing regimens; the goal is to the an effective plasma concentration for a time sufficient to effect treatment of a subject. The particular dosage regimen will depend upon the subject, the severity and nature of the side effects of the diuresis or dehydration. The skilled physician can select an appropriate regiment.

In particular, the methods include administration of a poloxamer, a polyoxyethylene/polyoxypropylene copolymer, such that the administration is sufficient to result in a concentration of the poloxamer in the circulation of the subject of from at or about 0.05 mg/mL to at or about 15 mg/mL, for example, from at or about 0.2 mg/mL to at or about 4.0 mg/mL, such as at or about at least 0.5 mg/mL. The concentration of the poloxamer in the circulation of the subject can be representative of a single time point or representative of a mean steady state concentration that is maintained for a period of time, for example, up to 72 hours or more after administration or by virtue of multiple doses.

Typically, an optimal steady-state plasma concentration range for treatment of side effects of diuresis/dehydration or hemo-concentration is a plasma concentration in circulation of about 0.5-1.5 mg/ml or 0.5-1.5 mg/ml for a time sufficient to effect treatment. Treatment typically lasts for 12 hours to several days, such as 1, 2, 3 or 4 days. The poloxamer can be administered by any suitable route and way of administration. Typically it can be administered by intravenous (I.V.) infusion or bolus. For example, a concentration of 0.5 mg/ml can be maintained by giving an IV infusion of 50 mg/kg/hr; a plasma concentration of 1.0 mg/ml can be maintained by administering 100 mg/kg/hr. In general, for treatment, the infusion can be continued for between 12-48 hours as needed. Alternatively, repeat bolus administrations can be administered. For example, 50 mg/kg as an IV bolus every 6 hours over 1-3 or 4 days can be administered to achieve the a plasma concentration of about 0.5 mg/ml. to achieve a higher plasma concentration of about 1 mg/ml 0.100 mg/kg every 6 hours for 1-3 or 4 days would result in concentrations in the middle of the desired range.

The methods provided herein can, be used in the treatment of any side effect or consequence associated with diuresis or dehydration, caused by diuretics or other treatments or conditions that result in hemo-concentration. These side-effects include, but not limited to, electrolyte imbalance, dehydration, arrhythmia, alterations of plasma volume, hemo-concentration of blood plasma proteins and/or blood cells, microvascular hemodynamic dysfunction, and any other side effect or unwanted consequence associated with the increased diuresis.

In particular, the methods provided herein can be used in the treatment of subjects in which there is an increased level of blood cells, especially red blood cells, and plasma proteins in the blood, such as subjects with impaired circulation, particularly microcirculation. Subjects are any that have diuresis or dehydration or hemo-concentration from other causes. Subjects include those treated with diuretics, endurance athletes, subjects exposed to prolonged heat exposure, subjects with cardiovascular disorders such as atherosclerosis, diabetes, heart failure, arteritis, raynauds, sickle cell disease, polycythemia, post-surgical patients, including transplant patients. Subjects also include those with dehydration, such as from very strenuous exercise and exposure to high temperatures or disorders or condition in which heat can be lost by evaporation or sweating

In some of the methods provided herein, administration of the poloxamer is in combination with or subsequent to therapies for underlying conditions or diuretic therapy. Exemplary of the treatments or conditions that lead to dehydration or diuresis or hemo-concentration is diuretic therapy. It is understood that the methods herein can be used treatment of any side-effects resulting from hemo-concentration or dehydration, such as any condition or treatment or combination thereof that results in a loss of the body's fluid(s) or an increase in blood components.

2. Diuretic Therapy

Hemo-concentration can result from therapy with diuretics. Diuretics are a class of drugs that are administered to treat or ameliorate a variety of medical conditions, including, but not limited to, kidney and liver related conditions, high blood pressure (i.e., hypertension), glaucoma, increased intra-ocular pressure, and heart-related conditions, such as congestive heart failure. Diuretic therapy is employed to restore and maintain a normal fluid volume in patients with clinical evidence of excess fluid, typically demonstrated by congestive symptoms (orthopnea, edema, and shortness of breath), or signs of elevated filling pressures (jugular venous distention, peripheral edema, pulsatile hepatomegaly, and, less commonly, rales).

Diuretic therapy results in a decrease of the body's fluid volume and venous pressure due to an increase in renal excretion of water and solutes, mainly sodium. Additionally, diuretics serve to adjust the body's water and electrolyte balance. For most diuretics, these effects are due to an inhibition or reduction of sodium (Na⁺) and water reabsorption by the nephrons of the kidney. This action increases the renal excretion of Na⁺ and water out of the body, thus decreasing the extracellular fluid (ECF) volume. Typically, sodium enters the ECF via the diet and is excreted in almost identical amounts in the urine. In normal adults, more than 99% of the sodium that enters the nephrons of the kidneys, via glomerular filtration, is transported out of the tubular fluid (i.e., fluid in the tubules of the kidney) back into the ECF. Salt retention occurs when the level of sodium excretion falls below the level of sodium intake. Administration of one or more diuretics treats this imbalance by reducing the Na⁺ and water reabsorption by the kidneys, thus increasing their excretion in the urine. Certain diuretics suppress sodium and water reabsorption by inhibiting the function of specific proteins that are responsible for the transport of electrolytes across the epithelial membrane, while others inhibit water and sodium reabsorption by increasing intratubular osmotic pressure. Different types of diuretics can inhibit different transporters in different segments of the tubular system.

Diuretics are divided into classes, distinguished by the location of the kidney at which sodium reabsorption is impaired by the diuretic. Major classes of diuretics include loop diuretics, thiazide-type diuretics, potassium-sparing diuretics, osmotic diuretics and carbonic anhydrase inhibitors.

Loop diuretics, or high-ceiling diuretics, act on the sodium-potassium-chloride cotransporter in the thick ascending limb of the loop of Henle within the kidney, inhibiting electrolyte reabsorption and resulting in the excretion of sodium, as well as potassium, calcium and magnesium. This transporter normally reabsorbs about 25% of the sodium load; therefore, inhibition of this pump can lead to a significant increase in the distal tubular concentration of sodium, reduced hypertonicity of the surrounding interstitium, and less water reabsorption in the collecting duct. This altered handling of sodium and water leads to both diuresis (increased water loss) and natriuresis (increased sodium loss). By acting on the thick ascending limb, which handles a significant fraction of sodium reabsorption, loop diuretics are very powerful diuretics. Exemplary loop diuretics include ethacrynic acid, furosemide, bumetanide, and torasemide (or torsemide).

Thiazide diuretics, the most commonly used type of diuretic, act in the distal tubule and connecting segment of the kidneys by inhibiting the sodium-chloride transporter in the distal tubule. This transporter typically only reabsorbs about 5% of filtered sodium, thus thiazide diuretics are less effective than loop diuretics in producing diuresis and natriuresis. Thiazide diuretics include chlorothiazide, chlorthalidone, hydrochlorothiazide, hydroflumethiazide, indapamide, methyclothiazide, metolazone, and polythiazide. Thiazide diuretics can induce hyperglycemia and aggravate pre-existing diabetes mellitus, and also can cause increased serum cholesterol, low-density lipoprotein (LDL) and triglyceride concentration.

Another class of diuretics are the potassium-sparing diuretics, which can act through one of several mechanisms. Unlike loop and thiazide diuretics, which are considered “potassium-wasting” diuretics, some of these diuretics do not act directly on sodium transport. Some potassium-sparing diuretics antagonize the actions of aldosterone (i.e., aldosterone receptor antagonists) at the distal segment of the distal tubule, causing more sodium and water to pass into the collecting duct and be excreted in the urine. Others directly inhibit sodium channels associated with the aldosterone-sensitive sodium pump, and therefore have similar effects on potassium and hydrogen ion as the aldosterone antagonists. Potassium-sparing diuretics include steroidal compounds, such as spironolactone and eplerenone, and non-steroidal compounds, such as triamterene and amiloride. Whereas spironolactone increases calcium excretion, triamterene and amiloride cause an increase in sodium and chloride excretion and have little effect on potassium excretion. Common side-effects associated with the use of potassium-sparing diuretics include nausea, stomach cramps, vomiting, diarrhea, leg cramps, dizziness, headache, endocrine imbalances, gynecomastia (abnormal enlargement of one or both breasts in men), altered libido, impotence, hirsutism (excessive body hair), and hyperkalemia (increased serum potassium concentration).

Osmotic diuretics are another class of diuretics. These compounds are poorly reabsorbed by the renal tubules and effect poor net reabsorption of sodium salts. Osmotic diuretics include mannitol, glycerol, urea, and isosorbide.

Another class of diuretic are the carbonic anhydrase inhibitors, such as acetazolamide, dichlorphenamide, and methazolamide. These diuretics inhibit the transport of bicarbonate out of the proximal convoluted tubule into the interstitium, leading to less sodium reabsorption at this site and therefore greater sodium, bicarbonate and water loss in the urine. The carbonic anhydrase inhibitors are the weakest of the diuretics and are mainly used in the treatment of glaucoma.

Diuretics can be administered alone or as a combination of two or more diuretics to increase the effectiveness of either compound alone The reason for this is that one nephron segment can compensate for altered sodium reabsorption at another nephron segment; therefore, blocking multiple nephron sites significantly enhances efficacy.

Diuretics are known and commercially available, including, but not limited to, loop diuretics such as furosemide (sold under the trademark Lasix™, Frumex), ethacrynic acid (sold under the trademark Edecrin®), bumetanide and torasemide (sold under the trademark Demadex®); thiazide diuretics such as chlorothiazide, bendroflumethiazide, hydrochlorothiazide (sold under the trademark Microzide®), metolazone (sold under the trademark Zaroxolyn®), and indapamide; and potassium-sparing diuretics such as spironolactone (sold under the trademark Aldactone®), eplerenone (sold under the trademark Inspra®), amiloride, and triamterene (sold under the trademark Dyrenium®).

For purposes herein, the diuretics can be administered, before, after or concomitant with administration of the polyoxyethylene/polyoxypropylene copolymer

3. Diuresis and Side Effects Thereof

Diuretic drugs inhibit reabsorption of sodium at different segments of the renal tubular system, thus altering how the kidney handles sodium. When the kidney increases the amount of sodium excreted, the amount of water excreted also is increased. Consequently, diuretic therapy increases urine output by the kidneys, i.e., promotes diuresis. Diuresis is a desired effect of diuretic treatment administered to ameliorate or treat a condition such as a kidney or liver related condition, high blood pressure (i.e., hypertension), glaucoma, increased intra-ocular pressure, or a heart-related conditions, such as congestive heart failure. Though diuresis is the desired effect, unwanted consequences of diuresis can occur, including electrolyte imbalance, dehydration, arrhythmia, alterations of plasma volume, and hemo-concentration of blood plasma proteins and blood cells, and combinations thereof. For example, a side effect can include a relative increase in the concentration of plasma proteins such as fibrinogen and/or blood cells such as erythrocytes. In some example, this hemo-concentration is reflected as an elevated erythrocyte sedimentation rate.

Hemoconcentration, can be evidenced by an increasing hematocrit or erythrocyte volume fraction, which represents the volume percentage of red blood cells in blood, can result from diuresis. Diuresis can cause such intravascular volume reduction, which leads to a risk of end-organ hypoperfusion or neurohumoral activation. Indicative of hemo-concentration is a rise in the concentration of red blood cells, such as erythrocytes, and blood plasma proteins including, but not limited to, positive acute phase reactant proteins such as C-reactive protein (CRP), serum amyloid P, serum amyloid A, complement factors, fibrinogen, prothrombin, anti-hemophilic factor (AHF), von Willebrand factor, mannan-binding lectin, plasminogen, alpha 2-macroglobulin, ferritin, hepcidin, ceruloplasmin, haptoglobin, alpha-1-acid glycoprotein (AGP), alpha 1-antitrypsin, alpha 1-antichymotrypsin, and plasminogen activator inhibitor I.

There are standard laboratory tests to detect or diagnose hemo-concentration. Exemplary clinical tests, include, but are not limited to, sedimentation values, a decline in or a low value for StO₂ (tissue oxygenation), measurements showing elevated fibrinogen, elevated RBC count, elevated hematocrit (any value above normal), measurement of RBC aggregation (showing increased aggregation) or RBC sedimentation rate (elevated, anything above the normal range)

Hemo-concentration also can occur in subjects with impaired circulation, such as impaired microcirculation. The microcirculation encompasses vessels that are typically less than 150 μm in diameter, for example, arterioles, capillaries, and venules. For example, the microcirculation includes those arterial vessels that respond to increasing pressure by a myogenic reduction in lumen diameter, as well as the capillaries and venules. Functions of the microcirculation include optimizing nutrient and oxygen supply within tissues in response to variations in demand and avoiding large fluctuations in hydrostatic pressure at the level of the capillaries thereby causing disturbances in capillary exchange. Additionally, it is at the level of the microcirculation that a substantial proportion of the drop in hydrostatic pressure occurs. Thus, the microcirculation is extremely important in determining the overall peripheral resistance, i.e., the resistance of the arteries to blood flow in the systemic circulation. It also is the site where the earliest manifestations of cardiovascular disease, in particular, inflammatory processes, occur.

### C. TREATMENT OF THE SIDE EFFECTS OF DIURESIS, DEHYDRATION AND/OR HEMO-CONCENTRATION BY ADMINISTRATION OF A POLYOXYETHYLENE/POLYOXYPROPYLENE COPOLYMER

Administration of a polyoxyethylene/polyoxypropylene copolymer treats the unwanted side effects and consequences of dehydration and diuresis, e.g., diuretic-induced diuresis. Administration of a polyoxyethylene/polyoxypropylene copolymer to a subject with ameliorates hemo-concentration and microvascular hemodynamic alterations due to dehydration or diuresis can be indicated by a high hematocrit, increased concentration of an acute phase reactant, such as fibrinogen, an elevated erythrocyte sedimentation rate and combinations thereof.

Provided herein are methods for treating side effects or consequences of dehydration or diuresis in a subject. The methods include administering to the subject a therapeutically effective amount of a composition that contains a polyoxyethylene/polyoxypropylene copolymer (poloxamer). Among the poloxamers are any having the chemical formula HO(C₂H₄O)_{a′}—(C₃H₆O)_{b}—(C₂H₄O)ₐH as described throughout the disclosure herein. In particular, among the poloxamers are those where a′ and a are the same or different and each is an integer such that the hydrophile portion represented by (C₂H₄O) constitutes approximately 60% to 90% by weight of the compound; and b is an integer such that the hydrophobe portion represented by (C₃H₆O) has a molecular weight of approximately 1300 to 2300 Daltons (Da), such as approximately or at 1500 to 2100 Da, or about 1700 to 1900 Da, to treat the side effects of diuresis.

Among the poloxamers that can be used are those in which a and a are 5-150, such as 70-105, and b is 15-72 or 15-75. The poloxamer can contain 60-90% Hydrophile, C₂H₄O, I constitutes 80% or 81% of the compound and the hydrophobe is present such that the molecular weight is about or is 1800-1840 Da, such as 1800. The polyoxyethylene/polyoxypropylene copolymer, P188 also includes compounds in which the a and a′ are each 80, b is 27, the hydrophile constitutes approximately 80% (or 80-81%) by weight of the compound, and the molecular weight of the compound is about or is 1750 Da. Others are described above and in the sections that follow.

In some methods, the poloxamer has reduced impurities so that the polydispersity value is less than or equal to approximately 1.07. In some methods, the poloxamer is purified to reduce low molecular weight (LMW) substances. In other methods, the poloxamer has the chemical formula HO(CH₂CH₂O)_{a′}—[CH(CH₃)CH₂O]_{b}—(CH₂CH₂O)ₐH, wherein the molecular weight of the hydrophobe portion [CH(CH₃)CH₂O] is approximately 1700 to 1790 Da, such as about 1750 Da, and the total molecular weight of the poloxamer compound is approximately 8400 to 8800 Da.

The provided methods include administering to the subject a therapeutically effective amount of a composition that contains the polyoxyethylene/polyoxypropylene copolymer (poloxamer) having the chemical formula HO(C₂H₄O)_{a′}—(C₃H₆O)_{b}—(C₂H₄O)ₐH, as described herein and/or known to those of skill in the art, to treat the side effects of diuresis; and administering a therapeutically effective amount of a diuretic. The therapeutically poloxamer can be administered to the subject prior to, concomitant with, or after administration of a diuretic or other treatment, or any combination thereof. The amount and duration of poloxamer administration is sufficient to maintain a target blood concentration that effect treatment. Target blood concentrations can depend upon the particular poloxamer, the subject to whom it is administered, the condition treated, underlying conditions and the severity of the hemo-concentration. Dosages are described herein and also can be determined empirically by the skilled artisan. Generally, the target dosage is one that achieves a circulating concentration of at least 0.05 mg/ml, typically at least 0.5 mg/ml, and generally range of 0.5 mg/ml-1.5 mg/ml. In the methods provided herein, the therapeutically effective amount of poloxamer is an amount that results in a concentration of poloxamer in the circulation of the subject of from about or at 0.2 mg/mL to about or at 4.0 mg/mL, for example, about 0.5 mg/mL-1.5 mg/mL or at least 0.5 mg/ml, at a desired time point, typically steady-state, after administration of the poloxamer. Other ranges are contemplated as well, such as 0.05-10 mg/ml, 0.5-10 mg/ml, and others described herein.

Dosages for other treatments and therapeutic, that are concomitantly administered or administered prior depend upon the therapeutic and condition treated and the regimen. For example, dosages for diuretics, are typically the recommended doses for such diuretics, for example, such as dosages described in standard manuals, including the Physician's Desk Reference and Remington's Pharmaceutical Sciences (Mack Publishing Co., Easton, Pa.). As described, the provided methods include administration of the poloxamer where diuretic therapy results in hemo-concentration and/or microvascular hemodynamic alterations. In some methods, the poloxamer mitigates a side-effect of administering a diuretic, including hemo-concentration of at least one blood plasma protein, blood cells, or combinations thereof. In some examples, the plasma protein is fibrinogen. In some examples, the blood cells are erythrocytes, i.e., red blood cells.

In some methods provided herein, the polyoxyethylene/polyoxypropylene copolymer poloxamer can be administered to treat, prevent or reduce the risk of the complications of hemo-concentration. The poloxamer can be administered in combination with therapy, such as diuretic therapy, for an underlying condition. In other embodiments of the methods, the polyoxyethylene/polyoxypropylene copolymer is administered after the dehydration/diuresis or hemo-concentration is detected or symptoms occur.

In the provided methods, administration of the poloxamer can be repeated, for example, a second, third, fourth time, or more. For example, the method can be repeated until administration of the poloxamer is sufficient to result in a concentration of the poloxamer in the circulation of the subject of from about 0.05 mg/mL to about 10 mg/mL, about 0.05 mg/mL to about 4.0 mg/mL, or about 0.2 mg/mL to about 2.0 mg/mL. In methods where the poloxamer is administered in combination with diuretic treatment, administration of the diuretic can be repeated, for example, a second, third, fourth time, or more.

The following sections describe poloxamers, such as poloxamer 188, and compositions thereof, for use in treating the side effects or consequences of diuresis, including in the treatment of side effects or consequences associated with diuretic-induced diuresis. Exemplary dosage regimes and methods are described.

1. Poloxamers for Preventing and Treating Complications from Hemo-Concentration

Provided herein are methods and uses of a poloxamer for treating complications/side effects of hemo-concentration, particularly that resulting from diuresis and dehydration. Poloxamers include, but are not limited to, a poloxamer 188 (P188), such as a purified P188 (e.g., LCMF), for treating or ameliorating the side effects of diuresis. The methods provided herein for treating the side effects of diuresis include administering a treatment that includes a therapeutically effective amount of a poloxamer to a human or animal subject.

Certain polyoxyethylene/polyoxypropylene copolymers, including P188, have beneficial biological effects on several disorders when administered to a human or animal. These activities have been described in U.S. Pat. Nos. 4,801,452; 4,837,014; 4,873,083; 4,879,109; 4,897,263; 4,937,070; 4,997,644; 5,017,370; 5,028,599; 5,030,448; 5,032,394; 5,039,520; 5,041,288; 5,047,236; 5,064,643; 5,071,649; 5,078,995; 5,080,894; 5,089,260; RE 36,665 (Reissue of U.S. Pat. No. 5,523,492); U.S. Pat. Nos. 5,605,687; 5,696,298; 6,359,014; 6,747,064; 8,372,387; 8,580,245; U.S. Patent Publication Nos. 2011/0044935, 2011/0212047, and 2013/0177524; International Application Nos. PCT/US2005/034790, PCT/US2005/037157 and PCT/US2006/006862; and U.S. Provisional Patent Application No. 60/995,046. Among the activities of poloxamers, such as P188, is their ability to incorporate into cellular membranes, and thereby repair damaged cell membranes.

Poloxamers for use in the methods provided herein include POP/POE block copolymers having the following formula:

HO(C₂H₄O)_{a′}—(C₃H₆O)_{b}—(C₂H₄O)ₐH

wherein “a” and “a” can be the same or different and each is an integer such that the hydrophile portion represented by (C₂H₄O) constitutes approximately 60% to 90%, such as 70% to 90%, by weight of the compound; and “b” is an integer such that the hydrophobe represented by (C₃H₆O) has a molecular weight of approximately 950 to 4000 Da, such as 1200 to 3500 Da, for example, 1300 to 2300 Da. For example, the hydrophobe has a molecular weight of 1200 to 2300 Da, such as generally 1500 to 2100 Da, for example, 1700 to 1900 Da. The average molecular weight of the copolymer is 3000 to 23,000 Da, for example, 5000 to 15,000 Da, such as 5000 to 9000 Da. In some examples, b is an integer of from about 20 to about 40, such or any of the numbers in between. In some examples, b is or is about 15 to about 50, such as about 20 to about 40, or about 25 to about 35, for example, 20, 21, 22, 23, 24, 25, 26, 27, 28, 29, 30, 31, 32, 33, 34, 35, 36, 37, 38, 39, or 40. In some examples, a and a′ are each an integer of about 20 to about 230 or any of the numbers in between, for example, about 40 to about 200, about 50 to about 150, about 60 to about 100, or about 70 to about 90. One of skill in the art will appreciate that these values are average values. The values for a, a′ and b represent an average, where the polymeric molecules are a distribution or population of molecules. The actual values of a, a′ and b within the population constitute a range of values.

Poloxamers for use in the methods herein, including P188, are available from commercial sources. Alternatively, poloxamers can be synthesized using standard polymer synthesis techniques including any described in the US Patents listed above. They also can be synthesized as described herein in the Examples.

Generally, poloxamers are formed by ethylene oxide-propylene oxide condensation using standard techniques know to those of skill in the art (see, e.g., U.S. Pat. Nos. RE 36,665; RE 37,285; RE 38,558; 6,747,064; 6,761,824; and 6,977,045; see also, Reeve, L. E., “The Poloxamers: Their Chemistry and Medical Applications,” in Handbook of Biodegradable Polymers, Domb, A. J. et al. (eds.), Hardwood Academic Publishers, 1997). Poloxamers can be synthesized by sequential addition of POP and POE monomers in the presence of an alkaline catalyst, such as sodium or potassium hydroxide (see, e.g., Schmolka (1977) J. Am. Oil Chem. Soc. 54:110-116). The reaction is initiated by polymerization of the POP block followed by the growth of POE chains at both ends of the POP block. Methods of synthesizing polymers also are described in U.S. Pat. No. 5,696,298.

As noted above, poloxamers nomenclature relates to the composition of the various polymer members. The first two digits of a poloxamer number, multiplied by 100, gives the approximate molecular weight of the hydrophobe, i.e., polyoxypropylene, content. The last digit, multiplied by 10, gives the approximate weight percent of the hydrophile, i.e., polyoxyethylene, content of the copolymer. For example, poloxamer 407 describes a polymer containing a polyoxypropylene hydrophobe of about 4000 Da with the polyoxyethylene hydrophile comprising about 70% of the total molecular weight. Poloxamer 188 (P188) has a hydrophobe with a molecular weight of about 1800 Da and has a hydrophile that is about 80% of the total molecular weight of the copolymer.

Exemplary poloxamers for use in the methods herein include, but are not limited to, poloxamer 136, poloxamer 137, poloxamer 138, poloxamer 139, poloxamer 146, poloxamer 147, poloxamer 148, poloxamer 149, poloxamer 156, poloxamer 157, poloxamer 158, poloxamer 159, poloxamer 166, poloxamer 167, poloxamer 168, poloxamer 169, poloxamer 176, poloxamer 177, poloxamer 178, poloxamer 179, poloxamer 186, poloxamer 187, poloxamer 188, poloxamer 189, poloxamer 196, poloxamer 197, poloxamer 198, poloxamer 199, poloxamer 206, poloxamer 207, poloxamer 208, poloxamer 209, poloxamer 216, poloxamer 217, poloxamer 218, poloxamer 219, poloxamer 226, poloxamer 227, poloxamer 228, poloxamer 229, poloxamer 236, poloxamer 237, poloxamer 238, poloxamer 239 and variants thereof.

Poloxamers are sold and frequently referred to under trade names and sold under trademarks, including, but not limited to, ADEKA NOL, Synperonic™, Pluronic® and Lutrol®. Exemplary of such poloxamers, but not limited to, are poloxamer 188 (P188; sold under the trademarks Pluronic® F-68, Kolliphor® P 188, RheothRX Rx and Flocor™; 80% POE), poloxamer 407 (P407; sold under the trademarks Lutrol F 127, Kolliphor® P 407 and Pluronic® F-127; 70% POE), poloxamer 237 (P237; sold under the trademarks Pluronic® F87 and Kolliphor® P 237; 70% POE.

Poloxamers for use in the methods herein, including P188, also include preparations of a poloxamer that are further purified to remove particular components, generally LMW and HMW components. As noted above, unlike discrete molecules that have a single, defined chemical structure, poloxamers can be molecularly diverse Specific poloxamers are composed of multiple chemical entities that have the POE-POP-POE structural motif, but vary in the number of repeating POE and POP units. The molecular diversity is the product of the processes by which poloxamers are synthesized. The result is material that is non-uniform (i.e., material that is polydisperse). Adding to this polydispersity is a variety of other substances that can form as a result of side reactions occurring during synthesis of the intended poloxamer compound. These other substances can be present and found within the overall poloxamer distribution. In some examples, the poloxamer has reduced impurities. For example, in particular examples, the copolymer has been purified, for example, to remove or reduce the amount of certain low molecular weight impurities and other components, so that the polydispersity value is less than approximately 1.07. Methods for purifying poloxamers are known (see, e.g., U.S. Pat. No. 5,567,859). Methods, such as supercritical fluid extraction methods, are described herein.

In some embodiments, chemically modified forms of one or more poloxamers are employed in the compositions, methods and uses provided herein. Chemical modifications of poloxamers include, but are not limited to, radiolabelling, acetylating, biotinylation, addition of a fluorophore, and other chemical modifications known to those of skill in the art.

2. Poloxamer 188

Exemplary of a poloxamer in the compositions methods and uses provided herein is poloxamer 188 (P188) and purified preparations thereof. A P188 copolymer has the following chemical formula:

HO(CH₂CH₂O)_{a′}—[CH(CH₃)CH₂O]_{b}—(CH₂CH₂O)ₐH

wherein the hydrophobe represented by [CH(CH₃)CH₂O] has a molecular weight of approximately 1700 to 1800 Da, such as 1750 Da, and an average molecular weight of 7680 to 9510 Da, such as generally approximately 8400 to 8800 Da. The polyoxyethylene-polyoxypropylene-polyoxyethylene weight ratio of P188 is approximately 4:2:4. P188 has a weight percent of oxyethylene of 81.8±1.9%, and an unsaturation level of 0.026±0.008 mEq/g.

P188 is a polyoxyethylene/polyoxypropylene linear copolymer that is a surface-active agent, or surfactant. As a surface active agent, P188 binds to hydrophobic areas developed on injured cells and denatured proteins, thereby restoring hydration lattices. Non-purified P188 is commercially available and sold under various trademarks as noted above. These include P188, for example, sold under the trademarks Pluronic® F-68 (BASF, Florham Park, N.J.) and RheothRX Rx® (developed by Glaxo Wellcome, Inc.). Because of the synthesis procedure there can be variation in the rates of polymerization during the steps of building the POP core and POE terminal ends. Thus, forms of P188 contain a bell-shaped distribution of polymer species, which vary primarily in overall chain length. In addition, various low molecular weight (LMW) components formed by incomplete polymerization (e.g., glycols and truncated polymers), and high molecular weight (HMW) components (e.g., dimerized polymers) can be present. Characterization of P188 by gel permeation chromatography (GPC) identifies a main peak of P188 with “shoulder” peaks representing the unintended LMW and HMW components (see, Emanuele and Balasubramanian (2014) Drugs R D 14:73-83). Studies of the therapeutic potential of P188 led to the discontinuance of RheothRX Rx® P188 for therapeutic applications in part due to an acute renal dysfunction observed during clinical trial evaluation. These effects are due to the presence of various low molecular weight (LMW) substances that formed during the synthesis (Emanuele and Balasubramanian (2014) Drugs R D 14:73-83). Hence, in purified P188 these LMW components are removed.

In the methods and uses provided herein, while any suitable poloxamer preparation can be used, the P188 typically is purified to remove the LMW components. A purified P188 has a polydispersity value of the polyoxypropylene/polyoxyethylene block copolymer that is less than or equal to approximately 1.07, such as less than or equal to approximately 1.05, and generally less than or equal to approximately 1.03. The purified P188 typically has reduced LMW components. In addition, it can be purified to removed the HMW components. A purified P188, an LCMF P188, has a reduction in HMW components.

Exemplary of a purified P188 is a P188 purified to reduce or remove LMW components (e.g., as described in U.S. Pat. No. 5,696,298 or known under the trademark Flocor™), and a longer circulating material free (LCMF) P188 as described herein (see, also U.S. provisional application Ser. No. 62/021,697) A P188 can be purified using various extraction processes known in the art, for example, any extraction process that can remove components, such as LMW and/or HMW components, from the poloxamer. Such methods include any methods provided herein, and but are not limited to, high pressure extraction or supercritical fluid extraction (SFE) methods.

3. Longer Circulating Material Free (LCMF) Poloxamer

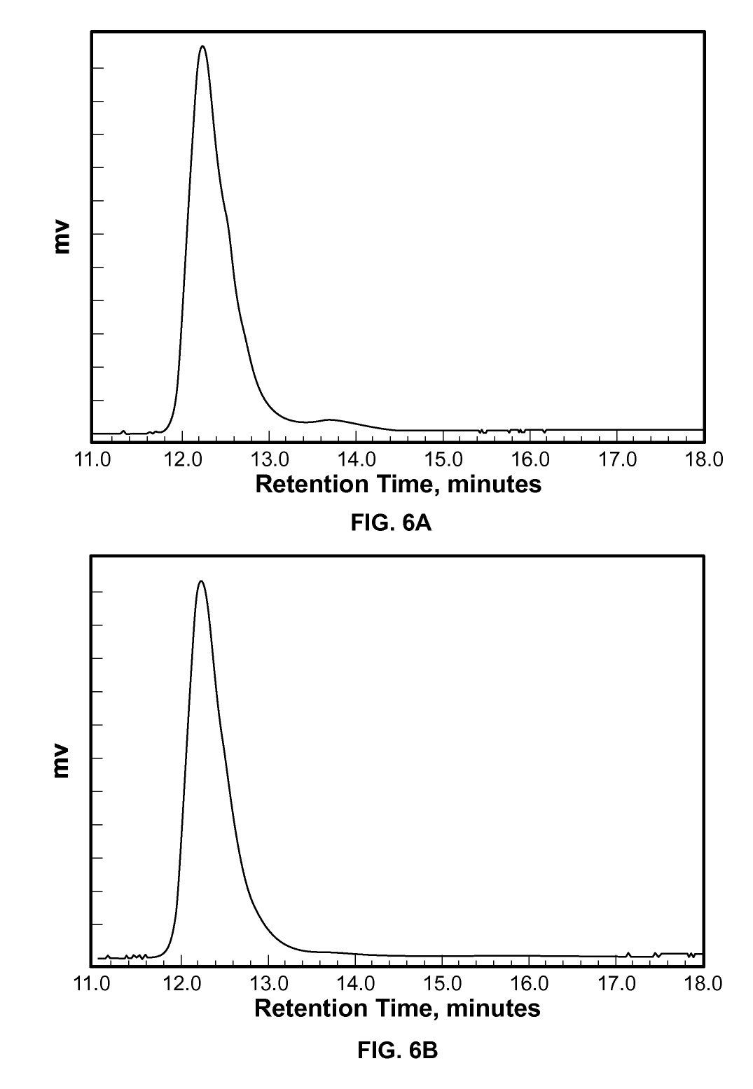

As described herein, and in U.S. provisional application. Ser. No. 62/021,697 (see, also International PCT application No. PCT/US14/45627), a component in P188 has been identified that is or gives rise to a circulating material in the plasma or blood with a longer circulating half-life and with longer retention time than the main or predominant poloxamer peak species. For example, in non-clinical and clinical studies, analysis of plasma obtained following intravenous administration of purified P188 by high performance liquid chromatography-gel permeation chromatography (HPLC-GPC) shows two distinct peaks in the circulation (see, e.g., Grindel et al. (2002) Journal of Pharmaceutical Sciences, 90:1936-1947 or Grindel et al. (2002) Biopharmaceutics & Drug Disposition, 23:87-103). There is a main peak with an average molecular weight of about 8600 Da and a half-life of about 7 hours and a smaller peak with an average molecular weight of about 16,000 Da and a half-life or approximately 70 hours. Thus, the two peaks exhibit distinctly different pharmacokinetic profiles, with the higher molecular weight peak exhibiting a distinctly longer plasma residence time with slower clearance from the circulation (see FIG. 6A and FIG. 6B). Similar observations were reported in rats and dogs.

Since the rheologic, cytoprotective, anti-adhesive and anti-thrombotic effects of P188 are observed with the predominant or main copolymers of the distribution, which are approximately 8400 to 9400 Da and have a half-life of about 7 hours, the presence of other components that exhibit a long circulating half-life can result in unwanted side effects. For example, among the desired activities of P188 is its rheologic effect to reduce blood viscosity and inhibit red blood cell (RBC) aggregation, which accounts for its ability to improve blood flow in damaged tissues. In contrast, other poloxamers, such as P338 (sold under the trademark Pluronic® F-108) and P278 (Pluronic® F98), increase blood viscosity, can increase RBC aggregation (Armstrong et al. (2001) Biorheology 38:239-247). This result correlates with an increase in the molecular weight of the POP component, as opposed to the percentage of POE content. Intravenously injected emulsions of P238 or P338, which are higher molecular weight poloxamer members with 80% POE, are described as remaining in the blood for relatively long periods (Moghimi, S. M. (1998) “Recent Developments and Limitations of Poloxamine-Coated Long-Circulating Particles in Experimental Drug Delivery,” in Gregoriadis and McCormack (eds) Targeting of Drugs 6: Strategies For Stealth Therapeutic Systems, pp. 263-274, New York: Plenum Press). For example, the average molecular weight of P338 is 12,700 to 17,600 Da. Therefore, since the HMW component observed in P188 is a block polymer with an estimated molecular weight of greater than 13,000 Da, such as about 16,000 Da, its presence as a long circulating material should have a negative therapeutic effect by its opposing action compared to the main or predominant copolymer in the distribution. Hence, the LCMF P188 exhibits improved properties.

Thus, exemplary of a purified poloxamer is a longer circulating material free (LCMF) poloxamer, including an LCMF P188. This LCMF is a poloxamer that is a purified P188 that has a polydispersity value of less than 1.07; no more than 1.5% of low molecular weight (LMW) components less than 4500 daltons; no more than 1.5% of high molecular weight components greater than 13,000 daltons; a half-life of all components in the distribution of the copolymer, when administered to a subject, that is no more than 5.0-fold longer half-life in the blood or plasma than the half-life of the main peak in the distribution of the copolymer. The LCMF P188 polymers have formulae as described herein for P188, including the following chemical formula:

HO(CH₂CH₂O)_{a′}—[CH(CH₃)CH₂O]_{b}—(CH₂CH₂O)ₐH

wherein “a′” and “a” can be the same or different and each is an integer such that the hydrophile portion represented by (CH₂CH₂O) (i.e., the polyoxyethylene portion of the copolymer) constitutes approximately 60% to 90%, such as approximately 80% or 81% of the copolymer; “b” is an integer such that the hydrophobe represented by [CH(CH₃)CH₂O] (i.e., the polyoxypropylene portion of the copolymer) has a molecular weight of approximately 1300 to 2300 Da, such as approximately 1750 Da; and the average total molecular weight of the compound is approximately 7680 to 9510 Da (main peak), such as generally 8400 to 8800 Da, for example, about or at 8000 Da or 8400 Da. The copolymer has been purified to remove impurities, including low molecular weight impurities and other impurities, so that the polydispersity value is less than 1.07. The product is prepared such that HMW impurities also are not present.

Studies have demonstrated that the main peak of a purified P188 preparation, when administered to a human subject, has a half-life (t_{1/2}) in plasma of about 7 hours (see, Grindel et al. (2002) Journal of Pharmaceutical Sciences, 90:1936-1947 and Grindel et al. (2002) Biopharmaceutics & Drug Disposition, 23:87-103) In contrast, higher molecular weight components, such as those having an average molecular weight of about 16,000 Da, exhibit about a 10-fold or more increase in half-life with a t_{1/2} of approximately 70 hours.

In contrast to the purified P188 characterized in the studies of Grindel et al., ((see Grindel et al. (2002) Journal of Pharmaceutical Sciences, 90:1936-1947 or Grindel et al. (2002) Biopharmaceutics & Drug Disposition, 23:87-103)), the purified poloxamer, designated LCMF 188, is one in which all components of the polymeric distribution, when administered to a subject, clear from the circulation at approximately the same rate. A preparation of LCMF poloxamer contains a substantially polydisperse composition of less than 1.07, and generally less than 1.05 or 1.03, where the half-life in the blood or plasma of any component in the distribution of the copolymer, when administered to a subject, is no more than 5.0-fold longer than the half-life of the main peak in the distribution of the copolymer, and generally no more than 4.0-fold, 3.0-fold, 2.0-fold, or 1.5-fold longer. Typically, the LCMF does not contain any component that exhibits a half-life in the blood or plasma, when administered to a subject, that is substantially more than or is more than the main peak in the distribution of the copolymer.

In some examples, the half-life in the blood or plasma of all components in the LCMF poloxamer, when administered to a human subject, is such that no component has a half-life that is more than 30 hours, and generally is no more than 25 hours, 20 hours, 15 hours, 10 hours, 9 hours, 8 hours, or 7 hours.

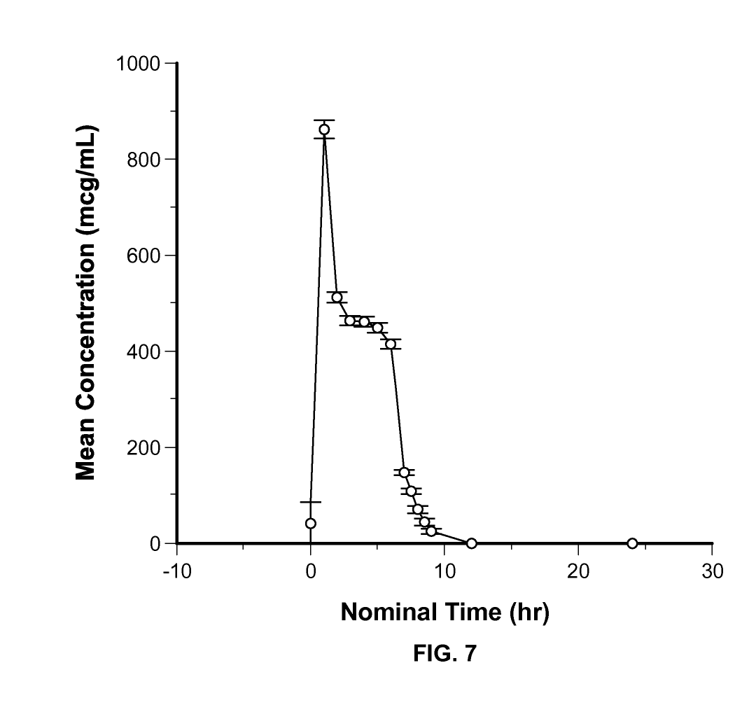

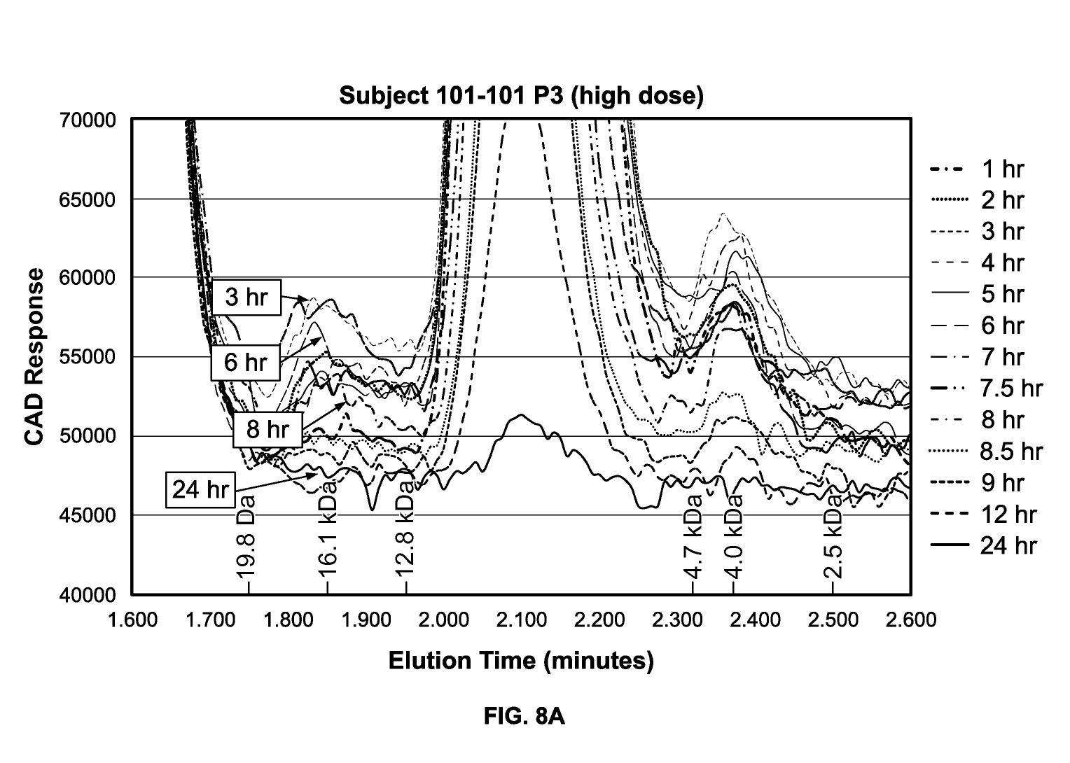

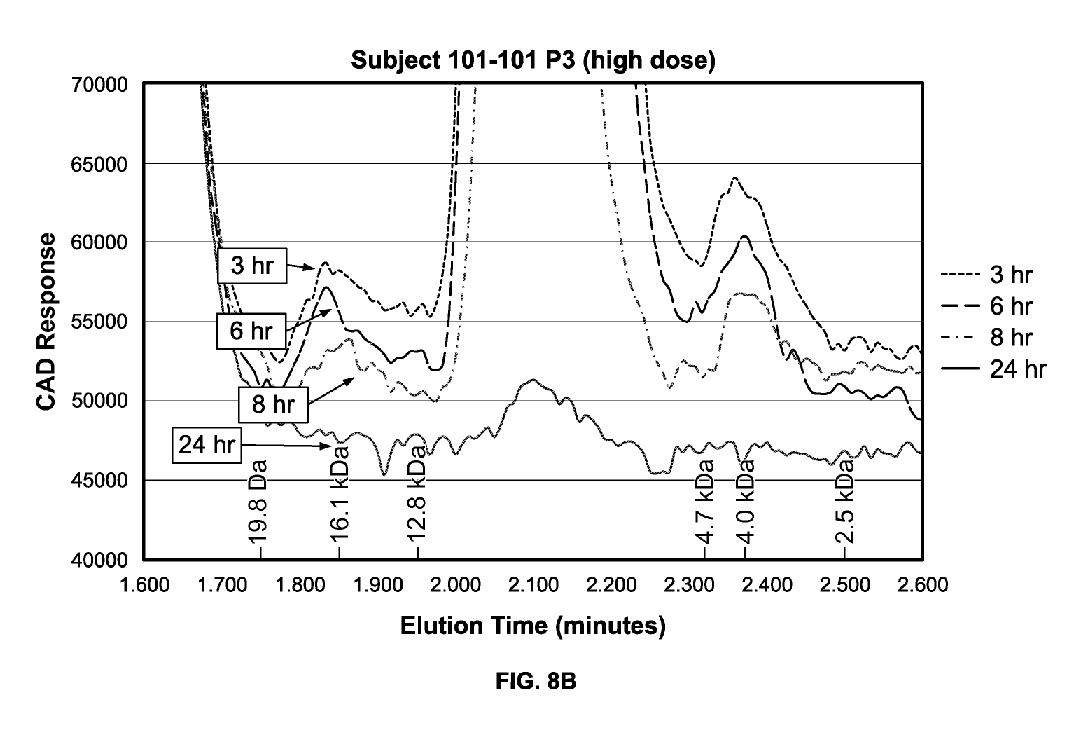

Without wishing to be bound by theory, the higher molecular weight components greater than 13,000 Da could account for the longer circulating half-life material. For example, as described above, studies have shown that higher molecular weight polymers (e.g., P238 or P338), including those with an average molecular weight greater than 13,000 Da, have a longer retention time in the plasma than other polymers (Moghimi, S. M. (1998) “Recent Developments and Limitations of Poloxamine-Coated Long-Circulating Particles in Experimental Drug Delivery,” in Gregoriadis and McCormack (eds) Targeting of Drugs 6: Strategies For Stealth Therapeutic Systems, pp. 263-274, New York: Plenum Press). In some examples, an LCMF preparation provided herein includes HMW components in the distribution that exhibit different properties that do not result in a longer circulating species. For example, HMW impurities greater than 13,000 Da in an LCMF preparation, which generally is no more than 1.5% by weight of the total components, do not, when the LCMF preparation is administered to a subject, result in a circulating half-life that is substantially more than or is more than the main peak in the distribution (see e.g., FIG. 7 and FIGS. 8A and 8B). For example, the HMW impurities greater than 13,000 Da in an LCMF preparation, which generally is no more than 1.5% by weight of the total components, do not, when the LCMF preparation is administered to a subject, result in a circulating half-life that is more than 5.0-fold longer than the half-life of the main peak in the distribution, and generally no more than 4.0-fold, 3.0-fold, 2.0-fold, or 1.5-fold longer.

In some examples, the HMW components are removed or reduced in an LCMF preparation compared to other existing purified P188 preparations. Any method for such purification is contemplated, including the exemplified method of preparation that results in a product that does not have this component. For example, an LCMF poloxamer provided herein includes P188 poloxamers in which there are no more than 1.3% high molecular weight components greater than 13,000 Da, such as no more than 1.2%, 1.1%, 1.0%, or less. In particular examples provided herein, an LCMF poloxamer provided herein includes P188 poloxamers in which there are less than 1.0% by weight high molecular weight components greater than 13,000 Da, and generally less than 0.9%, 0.8%, 0.7%, 0.6%, 0.5%, or less.

3. Supercritical Fluid Extraction Methods to Purify Poloxamers

Any method known to a skilled artisan can be used to purify a poloxamer. In particular, supercritical methods can be employed. Supercritical extraction permits control of the solvent power by manipulation of temperature, pressure and the presence of a co-solvent modifier. It is found that carbon dioxide is not a particularly efficient extraction solvent of poloxamers, such as P188, but that the presence of a polar co-solvent, such as an alkanol, as a modifier can increase the solubilizing efficiency of the extraction solvent. In particular, extraction methods described are performed in the presence of a polar co-solvent, such as an alkanol, whose concentration is increased in a gradient fashion (e.g., a step-wise gradient or a continuously escalating gradient) as the extraction process progresses. It is found that by employing an alkanol co-solvent whose concentration is increased in this manner, the removal of impurities can be increased, and to a much greater extent than when carbon dioxide is used alone. For example, an extraction method that uses carbon dioxide alone is not capable of removing the unwanted components, such as the LMW components or HMW components described herein, to the same degree as that achieved by the provided method.

In the methods of purifying a poloxamer using supercritical fluid extraction methods, the LMW components or impurities of a poloxamer distribution can be selectively removed with a lower alkanol (e.g., methanol) concentration and higher pressure than other HMW components in the distribution. As described further below, by increasing the solubilizing power of the extraction solvent, for example by carefully controlling the pressure and concentration of polar solvent, such as an alkanol (e.g., methanol), it also is possible to remove other impurities. In particular, a method is provided employing a gradient of higher concentrations of an alkanol (e.g., methanol), alone or in conjunction with a decrease in the pressure, that results in the removal of components (e.g., HMW components) in a poloxamer distribution that, when administered to a subject, is or gives rise to a longer circulating material in the plasma.

There is, however, a tradeoff with respect to the yield of poloxamer. Generally, as the concentration of the alkanol (e.g., methanol) co-solvent increases, the solvating power of the extraction solvent is increased so that more compounds are solubilized and the degree of extraction increases. By increasing the concentration of extraction solvent in a gradient fashion, the reduction of poloxamer yield is minimized, while the purity of the final product is maximized. Typically, the methods achieve a yield such that the amount of the extracted or purified polymer obtained by the method is at least 55%, 60%, 70%, 75%, 80%, 85%, 90%, or more of the starting amount of the poloxamer prior to performance of the method. The resulting poloxamers, however, exhibit a substantially greater purity with a higher percentage of main peak in the distribution than the starting material, and without impurities that exhibit toxic side effects or that can result in a longer circulating material in the plasma when administered.

The methods can be performed on any poloxamer in which it is desired to increase the purity, for example by decreasing or reducing components that are undesired in the distribution of a polymer. It is within the level of a skilled artisan to choose a particular poloxamer for use in the methods. An undesired component is any that is or gives rise to a material that is toxic or that has a biological activity that is opposing or counter to the desired activity. For example, the poloxamer can be one in which it is desired to reduce or remove LMW components in the poloxamer, for example, any LMW components that result in acute renal side effects, such as elevated creatine, when administered. The poloxamer also can be one that contains any component, such as an HMW component, that, when administered, is or gives rise to a material that has a half-life in the blood that is different (e.g., longer) from the half-life of the main peak in the distribution of the polymer. Such components can increase blood viscosity and red blood cell aggregation, and hence are undesired.

For example, the extraction methods provided herein can be employed to purify a P188 preparation, where the P188 preparation has the following chemical formula:

HO(CH₂CH₂O)_{a′}—[CH(CH₃)CH₂O]_{b}—(CH₂CH₂O)ₐH

wherein the hydrophobe represented by [CH(CH₃)CH₂O] has a molecular weight of approximately 1700 to 1800 Da, such as 1750 Da, and an average molecular weight of 7680 to 9510 Da, such as generally approximately 8400 to 8800 Da. The polyoxyethylene-polyoxypropylene-polyoxyethylene weight ratio of P188 is approximately 4:2:4. P188 has a weight percent of oxyethylene of 81.8±1.9%, and an unsaturation level of 0.026±0.008 mEq/g. P188 preparations for use in the methods herein include commercially available preparations. These include, but are not limited to, poloxamers sold under the trademarks Pluronic® F-68 (BASF, Florham Park, N.J.) and RheothRx® (developed by Glaxo Wellcome Inc.).

The LCMF poloxamer provided herein can be prepared by methods as described herein below. For example, an LCMF poloxamer provided herein is made by a method that includes:

a) introducing a poloxamer solution into an extractor vessel, wherein the poloxamer is dissolved in a first alkanol to form a solution;

b) contacting the poloxamer solution with an extraction solvent that includes a second alkanol and supercritical carbon dioxide under a temperature and pressure to maintain the supercritical carbon dioxide for a first defined period, wherein:
the temperature is above the critical temperature of carbon dioxide but is no more than 40° C.;
the pressure is 220 bars to 280 bars; and
the alkanol is provided at an alkanol concentration that is 7% to 8% by weight of the total extraction solvent; and

c) increasing the concentration of the second alkanol in step b) in the extraction solvent a plurality of times in gradient steps over time of the extraction method, wherein:

each plurality of times occurs for a further defined period; and

in each successive step, the alkanol concentration is increased 1% to 2% compared to the previous concentration of the second alkanol; and

d) removing the extraction solvent from the extractor vessel to thereby remove the extracted material from the raffinate poloxamer preparation.

a. Process for Extraction

The supercritical fluid extraction process is essentially a solvent extraction process using a supercritical fluid as the solvent. With supercritical fluid extraction, multi-component mixtures can be separated by exploiting both the differences in component volatilities and the differences in the specific interactions between the component mixture and supercritical fluid solvent (solvent extraction). In the supercritical region of the phase diagram, a compressible fluid such as carbon dioxide exhibits liquid-like density and a much increased solvent capacity that is pressure dependent.

The supercritical fluid exhibits a number of highly advantageous characteristics making it a superior solvent. For example, the tunable solvent power of a supercritical fluid changes rapidly around critical conditions within a certain range. The solvent power of the supercritical fluid, and thus the nature of the component that can be selectively removed during extraction, can be fine-tuned by varying the temperature and pressure of the supercritical fluid solvent.

Another beneficial property of various supercritical fluids is the difference in their critical temperatures and pressures. Each supercritical fluid has a range of solvent power. The tunable solvent power range can be selected by choosing an appropriate supercritical fluid.

In addition to the unique solubility characteristics, supercritical fluids exhibit certain physicochemical properties making them useful. For example, supercritical fluids exhibit liquid-like density and possess gas-like transport properties, such as diffusivity and viscosity. These characteristics also change rapidly around the critical region. Supercritical fluids also have zero surface tension. Since most of the useful supercritical fluids have boiling points around or below ambient temperature, the solvent removal step after purification is simple, energy efficient and does not leave any residual solvents.

In addition, use of solid matrices during extraction provides an additional dimension for a fractionation parameter. Use of a suitable solid matrix provides solvent-matrix and solute-matrix interactions in addition to solute-solvent interaction to enhance the fractionation resolution.

Desirable transport properties of supercritical fluids render the process easily scalable for manufacturing. Heat transfer and mass transfer characteristics do not significantly change upon process scale up with supercritical fluid extraction processes. Since the extraction process conditions, such as pressure, temperature, and flow rate, can be precisely controlled, the purification process is reproducible in addition to highly tunable.

In such a method, the extraction solvent can contain a supercritical liquid (e.g., supercritical carbon dioxide), as well as another co-solvent modifier, generally an alkanol, that is increased over time in the extraction. As described above, the presence of the co-solvent modifier can improve the solubility of solutes, such as higher molecular weight or more non-polar solutes, and thereby increase their extraction in the method.

For example, the method provided herein can include: a) providing or introducing a poloxamer (e.g., P188) solution into an extractor vessel, wherein the poloxamer solution is prepared by dissolving the poloxamer in a first alkanol to form the solution; b) admixing an extraction solvent containing a second alkanol and a supercritical liquid, under high pressure and high temperature sufficient to create supercritical liquid conditions, with the poloxamer solution to form an extraction mixture, wherein the concentration of the second alkanol in the extraction solvent is increased over the time of extraction method; and c) removing the extraction solvent from the extractor vessel to thereby remove the impurities (e.g., LMW component or other components) from the poloxamer preparation. The first and second alkanol can be the same or different. In the method, the step of dissolving the poloxamer in the first alkanol can occur prior to charging the solution into an extraction vessel or at the time of charging the solution into an extraction vessel. For example, the poloxamer is dissolved in a separate vessel and then the solution is added to the extraction vessel.

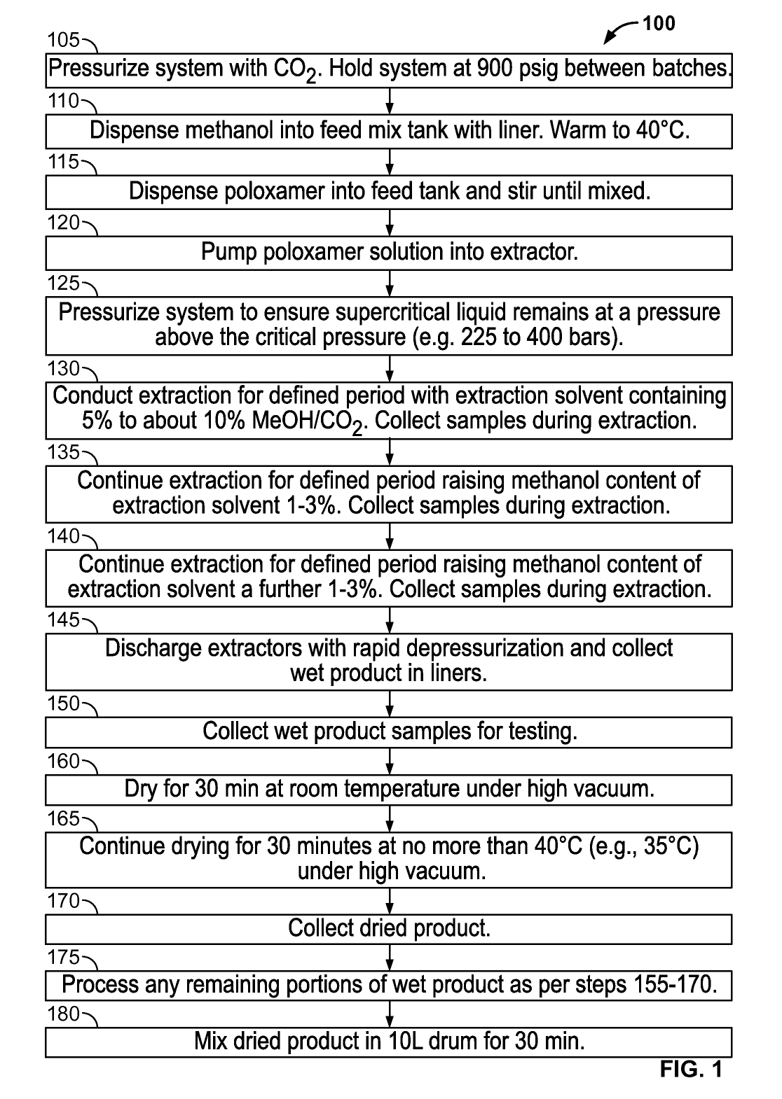

FIG. 1, depicts a process 100 that is useful for removing impurities (e.g., LMW component or other components) from a poloxamer preparation. The extraction system is pressurized, as shown in step 105, typically prior to dispensing a first alkanol into the feed mix tank, as shown in step 110. The system is heated to a temperature suitable for the extraction process. The temperature is typically a temperature that is above the critical temperature of the supercritical liquid (e.g., carbon dioxide). Generally, the temperature is no more than 40° C.

Any suitable alkanol or combination of alkanols can be used in the methods of purifying a poloxamer. Examples of suitable alkanols include, but are not limited to, methanol, ethanol, propanol, butanol, and the like. For example, the method provided herein includes an extraction method as described above, wherein the first and the second alkanol are each independently selected from methanol, ethanol, propanol, butanol, pentanol, and a combination thereof. In some embodiments, the first alkanol is methanol. In certain examples of the method, methanol is selected as the purification solvent and is the second alkanol. A skilled artisan will appreciate that methanol has relatively low toxicity characteristics. Methanol has good solubility for poloxamer 188.

The first alkanol (e.g., methanol) is used to form a poloxamer solution according to step 115 in process 100. A poloxamer, such as a P188 preparation, is dispensed into the feed tank and is stirred until mixed with the first alkanol. The amount of poloxamer that is added to the feed tank is a function of the scalability of the extraction method, the size of the extraction vessel, the degree of purity to achieve, and other factors within the level of a skilled artisan. For example, non-limiting amounts of poloxamer (e.g., P188) per mL of an extraction vessel can be 0.1 kg to 0.5 kg or 0.2 kg to 0.4 kg. In some examples, in methods of extraction using a 3 L extraction vessel, non-limiting amounts of poloxamer (e.g., P188) can be 0.6 kg to 1.2 kg, such as 0.8 kg to 1.0 kg. In another example, in methods of extraction using a 12 L extraction vessel, non-limiting amounts of poloxamer (e.g., P188) can be 1.5 kg to 5 kg, such as 2 kg to 4 kg. In a further example, in methods of extraction using a 50 L extraction vessel, non-limiting amounts of poloxamer (e.g., P188) can be 8 kg to 20 kg, such as 10 kg to 16 kg or 12 kg to 15 kg. Variations in the amounts are contemplated depending on the particular applications, extraction vessel, purity of the starting material, and other considerations within the level of a skilled artisan.

Any suitable ratio of poloxamer and alkanol is contemplated for use in the methods of purifying a poloxamer. The ratio of poloxamer to alkanol, by weight, can be, for example, from about 4:1 to about 1:4, such as from about 3:1 to about 1:3, 2:1 to about 1:2, 1:1 to 4:1 or 1:2 to 1:4. For example, the ratio of poloxamer to alkanol, by weight, can be about 4 to 1, or about 3 to 1, or about 2 to 1, or about 1 to 1, or about 1 to 2, or about 1 to 3, or about 1 to 4. For example, a quantity of poloxamer, such as P188, can be mixed with an equal quantity, by weight, of alkanol (e.g., methanol). A quantity of poloxamer, such as P188, can be mixed with a lesser amount, by weight, of alkanol, such as half the amount, by weight, of alkanol (e.g., methanol). One of skill in the art will appreciate that the appropriate poloxamer to alkanol ratio will depend on poloxamer properties, such as solubility in a given alkanol.

After forming a poloxamer/alkanol mixture, all or part of the mixture is pumped into the extractor as shown in step 120. In such examples, the process of preparing the poloxamer solution is performed in a separate vessel from the extractor. A skilled artisan will appreciate that the poloxamer also can be introduced as a solid into the extractor prior to mixing with the first alkanol. Thus, the process of preparing the poloxamer solution can be made directly in the extractor vessel.

The extractor is then pressurized and the extraction solvent is introduced into the extractor as shown in step 125 of process 100. The extraction solvent contains the supercritical liquid. Examples of supercritical fluids include, but are not limited to, carbon dioxide, methane, ethane, propane, ammonia, freon, water, ethylene, propylene, methanol, ethanol, acetone, and combinations thereof. In some embodiments, the supercritical liquid under pressure is a member selected from carbon dioxide, methane, ethane, propane, ammonia, and freon. In some embodiments, the supercritical liquid under pressure is carbon dioxide (CO₂).

The extraction occurs under high pressure and high temperature to maintain a supercritical liquid condition. Typically, the pressure and temperature are kept constant. At this pressure and temperature, the supercritical liquid (e.g., supercritical carbon dioxide) is provided at a substantially constant flow rate. The flow rate can be varied between 0.5 kg/h and 600 kg/h, such as 1 kg/h to 400 kg/h, 1 kg/h to 250 kg/h, 1 kg/h to 100 kg/h, 1 kg/h to 50 kg/h or 1 kg/h to 20 kg/h, or 1 kg/h to 10 kg/h, 10 kg/h to 400 kg/h, 10 kg/h to 250 kg/h, 10 kg/h to 100 kg/h, 10 kg/h to 50 kg/h, 10 kg/h to 20 kg/h, 20 kg/h to 400 kg/h, 20 kg/h to 250 kg/h, 20 kg/h to 100 kg/h, 20 kg/h to 50 kg/h, 50 kg/h to 400 kg/h, 50 kg/h to 250 kg/h, 50 kg/h to 100 kg/h, 100 kg/h to 400 kg/h, 100 kg/h to 200 kg/h or 200 kg/h to 400 kg/h, each inclusive. For example, the flow rate is 20 kg/h to 100 kg/h, inclusive, such as generally about or 100 kg/h.

Any suitable temperature that maintains the supercritical fluid in the supercritical state can be used to conduct the extraction processes. For example, the critical temperature of carbon dioxide is about 31° C. Thus, the extractor vessel is kept at a temperature greater than 31° C. In some embodiments, the extractor vessel has a temperature of 32° C. to 80° C., and generally 32° C. to 60° C. or 32° C. to 50° C., each inclusive. For example, the temperature can be a temperature that is no more than 35° C., 36° C., 37° C., 38° C., 39° C., 40° C., 41° C., 42° C., 43° C., 44° C., 45° C., 50° C., or 60° C. Generally the temperature is greater than 31° C. but no more than 40° C. One of skill in the art will appreciate that the temperature can be varied, depending in part on the composition of the extraction solvent as well as the solubility of a given poloxamer in the solvents employed in the process.

Any suitable pressure can be used in the methods of the present invention. When supercritical fluid extraction is employed, the system is pressurized at a level to ensure that the supercritical liquid remains at a pressure above the critical pressure. For example, the critical pressure of carbon dioxide is about 74 bars. Thus, the extractor vessel is pressurized to greater than 74 bars. The particular degree of pressure can alter the solubility characteristics of the supercritical liquid; therefore, the particular pressure chosen can affect the yield and degree of extraction of impurities. Typically, the extractor vessel is pressurized in a range of 125 to 500 bar. In some embodiments, the extractor vessel is pressurized in a range of 200 bars to 400 bars, 200 bars to 340 bars, 200 bars to 300 bars, 200 bars to 280 bars, 200 bars to 260 bars, 200 bars to 240 bars, 200 bars to 220 bars, 220 bars to 400 bars, 220 bars to 340 bars, 220 bars to 300 bars, 220 bars to 280 bars, 220 bars to 260 bars, 220 bars to 240 bars, 240 bars to 400 bars, 240 bars to 340 bars, 240 bars to 300 bars, 240 bars to 280 bars, 240 bars to 260 bars, 260 bars to 400 bars, 260 bars to 340 bars, 260 bars to 300 bars, 260 bars to 280 bars, 280 bars to 400 bars, 280 bars to 340 bars, 280 bars to 300 bars, or 300 bars to 340 bars. For example, the extraction vessel can be pressurized at about or at least 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, or 400 bars, but generally no more than 500 bars. The extraction vessel can be pressurized, for example, at 310±15 bars.

Typically, in the methods provided herein, the extraction solvent introduced into the extraction vessel also contains an alkanol. Thus, the extraction solvent includes a second alkanol and a supercritical liquid under high pressure and high temperature. The second alkanol acts as a co-solvent modifier of the supercritical liquid to change the solvent characteristics of the supercritical liquid and improve extractability of the solute in the method. Any suitable alkanol or combination of alkanols, as described above, can be used as the second alkanol in the methods provided herein. As described above, in particular examples, the second alkanol is methanol.

Any suitable combination of the second alkanol and the supercritical liquid, such as any described above, can be used in the extraction solvent in the methods of the present invention. In some embodiments, the extraction solvent includes methanol and carbon dioxide. The second alkanol is typically provided as a percentage (w/w) of the total extraction solvent that is 3% to 20%, and generally 3% to 15%, for example 5% to 12%, 5% to 10%, 5% to 9%, 5% to 8%, 5% to 7%, 7% to 15%, 7% to 12%, 7% to 10%, 7% to 9%, 7% to 8%, 8% to 15%, 8% to 12%, 8% to 10%, 8% to 9%, 9% to 15%, 9% to 12%, 9% to 10%, 10% to 15%, or 10% to 12%, each inclusive. The flow rate (kg/h) of the alkanol is a function of the amount of alkanol introduced into the extractor.

For example, a suitable ratio of alkanol (e.g., methanol) to supercritical liquid (e.g., carbon dioxide) can be selected based on the identity and purity of the poloxamer starting material, or based on other extraction parameters, such as temperature or pressure. For example, the ratio of alkanol (e.g., methanol) to supercritical liquid (e.g., carbon dioxide) can be from about 1:100 to about 20:100. In some embodiments, the ratio of alkanol (e.g., methanol) to supercritical liquid (e.g., carbon dioxide) is from about 1:100 to about 15:100. In some embodiments, the ratio of alkanol (e.g., methanol) to supercritical liquid (e.g., carbon dioxide) is from about 2:100 to about 14:100. The ratio of alkanol (e.g., methanol) to supercritical liquid (e.g., carbon dioxide) can be about 3:100, or about 4:100, or about 5:100, or about 6:100, or about 7:100, or about 8:100, or about 9:100, or about 10:100, or about 11:100, or about 12:100, or about 13:100, or about 14:100.

In certain aspects, the extraction can be conducted in an isocratic fashion, wherein the composition of the extraction solvent remains constant throughout the extraction procedure. For example, the amounts of supercritical liquid (e.g., carbon dioxide) and alkanol (e.g., methanol) are constant over the time or extraction, for example, by maintaining a constant flow rate of each. Alternatively, the composition of the extraction solvent can be varied over time, typically by altering (e.g., increasing or decreasing) the amount of the supercritical liquid and/or alkanol components that make up the extraction solvent. Generally, the concentration of supercritical liquid (e.g., carbon dioxide) is kept constant while the concentration of alkanol (e.g., methanol) in the extraction solvent is altered (e.g., increased or decreased) over time of the extraction. The concentrations of the components can be altered by adjusting the flow rate.

In embodiments where the composition of the extraction solvent is varied over time, a method in which the second alkanol is increased as the extraction process progresses (either as a step-wise gradient or a continuously escalating gradient) is beneficial to the method. In certain instances, commercial grade poloxamers have both high molecular weight components and low molecular weight components along with the main product or component. Low alkanol (e.g., methanol) concentrations in high pressure carbon dioxide extraction fluid can selectively remove low molecular weight components. The solubility of impurity-enriched extractables, however, is low, and it takes time to significantly reduce the low molecular weight components, making it less efficient. By increasing the alkanol concentration of the extraction solvent in a gradient fashion (either step-wise gradient or as a continuously escalating gradient), the amount of extracted low molecular weight impurity increases.

Also, higher alkanol (e.g., methanol) concentrations increase the solubility, and hence extraction, of higher molecular weight components. Thus, a gradient with successively higher alkanol (e.g., methanol) concentrations of the extraction solvent can progressively extract low molecular weight components as well as eventually higher molecular weight components or components that are less soluble in lower concentrations of the extraction solvent. As a non-limiting example to illustrate this, it is believed that a lower alkanol (e.g., methanol) concentration of about 6.6% w/w can remove low molecular weight components. Increasing the concentration of alkanol by 1% to 3% will continue to effect extraction of low molecular weight components, but also result in removal of higher molecular weight components. A further increase in the concentration of alkanol by 1% to 3% will further remove these components as well as other components that have a higher molecular weight and/or are less soluble in the previous extraction solvents.

An extraction solvent with higher alkanol (e.g., methanol) concentrations is not as selective; it provides more solubility for low molecular weight components, but also increases the solubility of other components, including the main peaks. Therefore, the yield of purified product is reduced with high methanol concentrations. By increasing the concentration of the extraction solvent in a gradient fashion, as provided in methods herein, the reduction of poloxamer yield is minimized and the purity of the final product is maximized.

Increasing the methanol concentration step-wise increases the loading capacity of the extractor, thereby increasing the throughput in a given extraction system. A two-phase system forms inside the extractor. A lower phase primarily of a mixture of poloxamer and methanol with some dissolved carbon dioxide. The extraction solvent (carbon dioxide with a lower methanol co-solvent fraction) permeates through the lower phase. An upper phase contains the extraction solvent and the components extracted from the poloxamer. The relative amount of the two phases depends upon methanol concentration in the solvent flow. In a typical extraction system there is adequate head space for proper phase separation of the upper phase. Increasing the methanol co-solvent concentration step-wise during the extraction process leads to higher feed charge into the extractor.

For example, returning to process 100, the composition of the extraction solvent can be varied as shown in steps 130-140. In some embodiments, the percentage of alkanol (e.g., methanol) by weight of the extraction solvent is increased over the course of the method. The methanol content in a methanol/carbon dioxide mixture can be increased in a stepwise fashion or a continuous fashion as the extraction process progresses. In some embodiments, for example, the extraction process for a poloxamer (e.g., P188) starts using about 3% to about 10% by weight (w/w) of an alkanol (e.g., methanol) in an extraction solvent with a supercritical liquid (e.g., carbon dioxide), such as about 5% to about 10%, such as 6% to 8% (e.g., about 6.6% or 7.4%). After a defined period, the alkanol (e.g., methanol) content of the extraction solvent is raised about 1-3%, such as 1-2% (e.g., to 7.6% or 9.1%, respectively). The alkanol (e.g., methanol) content is again subsequently raised about 1-3%, such as 1-2% (e.g., to 8.6% or 10.7%, respectively) during a final period. Any suitable solvent gradient can be used in the methods of the invention. For example, the alkanol (e.g., methanol) concentration in the extraction solvent can be increased from about 5% to about 20% over the course of extraction procedure. The alkanol (e.g. methanol) concentration in the extraction solvent can be increased from about 5% to about 20%, or from about 5% to about 15%, or from about 5% to about 10%. The alkanol (e.g. methanol) concentration in the extraction solvent can be increased from about 6% to about 18%, or from about 6% to about 12%, or from about 6% to about 10%. The alkanol (e.g. methanol) concentration in the extraction solvent can be increased from about 7% to about 18%, or from about 7% to about 12%, or from about 7% to about 10%. The alkanol (e.g. methanol) concentration can be increased in any suitable number of steps. For example, the alkanol (e.g. methanol) concentration can be increased over two steps, or three steps, or four steps, or five steps over the course of the extraction procedure. A skilled artisan will appreciate that other solvent ratios and solvent gradients can be used in the extraction processes.

The time of extraction for the process provided herein can be for any defined period that results in a suitable extraction of material in the preparation while minimizing poloxamer yield reduction and maximizing purity. The time is a function of the choice of pressure, temperature, second alkanol concentration, and process of providing the extraction solvent (e.g., isocratic or as a gradient of increasing alkanol concentration as described herein). Generally, the extraction proceeds for 5 hours to 50 hours, and generally 10 hours to 30 hours, or 15 hours to 25 hours, each inclusive, such as about 15 hours or 24 hours. The higher the alkanol (e.g., methanol) concentration employed in the method, typically the shorter the time of the extraction. It also is understood that in examples in which a gradient of alkanol is employed in the method, the total time of extraction is divided as a function of the number of gradient steps in the procedure. The extraction in each gradient step can be for the same amount of time or for different times. It is within the level of a skilled artisan to empirically determine the times of extraction to be employed.

Samples can be collected during the extraction process to monitor the removal of substances or to determine if adjustment of extraction parameters, such as temperature or the composition of the extraction solvent, is necessary.

In particular, the methods can be used to purify P188. The process can be applied to other polymers as well. For example, in some embodiments, the methods provided herein provide a method for preparing a purified polyoxypropylene/polyoxyethylene composition. The method includes:

a) providing or introducing into an extractor vessel a polyoxypropylene/polyoxyethylene block copolymer that is dissolved in a first solvent to form the copolymer solution, wherein the first solvent is methanol, ethanol, propanol, butanol, pentanol or a combination thereof, and the composition contains:
i) a polyoxypropylene/polyoxyethylene block copolymer having where the mean or average molecular weight of the copolymer is from about 4000 to about 10,000 Da; and
ii) a plurality of low molecular weight substances having molecular weights of less than 4500 Da, wherein the plurality of low molecular weight substances constitutes more that 4% of the total weight of the composition;

b) adding a second solvent to form an extraction mixture, wherein the second solvent contains a supercritical liquid under high pressure and high temperature and an alkanol that is methanol, ethanol, propanol, butanol, pentanol or a combination thereof, and the concentration of the second solvent in the extraction solvent is increased over the time of extraction method; and

c) allowing the extraction mixture to separate to form a plurality of phases, including a raffinate phase and an extract phase, wherein the raffinate phase and extract phase are separately removed or isolated.

In some cases of the above method, the mean or average molecular weight of the copolymer is from about 7680 to 9510 Da, such as generally 8400 to 8800 Da, for example about or at 8400 Da. In the method, the copolymer solution can be formed in the extractor vessel by the addition of the copolymer and by adding a first solvent to form a solution or a suspension of the copolymer, wherein the first solvent is an alkanol selected from methanol, ethanol, propanol, butanol, pentanol, and a combination thereof. Alternatively, the addition of the first solvent to the copolymer to form a copolymer solution can be in a separate vessel and the copolymer solution, which is dissolved in the first solvent, is provided or introduced (i.e., charged) into the extractor vessel. In some cases, prior to step c), the method includes stirring the extraction mixture under high pressure and high temperature to extract impurities (e.g., low molecular weight extractable components and other components) from the copolymer composition.

b. Extraction Vessel and System

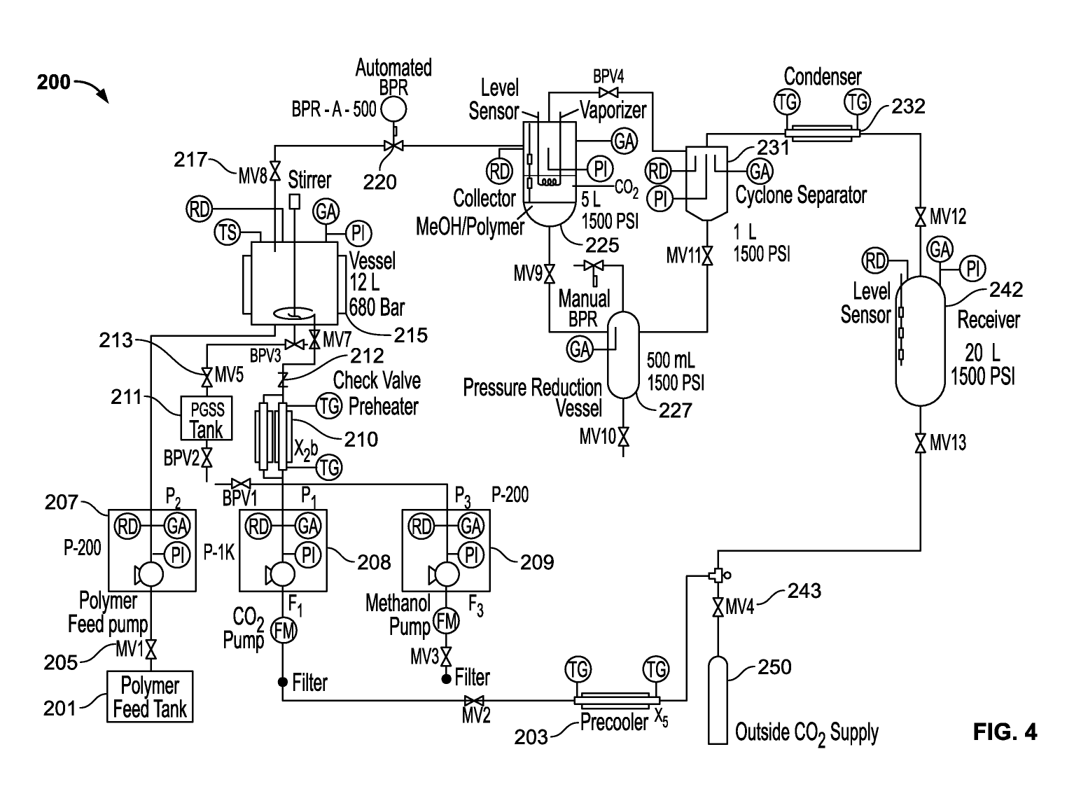

For any of the methods provided herein, turning now to FIG. 4, system 200 represents one embodiment for practice of the provided methods. System 200 is one system that can be used to extract impurities (e.g., LMW substances or other components) from the poloxamers using supercritical fluids or sub-supercritical methods.

Polymer feed pump 201 is charged with a poloxamer to be purified, for example, P188. The poloxamer is transported into polymer feed tank 207 through value 205. The extractor vessel 215 is used to remove the extracted impurities from the sample, such as LMW substances or other components from the poloxamer. Carbon dioxide (or other supercritical liquid or sub-supercritical liquid) pump 208 is charged with carbon dioxide from outside carbon dioxide supply 250 through value 243 and pre-cooler 203. Carbon dioxide is pumped from pump 208 into heat exchanger 210 and then into extractor 215. Methanol (or other suitable solvent) is pumped into extractor 215 through pump 209. In such embodiments, methanol and carbon dioxide extract impurities, such as LMW substances or other components, from the poloxamer in extractor 215. After extraction, the purified poloxamer mixture is discharged and collected via rapid depressurization processing. The extracted components are isolated from the solvent stream using collector 225, pressure reduction vessel 227, and cyclone separator 231. Carbon dioxide vapor released during collection in collector 225 can be liquefied and recycled using condenser 232.

In some embodiments, the extraction apparatus can include a solvent distribution system that contains particles of certain shapes forming a “fluidized” bed at the bottom of the extraction vessel. The bed can be supported by a screen or strainer or sintered metal disk. The particles used for the bed can be either perfectly shaped spheres or particles of irregular shape, such as pebbles. Having a smooth surface with less porosity or less surface roughness is used for easy cleaning. These advantages can be validated in a pharmaceutical manufacturing processes.

The density of the particles forming the bed is selected to be higher than the solvent density so the bed remains undisturbed by the incoming solvent flow during the extraction process. The size of the particles can be uniform or can have a distribution of different sizes to control the packing density and porosity of the bed. The packing distribution arrangement is designed to provide for balanced, optimum extraction and subsequent coalescence of the solvent particles before exiting the extraction vessel. This facilitates maximum loading of the extractor with poloxamer charge. This can also maximize extraction efficiency, minimize the extraction time, and minimize undesirable carry-over of the purified product out of the extraction vessel.

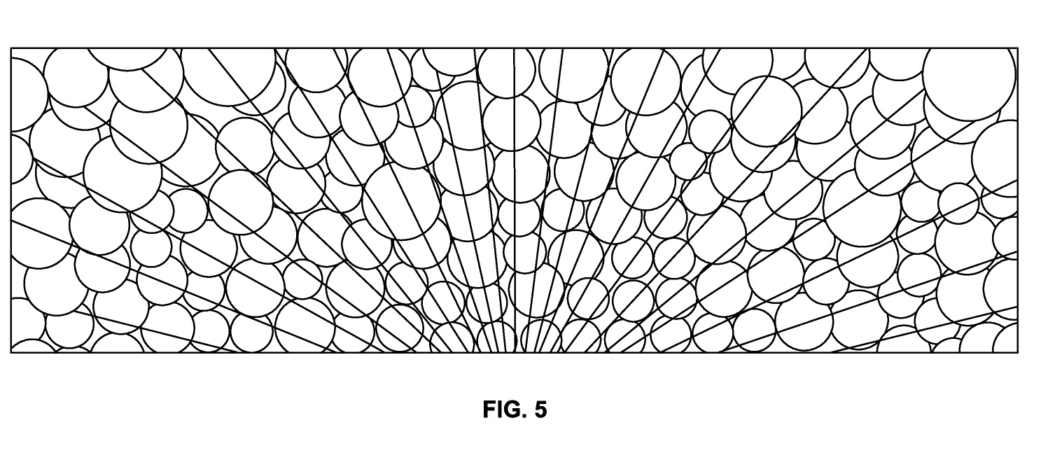

The size of the spheres in the bed is selected based on one or more system properties, including the dimensions of the extraction vessel, the residence time of the solvent droplets in the extraction vessel, and the ability of the solvent droplets to coalesce. The diameter of the spheres can range from about 5 mm to about 25 mm. The diameter can be an average diameter, wherein the bed contains spheres of different sizes. Alternatively, all of the spheres in the bed can have the same diameter. An example of the cross section of stainless steel spheres of different sizes in a solvent distribution bed is shown in FIG. 5.

Accordingly, some embodiments of the present method provide an efficient solvent extraction apparatus. The apparatus includes:

a) a distribution system at the bottom of the extractor, wherein the distribution system contains a plurality of spheres; and

b) a particle coalescence system at the top of the extractor.

In some embodiments, the plurality of spheres includes metallic spheres, ceramic spheres, or mixtures thereof. In some embodiments, the plurality of spheres are the same size. In some embodiments, the plurality of spheres include spheres of different sizes. In some embodiments, the particle coalescence system includes one or more members selected from a demister pad, a static mister, and a temperature zone.

c. Extraction and Removal of Extractants

Any of the methods provided herein can be performed as a batch method or as a continuous method.

In some embodiments, the method is a batch method. A batch method can be performed with extraction vessels of various dimensions and sizes as described above. For example, the equipment train can contain a 120 L high pressure extractor. A poloxamer (e.g., P188) solution, which is a poloxamer dissolved in an appropriate solvent (e.g., an alkanol solvent, such as methanol), is provided or introduced into the extraction vessel. The extraction solvents, such as any described in the methods above (e.g., supercritical or high-pressure carbon dioxide and methanol) are independently and continuously pumped into the extraction vessel maintained at a controlled temperature, flow, and pressure. Substances are removed by varying the extraction solvent composition as described herein. Alternatively, the extraction process conditions, such as temperature and pressure, also can be varied independently or in combination. As described below, after substances are removed, the purified product is discharged into a suitably designed cyclone separator to separate the purified product from carbon dioxide gas. The product is dried to remove the residual alkanol solvent.

In some embodiments, the extraction method is a continuous method. In a typical continuous extraction, a poloxamer (e.g., P188) solution, which is a poloxamer dissolved in an appropriate solvent (e.g., an alkanol solvent, such as methanol), is loaded at the midpoint of a high pressure extraction column packed with a suitable packing material. The extraction solvent is pumped through the extraction column from the bottom in counter-current fashion. The extracted material, such as LMW substances or other components, is removed at the top of the column, while purified product is removed from the bottom of the column. The purified product is continuously collected at the bottom of the extractor column and periodically removed and discharged into a specially designed cyclone separator. The purified polymer particles containing residual methanol are subsequently dried under vacuum.

Depending on the level of purity desired in the purified poloxamer product, the extraction step can be repeated for a given batch. Additional portions of the extraction solvent can be introduced into the extractor vessel and removed until a sufficient level of poloxamer purity is obtained. Accordingly, some embodiments of methods provided herein provide extraction methods as described above, wherein after step c), the method further includes repeating steps b) and c). Steps b) and c) can be repeated until the poloxamer is sufficiently pure. For example, steps b) and c) can be repeated one time, two times, three times, four times, or five times, or in an iterative fashion.

When the poloxamer material is sufficiently pure, the product is prepared for further processing. In some embodiments, the product is handled according to process 100 as summarized in FIG. 1. The product can be discharged from the extractor vessel and collected in an appropriate receiver, as shown in step 145. The wet product can be sampled for testing with respect to purity, chemical stability, or other properties, as shown in step 150. The product can be dried by removing residual solvents under vacuum. The vacuum level can be adjusted to control drying rates. Drying can be conducted at ambient temperature, or at elevated temperatures if necessary. In general, the drying temperature is held below the melting point of the poloxamer. The wet product can be dried in a single lot or in smaller portions as sub-lots. As shown in steps 160-170, drying of the product can be initiated, for example, on a sub-lot, under vacuum, at ambient temperature. Drying can then be continued at higher temperatures and lower pressures as the process progresses. If necessary, for example, if collection was made in sub-lots, any remaining portions of the wet product can be processed in a similar manner, as shown in step 175 of process 100. The resulting product, such as the various sub-lots that have been combined, are mixed in a suitable container, as shown in step 180, and the resulting product can be characterized, stored, transported, or formulated.

Advantageously, the methods disclosed herein effectively recycle carbon dioxide. In particular, supercritical carbon dioxide or high-pressure carbon dioxide can be recovered by subjecting the extract phase to changes in temperature and pressure. In certain embodiments, the methods employed herein have recycling efficiencies of greater than 80%, greater than 90%, or greater than 95%.

In any of such methods, the methods provided herein can further include: d) passing the extract phase to a system that includes several separation vessels; g) isolating the impurities (e.g. low molecular-weight impurities); h) processing the purified material or raffinate and i) recovering the compressed carbon dioxide for reuse.

In any of the methods provided herein, various parameters can be assessed in evaluating the methods and resulting products. For example, parameters such as methanol concentration, gradient profile, temperature, and pressure can be assessed for process optimization. Processes and suitable conditions for drying wet raffinate, such as vacuum level, mixing mode, time, and temperature, also can be assessed.

d. Exemplary Methods

The methods provided herein above result in the generation of particular purified poloxamer preparations and in particular P188 preparations. In particular, the methods provided herein can be used to purify a P188 copolymer as described herein that has the formula HO(CH₂CH₂O)_{a′}—[CH(CH₃)CH₂O]_{b}—(CH₂CH₂O)ₐH, an average or mean molecular weight of the copolymer from 7680 to 9510 Da, such as generally 8400 to 8800 Da, for example about or at 8400 Da, and contains a plurality of low molecular weight substances having a molecular weight of less than 4000 Da, wherein the plurality of low molecular weight substances constitutes more that 4% of the total weight of the composition.

In some embodiments, the present methods generate purified poloxamers with less than about 5% low molecular weight components such as less than about 4%, 3%, 2% or 1% low molecular weight components. Typically, the low molecular weight components include glycols and volatile degradation impurities such as formaldehyde, acetaldehyde, propionaldehyde, acetone, methanol, and peroxides. In certain instances, the processes herein produce poloxamers substantially free of low molecular weight components, i.e., less than 5%, 4%, 3%, 2% or 1% of the foregoing components. The methods also can produce poloxamers substantially free of long circulating material such that when the purified poloxamer is administered to a subject there are no components in the poloxamer that are or give rise to a material that has a longer half-life in the blood or plasma more than 5.0-fold the half-life of the main peak in the poloxamer distribution, such as generally no more than 4.0-fold, 3.0-fold, 2.0-fold, or 1.5-fold. Exemplary of such methods that produce these purified products are described below.

i. Removal of Low Molecular Weight (LMW) Components

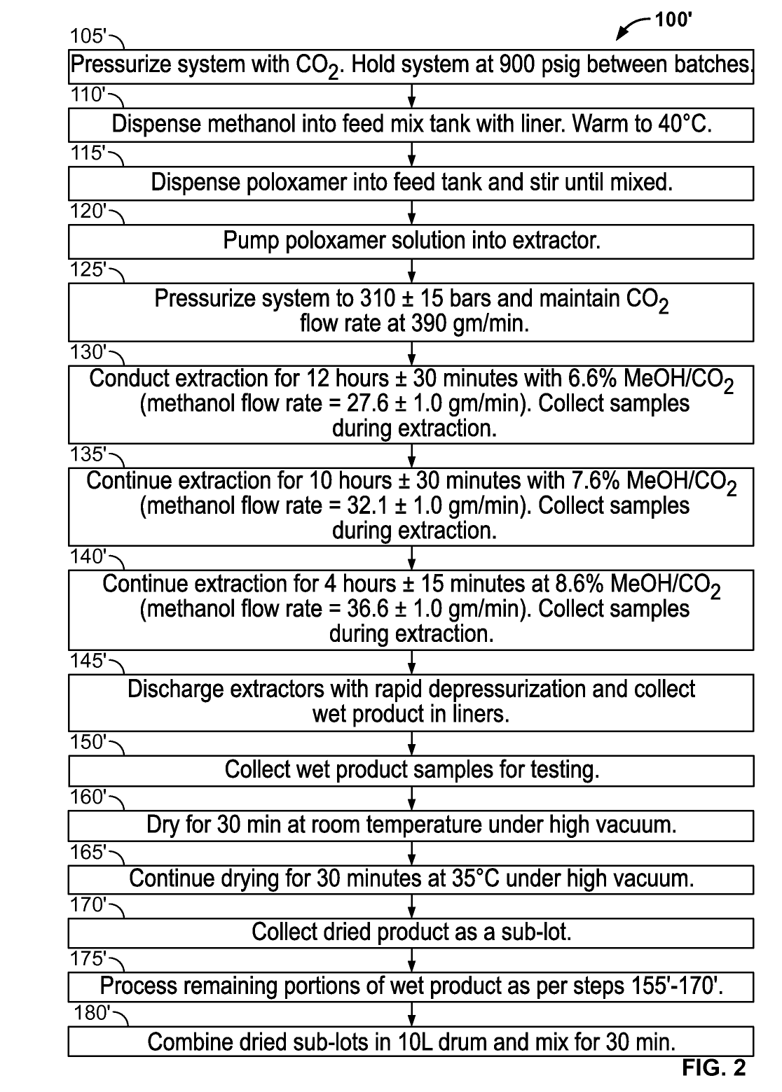

FIG. 2 depicts certain embodiments of the methods herein provide a process 100′ that is useful for removing low molecular weight (LMW) substances in a poloxamer. The extraction system is pressurized, as shown in step 105′, prior to dispensing a first alkanol (e.g., methanol) into the feed mix tank, as shown in step 110′. The system is heated to a temperature suitable for the extraction process, which is a temperature above the critical temperature of carbon dioxide (used in the process), i.e., about 31° C. Typically, the temperature is no more than 40° C. The temperature is generally kept constant through the process.

The first alkanol (e.g., methanol) is used to form a poloxamer solution according to step 115′ in process 100′. In this process, dispensing a P188 poloxamer into the feed tank with the alkanol (e.g., methanol), results in a P188 poloxamer solution that is dissolved in the alkanol (e.g., methanol). The amount of poloxamer for use in the method can be any amount, such as any amount described herein above. After forming a poloxamer/alkanol mixture, all or part of the mixture is pumped into the extractor as shown in step 120′. In some cases, the poloxamer solution can be formed in the extraction vessel by introducing the poloxamer as a solid into the extractor prior to mixing with the alkanol.

The extractor is then pressurized and the extraction solvent is introduced into the extractor as shown in step 125′ of process 100′. The extraction solvent typically contains carbon dioxide and the extraction is performed at a temperature greater than the critical temperature of 31° C., as described above, and under high pressure, i.e., greater than the critical pressure of 74 bars. For example, in an exemplary method, the extraction vessel is pressurized to about 310±15 bars, and the carbon dioxide is provided at a flow rate that is 20 kg/h to 50 kg/h, such as generally about or approximately 24 kg/h (i.e., 390 g/min).

The extraction is then conducted in the presence of a second alkanol acting as a co-solvent modifier of the carbon dioxide. The second alkanol, such as methanol, is added in a gradient step-wise fashion such that the concentration of the second alkanol in the extraction solvent is increased over the time of extraction method. For example, the composition of the extraction solvent can be varied, as shown in steps 130′-140′. For example, as shown in step 130′, the extraction process for a poloxamer (e.g., P188) initially uses about 5% to 7%, by weight (w/w) of an alkanol (e.g., methanol) in an extraction solvent with a supercritical liquid (e.g., carbon dioxide), for example, about 6.6%. After a defined period, the alkanol (e.g., methanol) content of the extraction solvent is raised about 1-3%, such as 1% (e.g., to 7.6%). The alkanol (e.g., methanol) content is again subsequently raised about 1-3%, such as 1% (e.g., to 8.6%) during a final period. The total time of the extraction method can be 15 hours to 25 hours. Each gradient is run for a portion of the total time.

For a commercially viable and efficient purification process, it is desirable to have successively increasing methanol concentrations where the profile is suitably modified to selectively remove most of the LMW components. Residual LMW components can be subsequently removed with high methanol concentrations in a short time. Therefore, a step-wise methanol concentration profile, where a concentration of about 5% to 10% (e.g., 6.6%) methanol is used for 12 hours, a higher methanol concentration is used for 10 hours, and finally an even higher methanol concentration is used for 4 hours, produces purified product in high yields without significantly reducing the overall yield and not enriching the high molecular weight components.

When the poloxamer material is sufficiently pure, the product is prepared for further processing as shown in process 100′. The product can be discharged from the extractor vessel and collected in an appropriate receiver, as shown in step 145′. The wet product can be sampled for testing with respect to purity, chemical stability, or other properties, as shown in step 150′. The product can be dried by removing residual solvents under vacuum as described herein. In an exemplary method, as shown in steps 160′-170′, drying can be initiated with a sub-lot under vacuum at ambient temperature and drying can be continued at higher temperatures and lower pressures as the process progresses. Remaining sub-lots can be processed in a similar manner, as shown in step 175′ of process 100′. Sub-lots can be combined and mixed in a suitable container, as shown in step 180′, and the resulting product can be characterized, stored, transported, or formulated.

ii. Preparation of Longer Circulating Material Free (LCMF) Poloxamer

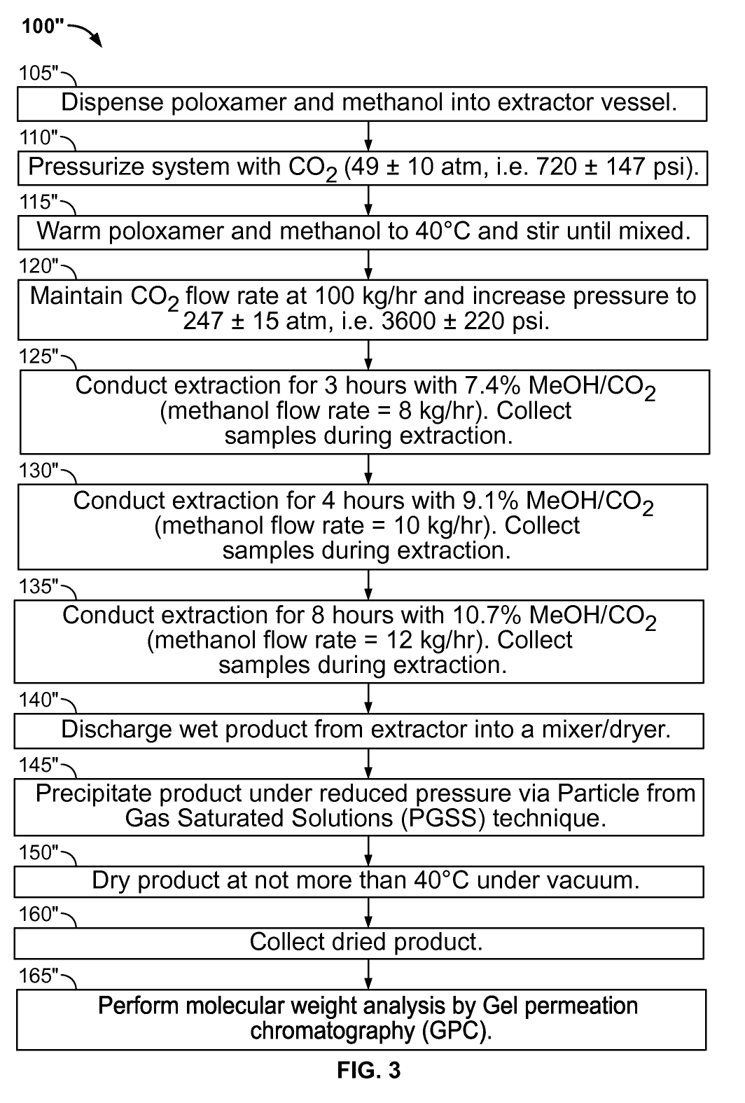

Turning now to FIG. 3, certain embodiments of the methods herein provide a process 100″ that is useful for generating a poloxamer that does not contain any components that, after administration to a subject, result in a long circulating material in the plasma or blood as described herein. As shown in step 105″, the poloxamer and first alkanol (e.g., methanol) are dispensed into the extractor vessel and form the poloxamer solution. In this process, dispensing a P188 poloxamer into the extraction vessel with the alkanol (e.g., methanol) results in a solution where the P188 poloxamer is dissolved in the alkanol (e.g., methanol). The amount of poloxamer for use in the method can be any amount as described herein. In some cases, the poloxamer solution can be formed in a separate vessel, and the poloxamer solution transferred to the extractor vessel.

The extraction system is pressurized, as shown in step 110″, after dispensing a first alkanol (e.g., methanol) and poloxamer. As shown in step 115″, the system is heated to a temperature suitable for the extraction process, which is a temperature above the critical temperature of carbon dioxide (used in the process), i.e., about 31° C. Typically, the temperature is no more than 40° C. The temperature is generally kept constant through the process. The poloxamer solution is formed under pressurized carbon dioxide, e.g., about 49 bars, and a temperature of no more than 40° C. or about 40° C. for a defined period, generally less than several hours.

The extractor is then pressurized and the extraction solvent is introduced into the extractor as shown in step 120″ of process 100″′. The extraction solvent typically contains carbon dioxide and a second alkanol, and extraction is performed at a temperature greater than the critical temperature of 31° C., as described above, and under high pressure, i.e., greater than the critical pressure of 74 bars. For example, in an exemplary method, the extraction vessel is pressurized to about 247±15 bars, and the carbon dioxide is provided at a flow rate that is 50 kg/h to 100 kg/h, inclusive, such as generally about or approximately 100 kg/h.

The extraction is conducted in the presence of the second alkanol, which acts as a co-solvent modifier of the carbon dioxide. As shown in steps 125″-135″, the second alkanol, such as methanol, is added in a gradient step-wise fashion, such that the concentration of the second alkanol in the extraction solvent is increased over the time of extraction method. For example, the composition of the extraction solvent can be varied as shown in steps 125″-135″. For example, as shown in step 125″, the extraction process for a poloxamer (e.g., P188) initially uses about 7% to 8%, by weight (w/w) of an alkanol (e.g., methanol) in an extraction solvent with a supercritical liquid (e.g., carbon dioxide), for example, about 7.4%. After a defined period, the alkanol (e.g., methanol) content of the extraction solvent is raised about 1-3%, such as up 2% (e.g., to 9.1%). The alkanol (e.g., methanol) content is again subsequently raised about 1-3% such as up 2% (e.g., to 10.7%) during a final period. The total time of the extraction method can be 15 hours to 25 hours, inclusive. Each gradient is run for a portion of the total time.

For an extraction process that removes components other than LMW components, including components that, when administered, give rise to longer circulating forms, it is desirable to have a process that maximizes the purity and removal of these components while minimizing loss in yield. It is found that successively increasing the alkanol (e.g., methanol) concentration, starting from a higher concentration of alkanol (e.g. methanol) than in other methods, generally starting at 7% to 8% by weight, suitably modifies the profile to selectively remove these components and low molecular weight components, while minimizing reductions in yield. For example, such an exemplary method can produce yields greater than 55%, and generally greater than 60% or 65%. Residual low molecular weight components can subsequently be removed with high methanol concentrations in a short time. Therefore, a stepwise methanol concentration profile, where about a 7-8% (e.g., 7.4%) methanol concentration is used for about 3 hours, a higher methanol concentration (e.g., 9.1%) is used for about 4 hours, and finally, an even higher methanol concentration (e.g., 10.7%) is used for about 8 hours, produces purified product in high yields without significantly reducing the overall yield.

When the poloxamer material is sufficiently pure, the product is prepared for further processing as shown in process 100″. The product can be discharged from the extractor vessel and collected in an appropriate receiver, as shown in step 140″. The product can be precipitated under reduced pressure via the particles from gas saturated solutions (PGSS) technique as shown in step 145″. The product can be dried by removing residual solvents under vacuum as described herein. In an exemplary method, as shown in steps 150″-165″, drying can be initiated under vacuum at high temperatures no more than 40° C. The dried product can be collected as shown in step 160″. The resulting product can be characterized, stored, transported, or formulated as shown in step 165″.

### D. PHARMACEUTICAL COMPOSITIONS AND FORMULATIONS

Compositions containing a poloxamer described herein, including any prepared by methods described herein and/or known to those of skill in the art, are provided. The compositions containing an LCMF poloxamer are provided. The concentration of poloxamer is such that it achieves a target plasma concentration for a time sufficient to effect treatment. The particular time and concentration depends upon the target plasma concentration, the mode of administration, the duration of administration, and the regimen. The skilled artisan can prepare such compositions. As described the poloxamer can be administered alone or in combination with other agents, for example, diuretics. The compositions can be co-formulated or administered. Exemplary compositions are their use are described in Section F.

1. Formulations

Pharmaceutical compositions containing polyoxyethylene/polyoxypropylene copolymer, such as a P188, including a LCMF P188, can be formulated in any conventional manner by mixing a selected amount of the poloxamer with one or more physiologically acceptable carriers or excipients. Selection of the carrier or excipient is within the skill of the art and can depend upon a number of parameters. These include, for example, the mode of administration (i.e., systemic, oral, nasal, pulmonary, local, topical, or any other mode) and the symptom, side effect, disorder, or disease to be treated.

Concentrations of the poloxamer, such P188, such as an LCMF P188, are mixed with a suitable pharmaceutical carrier or vehicle for systemic, topical or local administration. In particular, for the methods herein, the poloxamer is administered IV, such as by continuous infusion or a series of bolus injections.

Pharmaceutical carriers and vehicles suitable for administration of the copolymers include any such carriers known to those skilled in the art to be suitable for the particular mode of administration. Pharmaceutical compositions can be provided as a lyophilized powder that is reconstituted, such as with sterile water, immediately prior to administration.

The compositions can be prepared for dilution prior to administration or for direct administration. In general, for the methods herein, the compositions are administered by IV, either continuous infusion or a series of bolus injections. The target circulating concentration is at least 0.5 mg/ml, and can be as high as 15 mg/ml, but generally is up to and including 1.5 mg/ml or 2 mg/ml. This level is maintained for a sufficient number of hours to effect treatment, typically at least 12 hours to 1-3 days or 4 days to reduce or eliminate undesirable effects and complications of the hemo-concentration, dehydration and/or diuresis or to prevent the risk of developing such effects/complications.

The poloxamer can be suspended in micronized form or other suitable form or can be derivatized to produce a more soluble active product. The form of the resulting mixture depends upon a number of factors, including the intended mode of administration and the solubility of the particular poloxamer, such as P188, such as LCMF P188, in the selected carrier or vehicle. The resulting mixtures are solutions, suspensions, emulsions and other such mixtures, and can be formulated as non-aqueous or aqueous mixtures, creams, gels, ointments, emulsions, solutions, elixirs, lotions, suspensions, tinctures, pastes, foams, aerosols, irrigations, sprays, suppositories, bandages, or any other formulation suitable for systemic, topical or local administration. For purposes herein, the compositions typically are aqueous solutions suspensions or emulsions for IV administration.

Generally, pharmaceutically acceptable compositions are prepared in view of approvals from a regulatory agency or are prepared in accordance with generally recognized pharmacopeia standards for use in animals and in humans. For example, the methods provided herein have applications for both human and animal use.

Pharmaceutical compositions can include carriers such as a diluent, adjuvant, excipient, or vehicle with which an isoform is administered. Such pharmaceutical carriers can be sterile liquids, such as water and oils, including petroleum, animal, vegetable, or those of synthetic origin, such as peanut oil, soybean oil, mineral oil, and sesame oil. Water and saline solutions are typical carriers when the pharmaceutical composition is administered intravenously. Saline solutions and aqueous dextrose and glycerol solutions also can be employed as liquid carriers, particularly for injectable solutions. Liposomal suspensions, including tissue-targeted liposomes, also can be suitable as pharmaceutically acceptable carriers. These can be prepared according to methods known to those skilled in the art. For example, liposome formulations can be prepared as described in U.S. Pat. No. 4,522,811. Liposomal delivery also can include slow release formulations, including pharmaceutical matrices, such as collagen gels and liposomes modified with fibronectin (see, for example, Weiner et al. (1985) J. Pharm. Sci. 74(9):922-925).

Compositions can contain, along with poloxamer, such as P188, such as LCMF P188: a diluent, such as lactose, sucrose, dicalcium phosphate, and carboxymethylcellulose; a lubricant, such as a stearate, such as, calcium stearate, and talc; and a binder, such as starch, natural gums, such as gum acacia gelatin, glucose, molasses, polyvinylpyrrolidine, celluloses and derivatives thereof, povidone, crospovidones, and other such binders known to those of skill in the art. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, and ethanol. A composition, if desired, also can contain small amounts of wetting or emulsifying agents, and/or pH buffering agents, for example, acetate, sodium citrate, cyclodextrin derivatives, sorbitan monolaurate, triethanolamine sodium acetate, triethanolamine oleate, and other such agents. For purposes herein, these compositions can take the form of solutions, suspensions, emulsions for IV administration. A composition can be formulated as a suppository, with traditional binders and carriers such as triglycerides. Examples of suitable pharmaceutical carriers are described in “Remington's Pharmaceutical Sciences,” E. W. Martin (ed.), Mack Publishing Co., Easton, Pa., 19^{th} Edition (1995). Such compositions will contain a therapeutically effective amount of P188, in a form described herein, including the LCMF form, together with a suitable amount of carrier so as to provide the form for proper administration to a subject or patient.

The compositions provided herein further can contain one or more adjuvants that facilitate delivery, such as, but not limited to, inert carriers or colloidal dispersion systems. Representative and non-limiting examples of such inert carriers are water, isopropyl alcohol, gaseous fluorocarbons, ethyl alcohol, polyvinyl pyrrolidone, propylene glycol, a gel-producing material, stearyl alcohol, stearic acid, spermaceti, sorbitan monooleate, and methylcellulose, as well as suitable combinations of two or more thereof.

The formulation is selected to suit the mode of administration. For example, compositions containing the poloxamer, such as P188, such as LCMF P188, can be formulated for parenteral administration by injection (e.g., by bolus injection or continuous infusion). The injectable compositions can take such forms as suspensions, solutions or emulsions in oily or aqueous vehicles. The sterile injectable preparation also can be a sterile injectable solution or suspension in a non-toxic parenterally-acceptable diluent or solvent, for example, as a solution in saline, such as citrate buffered saline. Sterile, fixed oils can be employed as a solvent or suspending medium. For this purpose any bland fixed oil can be employed, including, but not limited to, synthetic mono- or diglycerides, fatty acids (including oleic acid), naturally occurring vegetable oils like sesame oil, coconut oil, peanut oil, cottonseed oil, and other oils, or synthetic fatty vehicles like ethyl oleate. Buffers, preservatives, antioxidants, and the suitable ingredients can be incorporated as required, or, alternatively, can comprise the formulation.

Formulations suitable for parenteral administration include aqueous and non-aqueous sterile injection solutions, which can contain anti-oxidants, buffers, bacteriostats and solutes that render the formulation compatible with the intended route of administration. The formulations can be prepared in unit-dose or multi-dose form by conventional pharmaceutical techniques, for example, including bringing the active ingredient, e.g., P188, such as LCMF P188, into association with the pharmaceutical carrier(s) or excipient(s). The formulations can be presented in unit-dose or multi-dose containers, for example, sealed ampules and vials, prefilled syringes or other delivery devices, and can be stored in an aqueous solution or in a dried or freeze-dried (lyophilized) condition, requiring only the addition of the sterile liquid carrier, for example, water or saline for injection, immediately prior to use.

The poloxamer, such as P188, such as LCMF P188, can be formulated as the sole pharmaceutically active ingredient in the composition or can be combined with other active ingredients. The P188, such as LCMF P188, is included in the pharmaceutically acceptable carrier in an amount sufficient to exert a therapeutically useful effect in the absence of undesirable side effects on the subject treated. The therapeutically effective concentration can be determined empirically by testing the compounds in known in vitro and in vivo systems, such as the assays provided herein.

2. Dosage

The pharmaceutical compositions containing P188, such as LCMF P188 provided herein, can be formulated for single dosage (direct) administration, multiple dosage administration, or for dilution or other modification. Typically, the compositions containing poloxamer P188, such as LCMF P188 provided herein, are formulated to achieve a targeted circulating concentration of poloxamer, e.g., LCMF P188, in the circulation of the subject at a desired time point after administration. This target for the uses and methods herein is at least 0.05 mg/ml, typically, 0.5-1.5 mg/ml, or higher, such as up to 10 or 15 mg/ml. The desired time for such concentration is several hours, include 12 hours up to several days, 1, 2, 3 or 4 days. The timing and particular concentration depends upon the subject, the condition treated, underlying conditions and other such parameters.

Those of skill in the art readily can formulate a composition for administration in accord with the methods herein. For example, to formulate a composition, the weight fraction of a compound or mixture thereof is dissolved, suspended, dispersed, or otherwise mixed in a selected vehicle at an effective concentration such that side effects associated with diuresis are improved. The precise amount or dose of the poloxamer administered to achieve the targeted concentration can be readily determined by one of skill in the art and will depend on the route of administration, and other considerations, such as the severity of the side effect to be treated, the weight and general state of the subject, and the subject. Routine procedures that adjust for physiological variables (including, but not limited to, kidney and liver function, age, and body weight and or body surface area) can be used to determine appropriate dosing regimens. Local administration of the therapeutic agent will typically require a smaller dosage than any mode of systemic administration, although the local concentration of the therapeutic agent can, in some cases, be higher following local administration than can be achieved with safety upon systemic administration.

If necessary, a particular dosage and duration and treatment protocol can be empirically determined or extrapolated. For example, exemplary doses of the poloxamer, such s P188, such as LCMF P188 provided herein, if necessary, can be used as a starting point to determine appropriate dosages for a particular subject and condition. The duration of treatment and the interval between injections will vary with the severity of the side effect or condition and the response of the subject to the treatment, and can be adjusted accordingly. Factors such as the level of activity and half-life of the poloxamer, such as P188, such as LCMF P188, can be taken into account when making dosage determinations. Particular dosages and regimens can be empirically determined by one of skill in the art.

In particular, the poloxamer can be formulated at a concentration ranging from about 10.0 mg/mL to about 300.0 mg/mL or 10.0 to 200.0 mg/mL, such as at or at least 10.0, 15.0, 20.0, 25.0, 30.0, 35.0, 40.0, 45.0, 50.0, 55.0, 60.0, 65.0, 70.0, 75.0, 80.0, 85.0, 90.0, 95.0, 100.0, 105.0, 110.0, 115.0, 120.0, 125.0, 130.0, 135.0, 140.0, 145.0, 150.0, 155.0, 160.0, 165.0, 170.0, 175.0, 180.0, 185.0, 190.0, 195.0 or 200.0 mg/mL, for direct administration. Typically, the concentration is not more than 22.5%, i.e., 225 mg/mL. The selected amount to administer can be determined for a particular target plasma concentration and duration.

For example, when administered separately or as a component of the pharmaceutical composition described herein, the poloxamer is administered at a concentration of between about 0.5% to 20% although more dilute or higher concentrations can be used. For example, the poloxamer can be administered in an amount between about 0.5% to about 20% by weight/volume, such as 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10.0%, 10.5%, 11%, 11.5%, 12%, 12.5%, 13%, 13.5%, 14%, 14.5%, 15%, 15.5%, 16%, 16.5%, 17%, 17.5%, 18%, 18.5%, 19%, 19.5% or 20% by weight/volume. In other embodiments, the poloxamer is administered in an amount between about 0.5% to about 10% by weight/volume, such as 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, or 10.0% by weight/volume. In yet other embodiments, the poloxamer is administered in an amount between about 5% to about 15% by weight/volume, such as 5%, 5.5%, 6%, 6.5%, 7%, 7.5%, 8%, 8.5%, 9%, 9.5%, 10.0%, 10.5%, 11%, 11.5%, 12%, 12.5%, 13%, 13.5%, 14%, 14.5%, or 15% by weight/volume. For example, the concentration is 10% to 22.5%, such as 10% to 20% or 15% to 20%.

Typically, the poloxamer is formulated so that administration of the poloxamer to a subject results in an effective amount of poloxamer, such as a P188, such LCMF P188, in the circulation of the subject. The effective amount of poloxamer, such as a P188, or a LCMF P188, can be administered alone or in combination with other agents, for example, diuretics. The effective amount can be the result of administration of the poloxamer one time or multiple times, such as two, three, four, five, or more times, by various routes of administration. For example, the poloxamer, e.g., P188 or LCMF P188, is formulated so that administration of the poloxamer to a subject results in a concentration of the poloxamer in the circulation of the subject of about 0.05 mg/mL to about 15.0 mg/mL, such as about 0.05 mg/mL to about 10.0 mg/mL, about 0.5 mg/mL to about 2 mg/mL, for example, from about 0.2 mg/mL to about 4.0 mg/mL. The concentration of the poloxamer in the circulation of the subject can be representative of a single time point, or representative of a mean steady state concentration that is maintained for a period of time, for example, a period of time up to about 72 hours, such as at least about 1 hour, 2 hours, 3 hours, 4 hours, 5 hours, 10 hours, 15 hours, 20 hours, 30 hours, 40 hours, 50 hours, 60 hours, 70 hours, or more. In some examples, the target concentration of the poloxamer in the circulation is generally maintained for about 4 hours to about 72 hours, or longer.

In some examples, the poloxamer, such as a P188, such as LCMF P188, is formulated so that administration of a single dose of the poloxamer to a subject results in an effective amount of poloxamer in the circulation of the subject. For example, administration of a single dose of poloxamer, results in a concentration of the poloxamer in the circulation of the subject of about 0.05 mg/mL to about 15.0 mg/mL, or about 0.05 mg/mL to about 10.0 mg/mL, or about 0.5 mg/mL to about 2 mg/mL, for example, from about 0.2 mg/mL to about 4.0 mg/mL. For example, the concentration of the poloxamer in the circulation of the subject is from about 0.2 mg/mL to about 4.0 mg/mL, such as 0.5 mg/mL to about 2.0 mg/mL, e.g., about 0.5, 0.6, 0.7, 0.8, 0.9, 1.0, 1.1, 1.2, 1.3, 1.4, 1.5, 1.6, 1.7, 1.8, 1.9, 2.0, 2.5, 3.0, 3.5 or 4.0 mg/mL. In one example, administration of a single dose of poloxamer, e.g., a P188, or LCMF P188, results in a concentration of the poloxamer in the circulation of the subject of about 0.5 mg/mL.

In other examples, the poloxamer is formulated so that repetitive administration of the poloxamer to a subject, for example, administration a second, third, or multiple times, results in an effective amount of poloxamer in the circulation of the subject. For example, the repetitive treatment is sufficient to result in a concentration of the poloxamer in the circulation of the patient of from about 0.05 mg/mL to about 15.0 mg/mL, or about 0.05 mg/mL to about 10.0 mg/mL, or about 0.5 mg/mL to about 2 mg/mL, for example, from about 0.2 mg/mL to about 4.0 mg/mL. For example, the concentration of the poloxamer, such as LCMF P188, in the circulation of the subject is from about 0.2 mg/mL to about 4.0 mg/mL, such as 0.5 mg/mL to about 2.0 mg/mL, e.g., about 0.5, 0.6, 0.7, 0.8, 0.9, 1.0, 1.1, 1.2, 1.3, 1.4, 1.5, 1.6, 1.7, 1.8, 1.9, 2.0, 2.5, 3.0, 3.5 or 4.0 mg/mL. In one example, repetitive administration of poloxamer, e.g., LCMF P188, results in a concentration of the poloxamer in the circulation of the subject of about 0.5 mg/mL.

In one example, the poloxamer can be formulated as a sterile, non-pyrogenic solution intended for administration with or without dilution. The final dosage form can be prepared in a 100 mL vial, where the 100 mL contains 15 g of a P188, such as purified poloxamer 188 (150 mg/mL) or LCMF P188, 308 mg sodium chloride USP, 238 mg sodium citrate USP, 36.6 mg citric acid USP, and water for injection USP, q.s. (quantity sufficient) to 100 mL. The pH of the solution is approximately 6.0 and has an osmolarity of about 312 mOsm/L. The solution is sterilized prior to administration to a subject. For other applications, at least 500 mL is prepared with a concentration of 10% to 20%, such as about or at 15%, weight of poloxamer preparation/volume of the composition.

This dosage ranges provided herein are not intended to be limiting, and will vary based on the needs and response of the individual subject, the particular subject, as well as the properties of the particular poloxamer chosen for administration.

3. Administration

In the methods herein, the poloxamer, such as a purified poloxamer 188 or LCMF described herein, is administered to a subject for treating the side effects or complications of hemo-concentration. These side-effects can be associated with diuresis, and in particular any side effect or consequence associated with diuretic-induced diuresis as described in Section F. In particular, poloxamer 188, such as a purified poloxamer 188 and LCMF described herein, is intended for use in methods in which administration of diuretics, such as known diuretic therapies, for ameliorating conditions such as kidney-related conditions, high blood pressure, liver conditions, heart-related conditions, and glaucoma, results in diuresis and subsequently causes unwanted side effects or consequences. The poloxamer 188 provided herein is used tread the complications of hemo-concentration, such as to treat conditions or side effects associated with diuresis, such as electrolyte imbalance, dehydration, arrhythmia, alterations of plasma volume, hemo-concentration of blood plasma proteins and/or blood cells, and any other side effect or unwanted consequence associated with diuresis.

Treatment of side effects and conditions associated with diuresis, such as any described in Section F, with poloxamer 188, such as a purified poloxamer 188 and LCMF described herein, can be effected by any suitable route of administration using suitable formulations as described herein including, but not limited to, injection, pulmonary, oral and transdermal administration. Treatment typically is effected by intravenous administration.

Active agents, for example a poloxamer 188, such as an LCMF P188, are included in an amount sufficient to exert a therapeutically useful effect in the absence of undesirable side effects on the patient treated. Generally, a therapeutically effective amount of poloxamer results in a concentration of the poloxamer in the circulation of the subject, i.e., a targeted plasma concentration, of between about 0.05 mg/mL and about 15.0 mg/mL in the subject, e.g., about 0.05, 0.06, 0.07, 0.08, 0.09, 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0, 10.5, 11.0, 11.5, 12.0, 12.5, 13.0, 13.5, 14.0, 14.5, or 15.0 mg/mL and the concentrations described elsewhere herein.

The amount of a poloxamer, such as P188, such as a LCMF P188, to be administered for the treatment of complications of hemo-concentration, such for example, the side effects of diuresis. They can be administered, for example, for treating a subject with acute dehydration or hemo-concentration from other causes. the need for such treatment, if not readily apparent, can be determined by standard clinical techniques. In addition, if needed, in vitro assays and animal models can be employed to help identify optimal dosage ranges. The precise dosage, which can be determined empirically, can depend on the particular composition, the route of administration, the type of side effect to be treated and the seriousness of the side effect.

As described elsewhere herein, poloxamer 188 described been modified by purification to remove LMW components. It is known that removal of these components, prevents elevation of creatine levels and renal toxicity observed with preparation so of P188 that included the LMW contaminants. In clinical studies, for example the C97-1248 study, where creatine levels were evaluated in human patients treated with a purified form of poloxamer 188 (P188-P), which lacks a lower molecular weight species of poloxamer 188, researchers found that intravenous administration of P188-P failed to induce a significant increase in serum creatine above the levels of a placebo. The loss of low and high molecular weight species, based on assessment by high performance liquid chromatography, reduces or eliminates renal risk associated with unpurified (P188-NF) treatments. Therefore, a purified poloxamer 188, such as a poloxamer 188 described herein, does not exhibit the practical limitations present in the previously assessed, unpurified form. The dosing regimen of a poloxamer, such as a purified poloxamer 188 or LCMF 188 described herein, has been modified to address the limitations of clinical use of previous poloxamer 188.

In some examples, methods of treatment with poloxamer 188 require a longer duration of action in order to effect a sustained therapeutic effect. As discussed elsewhere herein, the half-life of poloxamer 188 is 18 hours. Despite the relatively short half-life, the effects of a poloxamer such as a purified poloxamer 188, can be long lasting. Thus, the poloxamer 188 described herein can be used to deliver longer lasting therapies for the treatment of the complications of hemo-concentration, including side effects of diuresis and dehydration. In general the poloxamer is administered IV to achieve and maintain a target concentration of at least 0.5 mg/ml or other target concentration for sufficiently long to effect treatment. This includes at least 12 hours, 1 day 2, days, 3 days, and up to 4 days.

If necessary, a particular dosage and duration and treatment protocol can be empirically determined or extrapolated. Dosages for poloxamer, including poloxamer 188 previously administered to human subjects and used in clinical trials can be used as guidance for determining dosages for poloxamer 188, such as a purified poloxamer 188 and LCMF 188 described herein. Dosages for poloxamer 188 if necessary, also can be determined or extrapolated from relevant animal studies. Factors such as the level of activity and half-life of poloxamer 188 can be used in making such determinations. Particular dosages and regimens can be empirically determined based on a variety of factors. Such factors include body weight of the individual, general health, age, the activity of the specific compound employed, sex, diet, time of administration, rate of excretion, drug combination, the severity and course of the side effects, and the patient's disposition to the side effects and the judgment of the treating physician. The active ingredient, poloxamer 188, typically is combined with a pharmaceutically effective carrier. The amount of active ingredient that can be combined with the carrier materials to produce a single dosage form or multi-dosage form can vary depending upon the host treated and the particular mode of administration.

In particular examples, the poloxamer, such as P188 such as LCMF 88), is formulated for administration to a subject at a dosage of 25 mg to 675 mg/kg, including for 25-50 mg/kg; 100 to 675 mg/kg, such as 100 to 500 mg/kg subject body weight, for example, 100 mg/kg to 450 mg/kg, 100 to 400 mg/kg, 100 mg/kg to 300 mg/kg, 100 mg/kg to 200 mg/kg, 200 mg/kg to 500 mg/kg, 200 mg/kg to 450 mg/kg, 200 mg/kg to 400 mg/kg, 200 mg/kg to 300 mg/kg, 300 mg/kg to 500 mg/kg, 300 mg/kg to 450 mg/kg, 300 mg/kg to 400 mg/kg, 400 mg/kg to 500 mg/kg, 400 mg/kg to 450 mg/kg, or 450 mg/kg to 500 mg/kg subject body weight, such as about 100, 125, 150, 200, 250, 300, 350, 400, 450, 500, or 600 mg/kg subject body weight. In particular examples, the poloxamer is formulated for administration at a dosage of about or at 25-450 mg/kg, 25-50 mg/kg, 200-450 mg/kg, such as 400 mg/kg subject body weight. Dosage will depend upon the rout of administration, and the goal is to achieve the target concentration of at least 0.05 mg/ml, particularly, 0.5 mg/ml to 1.5 mg/ml, for at least several hours, generally at least 12 hours, and up to 72 hours, including 1 day, 2 days, 3 days or 4 days to effect treatment.

In general, a goal is to administer the dose in the smallest volume possible. Typically, the volume to be administered is not greater than 3.0 mL/kg of a subject. For example, the volume in which the dose is administered to a subject can be 0.4 mL/kg to 3.0 mg/kg, 0.4 mL/kg to 2.5 mL/kg, 0.4 mL/kg to 2.0 mL/kg, 0.4 mL/kg to 1.8 mL/kg. 0.4 mL/kg to 1.4 mL/kg, 0.4 mL/kg to 1.0 mL/kg, 0.4 mL/kg to 0.6 mL/kg, 0.6 mL/kg to 3.0 mL/kg, 0.6 mL/kg to 2.5 mL/kg, 0.6 mL/kg to 2.0 mL/kg, 0.6 mL/kg to 1.8 mL/kg, 0.6 mL/kg to 1.4 mL/kg, 0.6 mL/kg to 1.0 mL/kg, 1 mL/kg to 3 mL/kg, 1 mL/kg to 2.5 mL/kg, 1 mL/kg to 2.0 mL/kg, 1 mL/kg to 1.8 mL/kg, 1 mL/kg to 1.4 mL/kg, 1.4 mL/kg to 3.0 mL/kg. 1.4 mL/kg to 2.5 mL/kg, 1.4 mL/kg to 2.0 mL/kg, 1.4 mL/kg to 1.8 mL/kg, 1.8 mL/kg to 3.0 mL/kg, 1.8 mL/kg to 2.5 mL/kg, 1.8 mL/kg to 2.0 mL/kg, 2.0 mL/kg to 3.0 mL/kg, 2.0 mL/kg to 2.5 mL/kg, or 2.5 mL/kg to 3.0 mL/kg. For example, a composition with a concentration of 22.5% (i.e., 225 mg/mL) that is administered at 100 mg/mL would be administered in a volume of about 0.4 mg/mL to achieve that dose. The particular volume chosen is one that results in a desired target concentration of poloxamer in the circulation of the subject after administration. Again, the particular volume and dosage is a function of the target circulating concentration, which for treating complications of hemo-concentration is described herein.

The formulations used in the methods provided herein can be administered by any appropriate route, for example, orally, nasally, pulmonary, intrapulmonary, parenterally, intravenously, intradermally, subcutaneously, intraarticularly, intracisternally, intraocularly, intraventricularly, intrathecally, intramuscularly, intraperitoneally, intratracheally or topically, as well as by any combination of any two or more thereof, in liquid, semi-liquid or solid form and are formulated in a manner suitable for each route of administration. Multiple administrations, such as repeat administrations described herein, can be effected via any route or combination of routes. The most suitable route for administration will vary depending upon the side effect to be treated and needs of the individual.

Typically, the administered dose is administered as an infusion. Generally, the infusion is an intravenous (IV) infusion. The poloxamer can be administered as a single continuous IV infusion, a plurality of continuous IV infusions, a single IV bolus administration, or a plurality of IV bolus administration. In some examples, the poloxamer is administered by other routes of administration, for example, subcutaneous or intraperitoneal injection, to achieve the desired concentration of poloxamer in circulation in the subject after administration.

In some examples, the poloxamer is administered as an IV infusion. The infusion, to provide the appropriate dosage, can be provided to the subject over a time period that is 1 hour to 24 hours, 1 hour to 12 hours, 1 hour to 6 hours, 1 hour to 3 hours, 1 hour to 2 hours, 2 hours to 24 hours, 2 hours to 12 hours, 2 hours to 6 hours, 2 hours to 3 hours, 3 hours to 24 hours, 3 hours to 12 hours, 3 hours to 6 hours, 6 hours to 24 hours, 6 hours to 12 hours, or 12 hours to 24 hours, such as generally over a time period that is up to or is about 1 hour, 2 hours, 3 hours, 4 hours, 5 hours, 6 hours, 7 hours, 8 hours, 9 hours, 10 hours, 12 hours, 15 hours, 18 hours, 20 hours, 22 hours, or more. It is within the level of a treating physician to determine the appropriate time and rate of infusion that can be tolerated by a subject.

The infusion of poloxamer, such as P188 (e.g., LCMF P188), can be provided as a single infusion that is not repeated for at least a week. For example, a single dosage can be sufficient to provide an therapeutically effective amount of poloxamer, e.g., a target concentration of poloxamer in circulation of the subject at a desired time point after administration. In the examples provided herein, the dosage can be repeated once every week, once every 2 weeks, once every three weeks or once every 4 weeks. For example, the dose can be repeated between 1 week to 4 weeks after the previous dose, such that the dose is repeated within 7 days, 8 days, 9 days, 10 days, 11 days, 13 days, 14 days, 15 days, 16 days, 17 days, 18 days, 19 days, 20 days, 21 days, 22 days, 23 days, 24 days, 25 days 26 days, 27 days, 28 days, 29 days or 30 days following completion of the prior dose. The dose that is administered in the repeated dosing can be the same or different than the prior dose. For example, it can be increased or decreased from the prior dose. It is within the level of the treating physician to determine the appropriate frequency of administration and level or amount of dosages in repeated dosings.

The length of time of the cycle of administration can be empirically determined, and is dependent on the side effect to be treated, the severity of the side effect, the particular patient, and other considerations within the level of skill of the treating physician. The length of time of treatment with P188, such as a LCMF P188, can be one week, two weeks, one months, several months, one year, several years or more. For example, a P188, such as an LCMF P188, can be administered no more than once weekly, such every 7 days, 8 days, 9 days, 10 days, 11 days, 12 days, 13 days, 14 days or more, over a period of a year or more. If the side effects persist in the absence of discontinued treatment, treatment can be continued for an additional length of time. Over the course of treatment, evidence of the side effects and/or treatment-related toxicity or side effects can be monitored.

In addition, the cycle of administration can be tailored to add periods of discontinued treatment in order to provide a rest period from exposure to the treatment. The length of time for the discontinuation of treatment can be for a predetermined time or can be empirically determined depending on how the patient is responding or depending on observed side effects. For example, the treatment can be discontinued for one week, two weeks, one month or several months.

Typically, treatment will start when the patient is admitted to the hospital, but it can be started any time during hospitalization to meet the subject's needs. More generally, the dosing will start during the first 72 hours of hospitalization.

The effective amount of a poloxamer, such as P188, and in particular an LCMF P188 as provided herein, can be delivered alone or in combination with other agents for treating a disease or condition. It is within the level of a skilled artisan to choose a further additional treatment to administer in conjunction with a therapeutic regime employing a poloxamer, such as a P188, such as LCMF P188. The decision 1 depends on the particular side effect/complication treated, the particular subject, the age of the subject, the severity of the side effect and other factors. In addition, dosages will vary among species.

In some embodiments herein, the poloxamer is administered to the subject in combination with another active agent, such as a diuretic, for treatment of an underlying condition. The poloxamer can be administered to the subject prior to, concomitant with, or after administration of the other agent, for example, a diuretic. For example, poloxamer, such as P188, such as LCMF P188, can be administered in combination with one or more diuretics, such as therapeutically effect amounts of a diuretic. For example, the methods in which the poloxamer P188, such as LCMF P188, is administered in combination with a diuretic are where diuretic therapy results in side effects, such as undesirable side effects, e.g., hemo-concentration and microvascular hemodynamic alterations. The poloxamer can be administered before or with the other agent to prevent the side effects/complications. Exemplary diuretics for use in the methods provided herein include, any known to those of skill in the art, and those described above. Exemplary of these, but not limited to, are, thiazide diuretics (e.g., bendroflumethiazide, hydrochlorothiazide, metolazone, and indapamide), loop diuretics (e.g., furosemide, bumetanide, and torasemide), potassium-sparing diuretics (e.g., spironolactone/eplerenone, amiloride, and triamterene), carbonic anhydrase inhibitors (e.g., acetazolamide, methazolamide, dorzolamide, topiramate, and elagitannins), osmotic diuretics (e.g., glucose and mannitol), and combinations thereof.

### E. METHODS OF ASSESSING HEMO-CONCENTRATION FOR TREATMENT

1. Assays of Hemo-Concentration

It is within the level of a skilled physician to assess hemo-concentration. Assays for hemo-concentration using clinical assessment include assays for pulse pressure and volume, skin turgor, dry mucous membranes, headache, hepatomegaly, central venous pressure, orthostatic hypotension, pruritus, splenomegaly, tachycardia, thirst, tinnitus, vertigo, and weakness. A number of such assays known to those of skill in the art are subject to quantitative analysis (e.g., pulse pressure and volume, venous pressure).

Assays for assessing hemo-concentration include those that assess hematocrit, erythrocyte volume fraction, erythrocyte sedimentation rate. For example, blood tests can be used to measure the level of red blood cells in the total blood volume.

In another method, the level of blood plasma proteins are measured using a blood test. In the assay, levels of blood plasma proteins, such as acute phase reactant proteins, e.g., fibrinogen, are measured and assessed to determine hemo-concentration. Increased levels of acute phase reactant proteins, such as fibrinogen, are indicative of hemo-concentration.

Other assays for hemo-concentration include those that assess hemodynamic performance, left ventricular-end diastolic volume, left ventricular-end systolic volume, and ejection fraction. For example, an echocardiography can be used to measure ejection fraction.

The ability of a poloxamer, such as a purified poloxamer 188, to exhibit therapeutic activity for treating or ameliorating hemo-concentration can be assessed using any one or more of the assays described above. In one example, a purified poloxamer 188 can be administered to a subject with hemo-concentration, or an appropriate animal model, and the effect on hemo-concentration can be assessed using transthoracic echocardiography and compared to subjects or animal models not administered a purified poloxamer 188.

The ability of a poloxamer 188, such as a purified poloxamer 188, to affect any one or more of the properties associated with hemo-concentration described above can be assessed using any one or more of the assays described above. The methods can be used to assess hemo-concentration in any subject with diuresis, including diuresis induced by diuretic treatment.

2. Assays of Dehydration

It is within the level of a skilled physician to assess dehydration. Assays for dehydration using clinical assessment include assays for mental status, vital signs, including heart rate, breathing rate, blood pressure, and temperature, thirst, dry mouth and swollen tongue, weakness, dizziness, heart palpitations, confusion, sluggishness, fainting, ability to sweat, diarrhea, fever, vomiting, weight loss, urine production, and seizures. A number of such assays known to those of skill in the art are subject to quantitative analysis (e.g., heart rate, blood pressure, urine production).

Assays for assessing dehydration include those that assess hemoglobin and red blood cell count. For example, blood tests can be used to measure the level of red blood cells in the total blood volume. Other assays include those that assess electrolyte levels, such as sodium, potassium and chloride levels and sugar levels. Assays for assessing dehydration include those that assess blood urea nitrogen (BUN) and creatine levels. For example, kidney function tests can be used to assess such levels. Other assays include urinalysis assays that assess the color, clarity, specific gravity, and presence of ketones in the urine. Assays for assessing dehydration are known to those of skill in the art.

Other assays for dehydration include those that assess hemodynamic performance, left ventricular-end diastolic volume, left ventricular-end systolic volume, and ejection fraction. For example, an echocardiography can be used to measure ejection fraction.

The ability of a poloxamer 188, such as a purified poloxamer 188, to exhibit therapeutic activity for treating or ameliorating dehydration can be assessed using any one or more of the assays described above. In one example, a purified poloxamer 188 can be administered to a subject with dehydration, or an appropriate animal model, and the effect on dehydration can be assessed using transthoracic echocardiography and compared to subjects or animal models not administered a purified poloxamer 188.

The ability of a poloxamer 188, such as a purified poloxamer 188, to affect any one or more of the properties associated with dehydration described above can be assessed using any one or more of the assays described above. The methods can be used to assess dehydration in any subject, including subjects with diuresis, such as diuresis induced by diuretic treatment.

### F. METHODS OF TREATING THE SIDE EFFECTS OF DIURESIS

Provided herein are methods of treatment and therapeutic uses for treating or ameliorating the side effects of diuresis. As discussed, diuresis can occur as a result of administration of certain drugs, for example, diuretics. Diuretics inhibit or reduce sodium (Na⁺) and water reabsorption by the nephrons of the kidney and are typically administered to subjects with clinical evidence of excess fluid, for example, subjects suffering from a medical condition such as kidney and liver related conditions, high blood pressure (i.e., hypertension), glaucoma, increased intra-ocular pressure, and heart-related conditions, for example, congestive heart failure. Though beneficial, diuresis can also result in unwanted side effects or consequences.

Generally, prior to treatment, patients are selected who exhibit one or more unwanted side effects associated with diuresis. It is within the level of a skilled physician to diagnose such unwanted side effects. For example, subjects that have an electrolyte imbalance, dehydration, arrhythmia, an elevated erythrocyte sedimentation rate, alterations of plasma volume, hemo-concentration of blood plasma proteins and/or blood cells, impaired microcirculation, or combinations thereof, are unwanted side effects associated with diuresis.

The methods and uses provided herein are for treating subjects that exhibit unwanted side effects associated with diuresis, e.g., diuretic-induced diuresis, including, but not limited to, electrolyte imbalance, dehydration, arrhythmia, alterations of plasma volume, hemo-concentration of blood plasma proteins and/or blood cells, microvascular hemodynamic dysfunction, and any other side effect or unwanted consequence associated with diuresis. In particular, the methods provided herein can be used in the treatment of subjects in which there is an increased level of blood cells, especially red blood cells, and plasma proteins in the blood, such as subjects with impaired circulation, particularly microcirculation. The methods provided herein can be used in the treatment of subjects in which there is an elevated erythrocyte sedimentation rate.

Diuresis typically results from diuretic treatment, although diuresis resulting from any other condition or circumstance is contemplated for use with the methods provided herein. In the methods and uses provided herein, poloxamer 188, such as a purified poloxamer 188, can be used to treat hemo-concentration of one or more blood plasma proteins, red blood cells, or a combination thereof, improve impaired circulation, particularly the microcirculation, treat dehydration, and any combination thereof. Thus, in methods provided herein, poloxamer 188, such as purified poloxamer 188, is used to treat unwanted side effects and consequences associated with diuresis, including diuretic-induced diuresis.

In particular, the methods include administration of a poloxamer such that the administration is sufficient to result in a concentration of the poloxamer in the circulation of the subject of from at or about 0.05 mg/mL to at or about 15 mg/mL, for example, from at or about 0.2 mg/mL to at or about 4.0 mg/mL, such as at or about 0.5 mg/mL. For example, the concentration of the poloxamer in the circulation of the subject can be representative of a single time point or representative of a mean steady state concentration that is maintained for a period of time, for example, up to 72 hours or more after administration.

In some of the methods provided herein, administration of the poloxamer is in combination with diuretic therapy or other therapy. In particular, the methods include administration of a therapeutically effective amount of a poloxamer, such as poloxamer 188 (P188), for example, any P188 described herein, and a therapeutically effective amount of a diuretic, for example, a thiazide diuretic, a loop diuretic, a potassium-sparing diuretic, a carbonic anhydrase inhibitor, an osmotic diuretic, and combinations thereof. The method includes administration of the poloxamer, e.g., P188, such that the administration is sufficient to result in a concentration of the poloxamer in the circulation of the subject of from at or about 0.05 mg/mL to at or about 15 mg/mL, for example, from at or about 0.2 mg/mL to at or about 4.0 mg/mL, such as at or about 0.5 mg/mL.

A concentration of 0.5 mg/ml can be maintained, for example, by administering an IV infusion of 50 mg/kg/hr. A plasma concentration of 1.0 mg/ml can be maintained by administering 100 mg/kg/hr. Other dosages can be readily extrapolated. In general the infusion could be continued between 12-48 hours as needed. Alternatively, repeat bolus administrations can be employed. For example, 50 mg/kg as an IV bolus can be administered every 6 hours for 1-3 days or 100 mg/kg every 6 hours for 1-3 days would result in concentrations in the middle of the desired range. Specific dosages and regiments readily can be determined. Treatment can be monitored by any method known to those of skill in the art, including those described herein For example, a positive treatment response can include either showing an improvement in StO2 (achieving or heading to a normal or baseline range) or normalizing the RBC sedimentation rate. Treatment can be continued until parameters approach or at normal levels or ranges.

1. Exemplary Side Effects and Complications that can be Treated

a. Hemo-Concentration

Provided herein is a method of reducing, lessening, ameliorating or treating hemo-concentration, such s that caused by diuresis by administering a poloxamer, such as a purified poloxamer 188, to a subject. Hemo-concentration is a relative increase in the concentration of cellular elements in the blood, in particular, erythrocytes (i.e., red blood cells) and blood plasma proteins, such as positive acute phase reactant proteins. Generally, hemo-concentration is attributable to the loss of blood plasma. For example, administering diuretics to a subject can lead to increased urine production (i.e., diuresis) and rapid water loss, thus leading to an increase in the concentration of red blood cells and plasma proteins, causing hemo-concentration. In particular, provided herein are methods of using poloxamer 188, such as a purified poloxamer 188, for treating, ameliorating or reducing hemo-concentration. Subjects or patients with hemo-concentration can be administered a poloxamer 188, such as a purified poloxamer 188.

The methods and uses provided herein are for treating subjects that typically exhibit symptom(s) associated with hemo-concentration. In particular, the methods herein can be used to treat patients with hemo-concentration of blood plasma proteins, for example, positive acute phase reactant proteins, including, but not limited to, C-reactive protein (CRP), serum amyloid P, serum amyloid A, complement factors, fibrinogen, prothrombin, anti-hemophilic factor (AHF), von Willebrand factor, mannan-binding lectin, plasminogen, alpha 2-macroglobulin, ferritin, hepcidin, ceruloplasmin, haptoglobin, alpha-1-acid glycoprotein (AGP), alpha 1-antitrypsin, alpha 1-antichymotrypsin, and plasminogen activator inhibitor I, and/or blood cells, for example, erythrocytes. Generally, prior to treatment, patients are selected that exhibit one or more signs or symptoms associated with hemo-concentration. It is within the level of a skilled physician to diagnose hemo-concentration. Subjects that have hemo-concentration, including hemo-concentration of blood plasma proteins and red blood cells, generally exhibit increased levels of one or more acute phase reactant proteins, e.g., fibrinogen, or blood cells, e.g., erythrocytes (i.e., red blood cells), or any combination thereof. For example, hemo-concentration can be reflected as an elevated hematocrit or erythrocyte sedimentation rate, where the rate can be used as an indirect measurement of the presence of pro-sedimentation factors in the blood, e.g., fibrinogen. Selection of a subject having hemo-concentration for treatment with a poloxamer 188, such as a purified poloxamer 188, in the methods provided herein can be based on clinical symptoms, hematocrit measurements, or levels of acute phase reactant proteins, for example, in the blood.

In particular, provided herein are methods of using a poloxamer 188, such as a purified poloxamer 188, for treating, ameliorating or reducing hemo-concentration induced by diuresis. Subjects or patients with hemo-concentration can be administered a poloxamer 188, such as a purified poloxamer 188.

b. Dehydration

Provided herein is a method of reducing, lessening, ameliorating or treating dehydration by administering a poloxamer 188, such as a purified poloxamer 188, to a subject. Dehydration is the excessive loss of body water coupled with a disruption of the metabolic process. Dehydration can refer to any condition where fluid volume is reduced. Most commonly, dehydration refers to hypernatremia, where there is a loss of water and accompanying excess concentration of salt, but can also refer to hypovolemia, where there is a loss of blood volume, particularly, plasma. In some examples, dehydration is caused by diuresis, e.g., diuretic-induced diuresis. For example, administering diuretics to a subject cause the kidneys to excrete more sodium into the urine, which then leads to increased urine production (i.e., diuresis) and rapid water loss, thus leading to dehydration. In particular, provided herein are methods of using poloxamer 188, such as a purified poloxamer 188, for treating, ameliorating or reducing dehydration. Subjects or patients with dehydration can be administered a poloxamer 188, such as a purified poloxamer 188.

The methods and uses provided herein are for treating subjects that typically exhibit symptom(s) associated with dehydration. In particular, the methods herein can be used to treat patients with dehydration that occurs as the result of diuretic therapy although dehydration resulting from any other condition or circumstance is contemplated for use with the methods provided herein. Generally, prior to treatment, patients are selected that exhibit one or more signs or symptoms associated with dehydration. It is within the level of a skilled physician to diagnose dehydration. Subjects that are dehydrated generally exhibit mild to severe symptoms, including, but not limited to, increased thirst, dry mouth and swollen tongue, weakness, dizziness, heart palpitations, confusion, sluggishness, fainting, inability to sweat, severe diarrhea, fever, increased or constant vomiting, weight loss, decreased urine production, seizures, or any combination thereof. For example, dehydration can be reflected by moderate to severe diarrhea, e.g., diarrhea that lasts for at least two days, and fever. Selection of a subject having dehydration for treatment with a poloxamer 188, such as a purified poloxamer 188, in the methods provided herein can be based on clinical symptoms, such as mental status, vital signs, including heart rate, breathing rate, blood pressure, and temperature, blood tests to determine hemoglobin and red blood cell count, urinalysis and kidney function tests.

In particular, provided herein are methods of using a poloxamer 188, such as a purified poloxamer 188, for treating, ameliorating or reducing dehydration, such as dehydration induced by diuresis. Subjects or patients with dehydration can be administered a poloxamer 188, such as a purified poloxamer 188.

2. Identification of Subjects for Treatment

a. Identifying Subjects with Hemo-Concentration

Identification of a subject with hemo-concentration for treatment with a poloxamer, such as a purified poloxamer 188, can be based on symptoms such as decreased pulse pressure and volume, loss of skin turgor, dry mucous membranes, headaches, hepatomegaly, low central venous pressure, orthostatic hypotension, pruritus (especially after a hot bath), splenomegaly, tachycardia, thirst, tinnitus, vertigo, and weakness.

Selection of a subject having hemo-concentration for treatment with poloxamer, such as a purified poloxamer 188, in the methods provided herein can be based on an increased hematocrit (percentage of red blood cells in whole blood) or erythrocyte volume fraction, which can be determined by a blood test. Such techniques are well known to one of skill in the art. Normal hematocrit levels are typically between 40.7% and 50.3% for adult males, 36.1% to 44.3% for adult females, 45% to 61% for newborns, and 32% to 42% for infants. Indicative of hemo-concentration includes an increased erythrocyte (i.e., red blood cell) sedimentation rate, where the rate can be used as an indirect measurement of the presence of pro-sedimentation factors in the blood, e.g., fibrinogen.

Identification can be based on increased levels of blood plasma proteins, including acute phase reactant proteins, such as C-reactive protein (CRP), serum amyloid P, serum amyloid A, complement factors, fibrinogen, prothrombin, anti-hemophilic factor (AHF), von Willebrand factor, mannan-binding lectin, plasminogen, alpha 2-macroglobulin, ferritin, hepcidin, ceruloplasmin, haptoglobin, alpha-1-acid glycoprotein (AGP), alpha 1-antitrypsin, alpha 1-antichymotrypsin, and plasminogen activator inhibitor I. Increased levels of acute phase reactant proteins, such as fibrinogen, are indicative of hemo-concentration. Blood plasma protein levels can be determined by a blood test and any other method known to those of skill in the art.

In exemplary methods to identify a subject with hemo-concentration of erythrocytes and/or blood plasma proteins for treatment, blood tests and other assays and methods are generally performed prior to, during, or following treatment of the subject with a poloxamer, such as a purified poloxamer 188. In exemplary methods of monitoring therapy for hemo-concentration, blood samples or other assays can be performed before, during or after the subject has received one or more treatments with the poloxamer.

Identification of a subject with hemo-concentration for treatment with a poloxamer, such as a purified poloxamer 188, in the methods provided herein can be based on indicators of impaired or dysfunctional microcirculation or microvascular hemodynamics, including blood pressure, heart rate, perfused capillary density, and any other indicator of impaired or dysfunctional microcirculation or microvascular hemodynamics known to those of skill in the art.

b. Identifying Subjects with Dehydration

Identification of a subject with dehydration for treatment with poloxamer, such as a purified poloxamer 188, in the methods provided herein can be based on clinical symptoms such as mental status, vital signs, including heart rate, breathing rate, blood pressure, and temperature, increased thirst, dry mouth and swollen tongue, weakness, dizziness, heart palpitations, confusion, sluggishness, fainting, inability to sweat, severe diarrhea, fever, increased or constant vomiting, weight loss, decreased urine production, seizures, or any combination thereof.

Identification also can be based on results obtained from blood tests, e.g., increased hemoglobin and red blood cell count, electrolyte levels (e.g., sodium, potassium, and chloride), sugar levels, and complete blood count; urinalysis tests, e.g., color, clarity, specific gravity, presence of ketones; and kidney function tests, e.g., BUN and creatine levels. Dehydration can be determined by any method known to those of skill in the art.

In exemplary methods to select a subject with dehydration for treatment, blood tests and other assays and methods are generally performed prior to, during, or following treatment of the subject with a poloxamer, such as a purified poloxamer 188. In exemplary methods of monitoring therapy for dehydration, blood samples or other assays can be performed before, during or after the subject has received one or more treatments with a poloxamer 188, such as a purified poloxamer 188.

Hence for treatment with and uses of poloxamers as described herein, clinical indicators, include, but are not limited to: clinical tests, such as sedimentation rates, a decline in or a low value for StO₂ (tissue oxygenation), laboratory measurements showing elevated fibrinogen, elevated RBC count, elevated hematocrit (any value above normal), laboratory measurement of RBC aggregation (showing increased aggregation) or RBC sed rate (elevated, anything above the normal range)

3. Monitoring Subjects for Treatment

A poloxamer 188, such as a purified poloxamer 188 provided herein, reduces, lessens or ameliorates unwanted side effects associated with diuresis, including, but not limited to, dehydration, hemo-concentration of blood plasma proteins and/or blood cells, and impaired microvascular hemodynamic function. A subject can be monitored over time to assess whether a decrease in the unwanted side effects has been achieved over the course of therapy with a poloxamer 188, such as a purified poloxamer 188, provided herein.

### G. COMBINATION TREATMENTS

Poloxamer 188, such as any poloxamer 188 described herein, can be administered in combination with therapeutics previously utilized to treat kidney and liver related conditions, high blood pressure (i.e., hypertension), glaucoma, increased intra-ocular pressure, and heart-related conditions, such as congestive heart failure, in order to improve the efficacy of the poloxamer 188 compound on its own. Typically, such treatments include, but are not limited to, methods of treatment of physiological and medical conditions described and listed herewith. The compositions provided herein can be further co-formulated or co-administered together with, prior to, intermittently with, or subsequent to, other therapeutic or pharmacologic agents or treatments, such as treatments where reduced hemo-concentration is desired. For example, poloxamer 188, such as any poloxamer 188 described herein, can be used in the treatment of the side effects of diuresis, for example hemo-concentration, dehydration, and/or microvascular hemodynamic dysfunction.

A preparation of a second agent or agents or treatment or treatments can be administered at once, or can be divided into a number of smaller doses to be administered at intervals of time. Selected agent/treatment preparations can be administered in one or more doses over the course of a treatment time, for example, over several hours, days, weeks, or months. In some cases, continuous administration is useful. It is understood that the precise dosage and course of administration depends on the indication and patient's tolerability. Generally, dosing regimens for second agents/treatments herein are known to one of skill in the art.

Poloxamer 188, such as a purified poloxamer 188 described herein, also can be used in conjunction with currently available therapeutics, including, but not limited to: diuretics, such as the diuretics described herein, vasodilators, ACE inhibitors, ARBs (angiotensin receptor blockers), angiotensin H antagonists, aldosterone antagonists, positive inotropic agents, phosphodiesterase inhibitors, beta-adrenergic receptor antagonists, calcium channel blockers, nitrates, alpha blockers, central alpha antagonists, statins, digoxin, nitrates, chlorthalidone, amlodipine, lisinopril, doxazocin, or a combination of these agents. Additionally, poloxamer 188, such as a purified poloxamer 188 described herein, also can be used in conjunction with mechanical devices, including: implantable pacemakers, defibrillators, and left ventricular assist devices (LVAD).

### H. EXAMPLES

The following examples are included for illustrative purposes only and are not intended to limit the scope of the invention.

### Example 1

### Purification of a Longer Circulating Material Free (LCMF) Poloxamer 188 Using a 50-L Scale Multi-Step Extraction Batch Process Purification with Higher Methanol Concentrations and Lower Pressure

A. Supercritical Fluid Extraction (SFE) Process

A multi-step extraction batch process of poloxamer 188 was performed with extraction conducted at a pressure of 247±15 atm (approximately 250 bar) and a controlled step-wise increase of methanol of 7.4, 9.1 and 10.7 weight % methanol. Before purification, the poloxamer 188 raw material (BASF Corporation, Washington, N.J.) was characterized by Gel Permeation Chromatography (GPC). Molecular weight analysis demonstrated that raw material had an average molecular weight of the main peak of about 8,500±750 Da, no more than 6.0% low molecular weight (LMW) species of less than 4,500 Da and no more than 1% high molecular weight species (HMW) greater than 13,000 Da. In addition, the polydispersity was no more than 1.2.

A 50-L, high pressure, stainless steel, extractor vessel was charged with 14 kg of commercial grade poloxamer 188 (BASF Corporation, Washington, N.J.) and 7 kg of methanol, pressurized with CO₂ (49±10 atm, i.e. 720±147 psi) (Messer France, S.A.S., Lavera, France) and heated to 40° C. for 40-80 minutes until a homogenous solution was obtained. CO₂ (supplied either from a main supply tank or via recycling through an extraction system), was cooled in a heat exchanger and fed into a temperature-controlled, high pressure, stainless steel, solvent reservoir. A high-pressure pump increased the pressure of liquid CO₂ to the desired extraction pressure. The high pressure CO₂ stream was heated to the process temperature by a second heat exchanger. Methanol (Merck KGaA, Darmstadt, Germany) was fed from a main supply tank into the CO₂ solvent stream to produce the extraction methanol/CO₂ cosolvent, which was fed through inlet systems into the extractor vessel as a fine mist at a pressure of 247±15 atm (3600±psi) and a temperature of 40° C.

A 7.4% methanol/CO₂ extraction cosolvent was percolated through the poloxamer solution for 3 hours at a methanol flow rate of 8 kg/hr (108 kg/hr total flow rate). The extraction continued with a 9.1% methanol/CO₂ cosolvent for 4 more hours at a methanol flow rate of 10 kg/hour (110 kg/hr total flow rate). The extraction further continued with a 10.7% methanol/CO₂ cosolvent for 8 more hours at a methanol flow rate of 12 kg per hour (112 kg/hr total flow rate). Throughout the extraction process, extraction of soluble species were continuously extracted from the top of the extractor. The extraction solvent was removed from the top of the extractor and passed through two high pressure, stainless steel, cyclone separators arranged in series to reduce system pressure from 247 atm (3600 psi) to 59 atm (870 psi) and then from 59 atm to 49 atm (720 psi) and to separate CO₂ from the methanolic stream. The separated CO₂ was condensed, passed through the heat exchanger and stored in the solvent reservoir. Pressure of the methanol waste stream was further reduced by passing through another cyclone separator. The purified poloxamer 188 remained in the extractor.

After extraction, the purified poloxamer 188 solution was discharged from the bottom of the extractor into a mixer/dryer unit equipped with a stirrer. The poloxamer 188 product was precipitated under reduced pressure via a Particle from Gas Saturated Solutions (PGSS) technique. The precipitate contained approximately 25% to 35% methanol. The purified poloxamer 188 was dried under vacuum at not more than 40° C. to remove residual methanol. The feed yield of the product was about 65%.

Molecular weight analysis of the purified product as determined by GPC demonstrated that the purified product met the acceptance specifications. There was an average molecular weight of the main peak of about 8,500±750 Da and an average molecular weight average of 8,500±750 Da, no more than 1.5% low molecular weight (LMW) species of less than 4,500 Da and no more than 1.5% high molecular weight species (HMW) greater than 13,000 Da. In addition, the polydispersity was no more than 1.05. Thus, the results showed that the procedures resulted in a measurable reduction in the LMW species, and an improvement in the polydispersity of the purified product. The resulting Poloxamer 188 was a clear, colorless, sterile, non-pyrogenic, aqueous solution in 100 mL glass vials containing 15 g of purified poloxamer drug, substance (150 mg/mL). The composition contained 0.01 M citrate buffer and sodium chloride to adjust the total sodium content to be equivalent to that in 0.45% sodium chloride solution for injection. The resulting osmolarity of the solution was approximately 312 mOsm/L. The LCMF poloxamer-188 composition did not contain any bacteriostatic agents or preservatives.

B. Characterization of Plasma Circulating Half-Life Following Intravenous Administration of Purified Poloxamer 188

Purified poloxamer 188 is reported to result in two distinct peaks in the circulation that exhibit different pharmacokinetic profiles, a main peak with an average peak molecular weight of 8,600 daltons and a smaller high molecular weight (HMW) peak with an average molecular weight of about 16,000 daltons (Grindel et al. (2002) Biopharmaceutics and Drug Disposition, 23:87-103). The higher molecular weight peak exhibits a longer plasma residence time with slower clearance from the circulation such that it is cleared at approximately 5% of the main peak plasma clearance rate. The circulating half-life of purified poloxamer 188 was assessed following intravenous administration.

Purified poloxamer 188 generated as described above was administered intravenously to healthy human volunteers. The purified poloxamer 188 was administered as a loading dose of 100 mg/kg/hr for one hour followed by a maintenance dose of 30 mg/kg/hr for 5 hours. Plasma was collected at various time points and the plasma concentration of poloxamer 188 was determined using HPLC-GPC. The results are set forth in FIG. 7. Consistent with reported studies regarding the half-life of the main peak, the results demonstrate a mean maximum concentration (Cmax) of the administered purified poloxamer 188 of 0.9 mg/mL was attained by the end of the one hour loading infusion. Also, a mean concentration at steady state (Css) of about 0.4 mg/mL was attained during maintenance infusion with the concentration declining rapidly following discontinuation of the maintenance infusion. Unlike previous reports, the product purified as described above did not result in any observed longer circulating higher molecular weight peaks in the plasma.

To confirm the absence of longer circulating molecular weight peaks in plasma, purified poloxamer 188 prepared as described above was administered as a loading dose of 300 mg/kg/hr for one hour followed by a maintenance dose of 200 mg/kg/hr for 5 hours. Plasma was collected at various time points and the plasma concentrations using HPLC-GPC. The results are set forth in FIGS. 8A and 8B, which depicts HPLC-GPC chromatograms of plasma obtained at various time points following administration of the purified poloxamer 188 prepared as described in above. FIG. 8A depicts the plasma concentration time course for the entire molecular weight distribution as measured by HPLC-GPC for all plasma sampling time points. FIG. 8B depicts selected time points illustrating the change in the chromatographic profile over time. The chromatograms are enlarged to show the high molecular weight portion (19.8 K daltons-12.4 K daltons) of the polymeric distribution. Also shown are the main peak portion (molecular weight range of approximately 12.8 K daltons to approximately 4.7 K daltons) and the lower molecular weight portion (those components with molecular weights <approximately 4.7 K daltons.). The HPLC-GPC method quantifies plasma levels based on the height of the eluting peak relative to standards of known concentration (i.e. the higher the eluting peak, the higher the plasma level). The GPC method also identifies the molecular weight range by comparison of the sample elution time to that of standards of known molecular weight.

The chromatograms show that over time the high molecular weight portion of the poloxamer 188 polymeric distribution declines in relative proportion to the main peak and lower molecular weight components. Thus, the polymeric distribution shows a substantially uniform pharmacokinetic profile. Thus, the results show that the higher molecular weight species do not exhibit a longer circulating half-life (relative to the other polymeric components) and do not accumulate in the circulation following intravenous administration. Thus, the poloxamer 188 is designated longer circulating material free (LCMF) poloxamer 188.

### Example 2

### Diuretic Induced Hemo-Concentration

A 65-year-old male weighing 180 pounds, with a history of a prior myocardial infarction, was hospitalized with fatigue, peripheral edema and difficulty in breathing. A chest x-ray revealed interstitial edema and pulmonary venous congestion and a transthoracic echocardiography showed poor left ventricular contractility with an ejection fraction of 30%. Diuretic therapy effected by administering IV bolus doses of 150 mg furosemide every 12 hours was started. His body weight measurement after 48 hours of furosemide treatment was 172 pounds. At this time, a repeat transthoracic echocardiography showed essentially no change from the prior evaluation (the patient's ejection fraction was 29%).

The patient was continued on furosemide and a 15% solution of purified poloxamer 188 in citrate buffered saline was administered at a dose of 250 mg/kg as an IV infusion over a period of 1 hour. Twenty-four hours later, his body weight was 168 pounds. Transthoracic echocardiography at this time showed a marked reduction in the dilated myopathy and significant improvement in ventricular contractility, with an improved ejection fraction of 40%. His clinical symptoms were markedly improved and he was discharged from the hospital. One week later, the patient received a follow-up transthoracic echocardiography which showed an ejection fraction of 37%.

### Example 3

### Dehydration in a Patient

A 72-year-old female presented to an emergency room with a complaint of weakness and fatigue. She was observed to have shortness of breath while walking. The patient had no history of congestive heart failure and was taking an ACE inhibitor. For the preceding 2-3 days, she had experienced moderate to moderately severe diarrhea and a low grade fever. She had trace edema of the lower extremity. A bedside echocardiogram showed an ejection fraction of 28%. Laboratory studies showed a high hematocrit and an elevated erythrocyte sedimentation rate.

The patient was treated with purified poloxamer 188 administered as a 10% solution in citrate buffered saline. The dose of 50 mg/kg was delivered as an IV infusion over a period of 1 hour using an infusion pump. Two hours post-infusion, a repeat echocardiogram showed her ejection fraction had improved to 38%. Laboratory studies on blood taken at the 2-hour post-infusion time point revealed no change n hematocrit but a normal erythrocyte sedimentation rate.

Since modifications will be apparent to those of skill in this art, it is intended that this invention be limited only by the scope of the appended claims.

1. A method for treating or preventing hemo-concentration comprising:
administering to a subject experiencing hemo-concentration or undergoing a treatment that causes hemo-concentration a therapeutically acceptable amount of a polyoxyethylene/polyoxypropylene copolymer having the formula:
HO(C₂H₄O)_{a′}—(C₃H₆O)_{b}—(C₂H₄O)ₐH;
a′ and a are the same or different and each is an integer such that the hydrophile portion represented by (C₂H₄O) constitutes 60% to 90% or about 60% to about 90% by weight of the compound;
b is an integer such that the hydrophobe represented by (C₃H₆O) has a molecular weight of approximately or 1300 to 2300 Daltons (Da); and
the polydispersity value is less than or equal to 1.07.

2. The method of claim 1, wherein the molecular weight of the hydrophobe (C₃H₆O) is approximately or is 1750 Da and the total molecular weight of the copolymer is approximately or is 8400 to 8800 Da.

3.-6. (canceled)

7. The method of claim 1, wherein the polyoxyethylene/polyoxypropylene copolymer is a poloxamer with a hydrophobe having a molecular weight of about 1400 to 2000 Da or 1400 to 2000 Da, and a hydrophile portion constituting approximately 70% to 90% or 70% to 90% by weight of the copolymer.

8. The method of any claim 7, wherein the copolymer comprises poloxamer 188.

9. The method of claim 7, wherein
the hydrophobe represented by (C₃H₆O) has a molecular weight of approximately 1700 to 1800 Da, and the poloxamer has a total molecular weight between 8400 to 8800 Da; or
the hydrophile constitutes 80% to 81% of the total molecular weight of the poloxamer, and the molecular weight of the hydrophobe is 1750 Da, and the poloxamer has a total molecular weight between 8400 to 8800 Da; or
the hydrophile constitutes 80% to 81% of the total molecular weight of the poloxamer; and the molecular weight of the hydrophobe is 1800 Da, and the poloxamer has a total molecular weight between 8400 to 8800 Da.

10.-18. (canceled)

19. The method of claim 9, wherein:
the percentage of high molecular weight components in the preparation greater than 13,000 Da constitutes less than 1% of the total distribution of components of the poloxamer preparation.

20.-21. (canceled)

22. The method of claim 1, wherein the polyoxyethylene/polyoxypropylene copolymer is administered in a composition at concentrations from 10.0 mg/mL to 200.0 mg/mL.

23. The method of claim 1, wherein the hemo-concentration results from diuresis and/or dehydration.

24. The method of claim 1, wherein the hemo-concentration results from diuresis.

25.-29. (canceled)

30. The method of claim 24, wherein said subject has or is receiving diuretic treatment administered to ameliorate a condition selected from among a kidney related condition, high blood pressure, a liver condition, a heart-related condition and glaucoma.

31.-34. (canceled)

35. The method of claim 30, wherein the subject has a disease or condition selected from atherosclerosis, diabetes, heart failure, vasculitis, Raynaud's disease, sickle cell disease and polycythemia.

36.-49. (canceled)

50. The method of claim 1, wherein the copolymer is administered by intravenous infusion.

51.-56. (canceled)

57. The method of claim 1, wherein the subject treated is a human.

58.-114. (canceled)

115. The method of claim 9, wherein the polyoxyethylene/polyoxypropylene copolymer is administered in a composition at concentrations from 10.0 mg/mL to 200.0 mg/mL.

116. The method of claim 9, wherein the hemo-concentration results from diuresis and/or dehydration.

117. The method of claim 9, wherein the hemo-concentration results from diuresis.

118. The method of claim 9, wherein said subject has or is receiving diuretic treatment administered to ameliorate a condition selected from among a kidney related condition, high blood pressure, a liver condition, a heart-related condition and glaucoma.

119. The method of claim 9, wherein the subject has a disease or condition selected from atherosclerosis, diabetes, heart failure, vasculitis, Raynaud's disease, sickle cell disease and polycythemia.

120. The method of claim 9, wherein the copolymer is administered by intravenous infusion.

121. The method of claim 9, wherein the subject treated is a human.

122.-135. (canceled)
